(19) United States
(12) Patent Application Publication
(10) Pub. No.: US 2025/0297152 A1
(43) Pub. Date: Sep. 25, 2025

(54) FLUORINATED SUBSTITUTED OLEFIN REFRIGERANTS AND METHODS OF COOLING ELECTRONICS

(71) Applicant: Honeywell International Inc., Charlotte, NC (US)

(72) Inventors: Iamnica J. Linares Mendez, Buffalo, NY (US); Christophe Roger, Buffalo, NY (US); Dimitrios Papanastasiou, Buffalo, NY (US); Sudharsanam Ramanathan, Buffalo, NY (US); Vinayak Krishnamurti, East Amherst, NY (US)

(21) Appl. No.: 19/062,284

(22) Filed: Feb. 25, 2025

Related U.S. Application Data
(60) Provisional application No. 63/558,791, filed on Feb. 28, 2024.

(51) Int. Cl. C09K 5/04

(52) U.S. Cl. CPC C09K 5/045; C09K 2205/126; C09K 2205/22

(57) ABSTRACT

The compounds trans and cis-1,3,4,4,5,5,5-heptafluoro-3-(trifluoromethyl)pent-1-ene and trans and cis-1,4,4,4-tetrafluoro-3,3-bis(trifluoromethyl)but-1-ene. Methods of heating and/or cooling of electronic components, articles and/or devices during the manufacture and/or operation thereof include providing an electronic component, article or device which is being manufactured and/or being operated for its intended purpose; and transferring heat to and/or from said electronic component, article and/or device during at least a portion of said manufacturing and/or operating process by directly or indirectly transferring heat between said electronic component, article and/or device and a refrigerant fluid comprising at least trans-1,3,4,4,5,5,5-heptafluoro-3-(trifluoromethyl)pent-1-ene or trans-1,4,4,4-tetrafluoro-3,3-bis(trifluoromethyl)but-1-ene.

### CROSS-REFERENCE TO RELATED APPLICATIONS

This application claims priority to U.S. Provisional Patent Application No. 63/558,791 entitled “FLUORINATED SUBSTITUTED OLEFIN REFRIGERANTS AND METHODS OF COOLING ELECTRONICS”, filed on Feb. 28, 2024, the entire disclosure of which is incorporated by reference in its entirety.

### FIELD

The present invention is related to new fluorine substituted olefins and to methods of using same in various applications, including as refrigerants, and especially in connection with the cooling and/or heating of electronics during manufacture thereof and/or during operation thereof.

### BACKGROUND

The industry continues to experience a need for heat transfer fluids which have low global warming potential while providing one or more of (and preferably all of) the following properties: high thermal stability, acceptable toxicity, nonflammability and effective heat transfer properties to meet the requirements of various applications. One particular application which presents an especially difficult challenge in this regard is the cooling (and in some cases also heating) of electronic components, devices and articles during the manufacture and/or during operation thereof. For example, the following electronic devices present a challenge to cool in manufacture and/or in operation: high-capacity energy storage devices, power electronics (TVs, cell phones, monitors, drones), battery thermal management (automotive and stationary), e-powertrain, IGBT, computer server systems, computer chips, 5G network devices, central processing units (CPUs) and displays. The challenge associated with cooling such equipment has increased, at least in part, because such electronic devices have been moving in a direction of higher and higher levels of performance in smaller and smaller packages (such as, for example, in high performance data center computing). This product progression has created the need for higher levels of heat transfer performance (while maintaining many of, and preferably all of, the other factors mentioned above) to ensure that such systems operate within the design temperature range.

By way of example, there has recently been interest in the possibility of providing much needed computing power by running CPUs in an overclocked condition by increasing core frequency and core voltage. However, while overclocking can provide the desired improvement in processing capacity, it also generally results in a concomitant need for improvements in cooling the component during operation to maintain the electronic component within temperature limits and to avoid unacceptable decreases in the reliability and longevity of the electronic component. In this regard, applicants have come to appreciate that advantages can be achieved if cooling fluids used for immersion cooling have boiling points less than 60° C. in order to achieve the coolest operating temperature and the most desirable levels of longevity and reliability. In addition, applicants have come to appreciate that while lower refrigerant boiling point temperatures can have beneficial effects, as boiling point temperatures begin to approach the temperature of the heat sink used to condense the refrigerant (e.g., cooling water), the heat transfer driving force (i.e., delta T) becomes a limiting factor. A significant challenge is thus presented to identify a new, low-GWP cooling fluid that has a boiling point less than about 60° C. while at the same time a sufficiently low dielectric constant to permit immersion cooling, as well as the other properties that are important for immersion cooling applications. For example, the cooling fluid sold by 3M as FC-3284 has a relatively acceptable dielectric constant of about 1.9 and boiling point of 50° C., but a high GWP. On the other hand, the material sold by 3M as HFE7000 has a relatively low boiling point of 34° C. and an undesirably high dielectric constant of 7.4.

Applicants have thus come to appreciate the need for refrigerants, methods and systems which are at once environmentally acceptable (low GWP and low ODP), are non-flammable, have acceptable toxicity, and have one or more properties needed for the particular application (for example, appropriate heat transfer properties for particular heat transfer applications (including sub-40° C. boiling points for high demand applications such as overclocking) and/or low dielectric constant if the application involves exposure or potential exposure to electronic equipment or components during operation (e.g., immersion cooling of electronic components).

A need also continues to exist for improved fluids to heat and/or cool (i.e., manage the temperature of) electronic components, devices, articles, and in the manufacturing process for such components, devices and articles. This is a substantial technical challenge since the refrigerant will frequently need to operate effectively over a relatively wide range of processing conditions, including process temperatures, and during potential exposure to electronics during said processing.

Examples of electronic manufacturing processes that experience thermal management challenges include the etching, rapid thermal annealing (RTA) and the like of semiconductor integrated circuitry, especially as the line width of such circuitry continues to decrease. These manufacturing challenges include an increasing need to achieve effective and relatively precise temperature control of certain of the fluids and/or components used in the manufacturing process. See for example U.S. Pat. No. 5,904,572 (relating to wet etching processes), U.S. 2005/0155555 (relating to vapor deposition in semiconductor manufacture) and U.S. 2007/0117362 (relating to RTA), each of which is incorporated herein by reference. These challenges are intensified because it is also required in certain electronics cooling applications that the viscosity of the refrigerant fluid being used to manage the temperature of the electronic component has a sufficiently low viscosity in the operating temperature range of the refrigerant fluid to allow the fluid to be circulated and to maintain its desired heat transfer properties.

Vapor phase soldering is another example of an electronics manufacturing process that utilizes refrigerants to help manage processing temperatures. In this application, high temperatures are used and accordingly the heat transfer fluid must be suitable for high temperature exposure (e.g., up to 250° C.). Currently, perfluoropolyethers (PFPE), compounds that have only carbon, oxygen and fluorine) are commonly used as the heat transfer fluids in this application. Although many PFPEs have adequate thermal stability for these high temperatures, they are environmentally persistent with extremely long atmospheric lifetimes which, in turn, gives rise to very high global warming potentials (GWPs).

Another example of the challenge in providing thermal management fluids is the increasing use of electronic vehicles, including particularly, cars, trucks, motorcycles and the like. In electric vehicles the thermal management function is especially important and challenging for several reasons, including the criticality of cooling and/or heating the batteries to be within a relatively narrow temperature range and in a way that is reliable, efficient and safe, and the challenge to provide effective thermal battery management is becoming greater as the demand for battery-operated vehicles with greater range and faster charging increases.

The efficiency and effectiveness of batteries, especially the batteries that provide the power in electronic vehicles, is a function of the operating temperature at which they operate. Thus, thermal management system must frequently be able to do more than simply remove heat from the battery during operation and/or charging—it must be able to effect cooling in a relatively narrow temperature range using equipment that is as low cost as possible and as light weight as possible. This results in the need for a heat transfer fluid in such systems that possesses a difficult-to-achieve combination of physical and performance properties. Furthermore, in some important applications the thermal management system must be able to add heat to the battery, especially as the vehicle is started in cold weather, which adds further to the difficulty of discovering and developing/obtaining compounds and/or compositions effective in such systems, not only from a thermal performance standpoint, but also a myriad of other standpoints, including environmental, safety, dielectric properties, and others.

As a particular example of the importance of dielectric constant, one frequently used system for the thermal management of electric vehicle batteries involves immersing the battery in the fluid used for thermal management. Such systems add the additional constraint that the fluid used in such systems must be electronically compatible with the intimate contact with the battery, or other electronic device or component, while the battery or device is in operation. In general, this means the fluid must not only be non-flammable, but must have a low electrical conductivity and a high level of stability while in contact with the battery or other electronic component(s) while the component(s) are operating and at the relatively high temperatures existing during operation. Applicants have come to appreciate the desirability of such properties even in indirect cooling of operating electronic devices and batteries because leakage of any such fluid may result in contact with operating electronic components.

Certain fluorinated compounds, including perfluorinated compounds, have heretofore frequently been used in many of the demanding applications mentioned above. It has been noted, however, that while many of such perfluorinated fluids (such as Fluorinert™ FC-72 and FC-3284) exhibit desirable dielectric properties (e.g., dielectric constants of 2.0 or less), these fluids are undesirable from the environmental standpoint since they are generally associated with very high GWP values. See, for example, US Patent Application 2023/0112841, which proposes the use of certain five (5) and six (6) carbon fluorinated olefins for use in an application involving immersion cooling. WO 2010/055146 also discloses numerous fluorinated olefins as refrigerants for use in cascade refrigeration systems; however, this publication neither recognizes the challenges disclosed herein with heat transfer in electronics, nor does it disclose immersion cooling techniques. U.S. Pat. No. 11,452,238 and US Patent Application Publication No. 2023/0112841 disclose immersion cooling using certain trans-fluorinated olefins.

Thus, applicants have come to appreciate the need, among the other needs described herein, for thermal management methods and systems which use a heat transfer fluid which is environmentally acceptable (low GWP and low ODP), is non-flammable, has acceptable toxicity, and has excellent electrical insulating properties and has thermal properties that provide effective cooling and/or heating, especially in electronics and semiconductor manufacturing processes that involve relatively high temperatures and/or for use to maintain process conditions in relatively narrow temperature range(s).

### SUMMARY

The present disclosure relates to the compounds trans and cis-1,3,4,4,5,5,5-heptafluoro-3-(trifluoromethyl)pent-1-ene and trans and cis-1,4,4,4-tetrafluoro-3,3-bis(trifluoromethyl)but-1-ene, what are used in methods of heating and/or cooling of electronic components, articles and/or devices during the manufacture and/or operation thereof include providing an electronic component, article or device which is being manufactured and/or being operated for its intended purpose; and transferring heat to and/or from said electronic component, article and/or device during at least a portion of said manufacturing and/or operating process by directly or indirectly transferring heat between said electronic component, article and/or device. Additionally, a refrigerant fluid comprising at least trans-1,3,4,4,5,5,5-heptafluoro-3-(trifluoromethyl)pent-1-ene or trans-1,4,4,4-tetrafluoro-3,3-bis(trifluoromethyl)but-1-ene is disclosed.

In a first embodiment, the present disclosure provides the molecule trans-1,3,4,4,5,5,5-heptafluoro-3-(trifluoromethyl)pent-1-ene.

In a second embodiment, the present disclosure provides the molecule cis-1,3,4,4,5,5,5-heptafluoro-3-(trifluoromethyl)pent-1-ene

In a third embodiment, the present disclosure provides the molecule trans-1,4,4,4-tetrafluoro-2-2-bis(trifluoromethyl)butane.

In a fourth embodiment, the present disclosure provides the molecule cis-1,4,4,4-tetrafluoro-2-2-bis(trifluoromethyl)butane.

In a fifth embodiment, the present disclosure provides methods for synthesizing the trans-1,3,4,4,5,5,5-heptafluoro-3-(trifluoromethyl)pent-1-ene and/or cis-1,3,4,4,5,5,5-heptafluoro-3-(trifluoromethyl)pent-1-ene.

In a sixth embodiment, the present disclosure provides methods for synthesizing trans-1,4,4,4-tetrafluoro-2-2-bis(trifluoromethyl)butane and/or cis-1,4,4,4-tetrafluoro-2-2-bis(trifluoromethyl)butane.

In a seventh embodiment, the present disclosure provides methods of heating and/or cooling of electronic components, articles and/or devices during the manufacture and/or operation thereof with compositions comprising trans-1,3,4,4,5,5,5-heptafluoro-3-(trifluoromethyl)pent-1-ene, cis-1,3,4,4,5,5,5-heptafluoro-3-(trifluoromethyl)pent-1-ene, trans-1,4,4,4-tetrafluoro-2-2-bis(trifluoromethyl)butane, and/or cis-1,4,4,4-tetrafluoro-2-2-bis(trifluoromethyl)butane.

In an eight embodiment, the present disclosure provides heat transfer compositions comprising trans-1,3,4,4,5,5,5-heptafluoro-3-(trifluoromethyl)pent-1-ene, cis-1,3,4,4,5,5,5-heptafluoro-3-(trifluoromethyl)pent-1-ene, trans-1,4,4,4-tetrafluoro-2-2-bis(trifluoromethyl)butane, and/or cis-1,4,4,4-tetrafluoro-2-2-bis(trifluoromethyl)butane and one or more lubricants.

### BRIEF DESCRIPTION OF THE DRAWINGS

The above mentioned and other features of the present invention, and the manner of attaining them, will become more apparent and the invention itself will be better understood by reference to the following description of embodiments of the invention taken in conjunction with the accompanying drawings.

FIG. 1 is a schematic representation of a thermal management system of the present invention.

FIG. 2A is a schematic representation of a first exemplary immersion cooling system according to the present invention.

FIG. 2B is a schematic representation of a second exemplary immersion cooling system according to the present invention.

FIG. 3A is a schematic illustration of a battery thermal management system according to one embodiment of the present invention.

FIG. 3B is a schematic illustration of a second battery thermal management system according to one embodiment of the present invention.

FIG. 4 is a photograph showing a battery thermal management system according to one embodiment of the present invention.

FIG. 5 is a schematic diagram of an exemplary organic Rankine cycle.

FIG. 6 is a schematic diagram of an exemplary heat pump.

FIG. 7 is a schematic diagram of an exemplary secondary loop system.

FIG. 8 is a semi-schematic drawing of one example of a lithium-ion battery cooling system using a composition of the present invention.

FIG. 9 is a semi-schematic drawing of a heat pipe using a heat transfer composition of the present invention.

FIG. 10 is a side cross-sectional view of a conventional wet-etching station.

### DETAILED DESCRIPTION

### I. Definitions

As used herein, a reference to a defined group, such as “Heat Transfer Method 2-3” refers to each method within that group, including wherein a definition number includes a suffix. Thus, reference to “Heat Transfer Method 2-3” includes reference to each Heat Transfer Method 2A, Heat Transfer Method 2B, etc. and Heat Transfer Method 3A, Heat Transfer Method 3B, etc. As another example, “Refrigerant 1-4” refers to each refrigerant within that group, including wherein a definition number includes a suffix. Thus, reference to Refrigerants 1-4 includes reference to Refrigerant 1, including reference to each Refrigerant within the group, such as refrigerant designations 1A1, 1A2, 1A3, etc. as defined by Table 1. “Electronic Device”, and related word forms, means a device, or a component of a device, which is in the process of performing its intended function by receiving, and/or transmitting and/or producing electrical energy and/or electronic signals. Thus, the term “operating electronic device” as used herein includes, for example, a battery which is in the process of providing a source of electrical energy to another component and also a battery which is being charged or recharged, for example.

The term “Heat Transfer Composition” and related word forms means a composition in the form of a fluid (liquid or gas) which is used to transfer heat or energy from one fluid, article or device to another fluid, article or device, and thus includes for example refrigerants, thermal management fluids and working fluids for Rankine cycles, high temperature heat pumps, secondary loop systems, and the like. The heat transfer composition may include each of lubricant (which may also be referred to herein as a dielectric fluid) and a refringent, as well as any other various additives/additional components as desired.

The term “Rankine Cycle” as used herein refers to systems which include: 1) a boiler to change liquid to vapor at high pressure; 2) a turbine to expand the vapor to derive mechanical energy; 3) a condenser to change low pressure exhaust vapor from the turbine to low pressure liquid; and 4) a pump to move condensate liquid back to the boiler at high pressure. Such systems are commonly used for electrical power generation.

When a heat transfer composition is used in thermal management to keep a device or article within a particular temperature range (e.g., in electronic cooling), it is sometimes referred herein as a thermal management fluid.

The component(s) that are present in a heat transfer composition for the purpose of transferring heat (as opposed to, for example, providing lubrication or stabilization) in a heat transfer system (e.g., a vapor compression heat transfer system), that component or combination of components are sometimes referred to herein as a refrigerant.

“Operating Electronic Device”, and related word forms, means a device, or a component of a device, which is in the process of performing its intended function by receiving, and/or transmitting and/or producing electrical energy and/or electronic signals. Thus, the term “operating electronic device” as used herein includes, for example, a battery which is in the process of providing a source of electrical energy to another component and also a battery which is being charged or recharged.

“Thermal contact”, and related forms thereof, includes direct contact with the surface and indirect contact though another body or fluid which facilitates the flow of heat between the surface and the fluid.

“Thermal Conductivity” refers to the breakdown voltage in kV as measured in accordance with ASTM D7896-19.

“Global Warming Potential (“GWP”)” was developed to allow comparisons of the global warming impact of different gases. It is a measure of how much energy the emission of one ton of a gas will absorb over a given period of time, relative to the emission of one ton of carbon dioxide. The larger GWP, the more that a given gas warms the Earth compared to CO2 over that time period. The time period usually used for GWP is 100 years. GWP provides a common measure, which allows analysts to add up emission estimates of different gases.

“Flash Point” refers the lowest temperature at which vapors of the liquid will keep burning after the ignition source is removed as determined in accordance with ASTM D3828-16a.

“Non-flammable” is a measure of whether determines whether a liquid (e.g., a pool of a solvent blend) is nonflammable, flammable, and/or exhibits a flash when an open flame is passed across its surface. This is experimentally determined where a solvent spill is simulated by pouring the solvent blend of interest into a watch glass. The flammability of the blend is characterized throughout the evaporation of the puddle to dryness. In blends with flammable components, the blend may be nonflammable initially, but later exhibit a flash or become flammable due to blend composition shift during evaporation. The watch glass test is a conservative test because no external heat is applied to the watch glass, which is chilled by the evaporating solvent. The cold watch glass acts as a condenser for the vapors of the higher boiling point blend components. In the case where a flammable component is high boiling, its concentration increases throughout the evaporation time, rendering the mixture more flammable than it would be under temperature-controlled blend segregation experiments.

“Acceptable toxicity” means a fluid that has toxicity within acceptable limits. This is experimentally determined by a toxicological screening study to assess in vivo acute oral toxicity at a dosage of 2,000 mg/kg/day and 4-hour acute inhalation toxicity at a concentration of about 20,000 ppm.

“Sensible heat” takes it ordinary meaning, that is, that heat is transferred to or from the refrigerant by causing a temperature change in the refrigerant without the refrigerant changing phase.

“Latent heat” takes it ordinary meaning, that is, that heat is transferred to or from the refrigerant by causing the refrigerant to changing phase.

“Dielectric Constant” means the dielectric constant as measured in accordance with ASTM D150-11 at room temperature at 20 giga hertz (GHz).

“Dielectric Strength” refers to the breakdown voltage in kV as measured in accordance with ASTM D87-13, Procedure A, with the modification that the spacing between the electrodes was 2.54 mm and the rate of rise was 500 V/sec.

As used herein, the singular forms “a”, “an”, and “the” include plural references unless the context clearly dictates otherwise.

As used herein, the recitation of numerical ranges by endpoints includes all numbers subsumed within that range (e.g., 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present invention. At the very least, and not as an attempt to limit the application of the doctrine of equivalents to the scope of the claimed embodiments, each numerical parameter should at least be construed in light of the number of reported significant digits and by applying ordinary rounding techniques.

As used herein, the phrase “within any range encompassed by any two of the foregoing values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

I: Compositions including BuFO.

The present invention provides novel fluorine substituted olefins and methods of heating and/or cooling of electronic components, articles and/or devices during the manufacture and/or operation thereof.

The present invention includes the novel compound 1,3,4,4,5,5,5-heptafluoro-3-(trifluoromethyl)pent-1-ene which in the form of the trans isomer, the cis isomer, or a mixture of the trans isomer with the cis isomer, will be referred to herein as “secBuFO”.

The present invention includes the trans isomer of the novel compound 1,3,4,4,5,5,5-heptafluoro-3-(trifluoromethyl)pent-1-ene, which will be referred to herein as “trans-secBuFO” and which has the structure identified below:

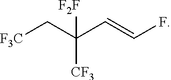

The present invention includes the cis isomer of the novel compound 1,3,4,4,5,5,5-heptafluoro-3-(trifluoromethyl)pent-1-ene, which will be referred to herein as “cis-secBuFO” and which has the structure identified below:

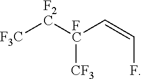

The compound 1,3,4,4,5,5,5-heptafluoro-3-(trifluoromethyl)pent-1-ene may be present in a mixture of the trans and cis isomers at a ratio of trans:cis of about 99:1, about 99.5:0.5, or about 99.9:0.1.

The present invention includes the novel compound 1,4,4,4-tetrafluoro-3,3-bis(trifluoromethyl)but-1-ene which in the form of the trans isomer, the cis isomer, or a mixture of the trans isomer with the cis isomer, will be referred to herein as “tBuFO”.

The present invention includes the trans isomer of the novel compound 1,4,4,4-tetrafluoro-3,3-bis(trifluoromethyl)but-1-ene, which will be referred to herein as “trans-tBuFO” and which has the structure identified below:

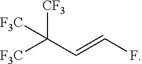 [SMILES: F/C=C/C(C(F)(F)F)(C(F)(F)F)C(F)(F)F]

The present invention includes the cis isomer of the novel compound 1,4,4,4-tetrafluoro-3,3-bis(trifluoromethyl)but-1-ene, which will be referred to herein as “cis-tBuFO” and which has the structure identified below:

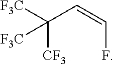 [SMILES: F/C=C\C(C(F)(F)F)(C(F)(F)F)C(F)(F)F]

The compound 1,4,4,4-tetrafluoro-3,3-bis(trifluoromethyl)but-1-ene may be present in a mixture of the trans and cis isomers at a ratio of trans:cis of about 99:1, about 99.5:0.5, or about 99.9:0.1.

The present invention includes refrigerant compositions comprising at least secBuFO. Refrigerant compositions according to this paragraph are sometimes referred to herein for convenience as Refrigerant 1A.

The present invention includes refrigerant compositions comprising at least tBuFO. Refrigerant compositions according to this paragraph are sometimes referred to herein for convenience as Refrigerant 1B.

The present invention includes refrigerant compositions comprising tBuFO and secBuFO. Refrigerant compositions according to this paragraph are sometimes referred to herein for convenience as Refrigerant 1C.

The present invention includes refrigerant compositions comprising at least about 0.01 weight % to less than about 30 weight % secBuFO, as based upon the total weight of the refrigerant composition. Refrigerant compositions according to this paragraph are sometimes referred to herein for convenience as Refrigerant 2A.

The present invention includes refrigerant compositions comprising at least about 0.01 weight % to less than about 30 weight % tBuFO, as based upon the total weight of the refrigerant composition. Refrigerant compositions according to this paragraph are sometimes referred to herein for convenience as Refrigerant 2B.

The present invention includes refrigerant compositions comprising at least about 0.01 weight % to less than or equal to about 30 weight % secBuFO and tBuFO, as based upon the total weight of the refrigerant composition. Refrigerant compositions according to this paragraph are sometimes referred to herein for convenience as Refrigerant 2C.

The present invention includes refrigerant compositions comprising at least about 30 weight % to less than about 70 weight % secBuFO, as based upon the total weight of the refrigerant composition. Refrigerant compositions according to this paragraph are sometimes referred to herein for convenience as Refrigerant 3A.

The present invention includes refrigerant compositions comprising at least about 30 weight % to less than about 70 weight % tBuFO, as based upon the total weight of the refrigerant composition. Refrigerant compositions according to this paragraph are sometimes referred to herein for convenience as Refrigerant 3B.

The present invention includes refrigerant compositions comprising at least about 30 weight % to less than about 70 weight % secBuFO and tBuFO, as based upon the total weight of the refrigerant composition. Refrigerant compositions according to this paragraph are sometimes referred to herein for convenience as Refrigerant 3C.

The present invention includes refrigerant compositions comprising at least about 70 weight % to less than 100 weight % secBuFO, as based upon the total weight of the refrigerant composition. Refrigerant compositions according to this paragraph are sometimes referred to herein for convenience as Refrigerant 4A.

The present invention includes refrigerant compositions comprising at least about 70 weight % to less than 100 weight % tBuFO, as based upon the total weight of the refrigerant composition. Refrigerant compositions according to this paragraph are sometimes referred to herein for convenience as Refrigerant 4B.

The present invention includes refrigerant compositions comprising at least about 70 weight % to less than 100 weight % secBuFO and tBuFo, as based upon the total weight of the refrigerant composition. Refrigerant compositions according to this paragraph are sometimes referred to herein for convenience as Refrigerant 4C.

Table 1 below defines preferred refrigerant compositions contemplated by the present disclosure. It is understood that reference Refrigerants 1-4 include each individual iteration of as described as “refrigerant designation” in Table 1. Specifically, the designation of refrigerant “[#]Letter[#]Letter” indicates certain isomeric configurations of either, or both of the components of the refrigerant composition (e.g., secBuFO and/or tBuFO) as defined in Table 1. For instance, with respect to Refrigerant 1, the refrigerant designation 1A1 in column 1 encompasses the refrigerant 1A (e.g., see BuFO) as defined in column 3, where the secBuFO is the cis configuration, as defined in column 4.

Table 1 also indicate the nature of the refrigerant composition. Here, in the second column, the abbreviations COMP, CEO, and CO are used to identify the nature of the elements of the components of the refrigerant composition. In particular, the designation COMP in the second column indicates that the refrigerant comprises the component of the third column in the isometric configuration of the fourth column at the weight percent range as defined in the fifth column. The designation CEO in the second column indicates that the refrigerant consists essentially of the component of the third column in the isometric configuration of the fourth column at the weight percent range as defined in the fifth column. Finally, designation CO in the second column indicates that the refrigerant consists of the component of the third column in the isometric configuration of the fourth column at the weight percent range as defined in the fifth column.

The weight percent ranges of the fifth column correspond with each of A) NR, which, similar to Table 2, indicates a value that is not required, B) weight percent ranges greater than about 0.01 weight % to less than about 30 wt. percent, C) greater than or equal to about 30 weight percent to less than about 70 weight percent, and D) greater than or equal to about 70 weight percent to less than about 100 wt. percent, each as based upon the total weight of the refrigerant composition.

Therefore, it is meant by the present disclosure, that reference to Refrigerants 1-4 encompass all iteration of Refrigerants 1-4 as described in Table 1 (e.g., 1A1 through 4C9C). It is also intended that in the following table each value for weight percent is understood to be preceded by the term “about.”

TABLE 1
Refrigerant Composition Table

| Refrig- | | | | |
|---|---|---|---|---|
| erant | | | | |
| Desig- | | | Isomeric | Weight % |
| nation | Nature | Component(s) | Configuration(s) | Range |
| 1A1A | COMP | secBuFO | Cis | N/R |
| 1A1B | CEO | secBuFO | Cis | N/R |
| 1A1C | CO | secBuFO | Cis | N/R |
| 1A2A | COMP | secBuFO | Trans | N/R |
| 1A2B | CEO | secBuFO | Trans | N/R |
| 1A2C | CO | secBuFO | Trans | N/R |
| 1A3A | COMP | secBuFO | Trans and Cis | N/R |
| 1A3B | CEO | secBuFO | Trans and Cis | N/R |
| 1A3C | CO | secBuFO | Trans and Cis | N/R |
| 1B1A | COMP | tBuFO | Cis | N/R |
| 1B1B | CEO | tBuFO | Cis | N/R |
| 1B1C | CO | tBuFO | Cis | N/R |
| 1B2A | COMP | tBuFO | Trans | N/R |
| 1B2B | CEO | tBuFO | Trans | N/R |
| 1B2C | CO | tBuFO | Trans | N/R |
| 1B3A | COMP | tBuFO | Trans and Cis | N/R |
| 1B3B | CEO | tBuFO | Trans and Cis | N/R |
| 1B3C | CO | tBuFO | Trans and Cis | N/R |
| 1C1A | COMP | secBuFO & | Cis and Cis | N/R |
| | | tBuFO | | |
| 1C1B | CEO | secBuFO & | Cis and Cis | N/R |
| | | tBuFO | | |
| 1C1C | CO | secBuFO & | Cis and Cis | N/R |
| | | tBuFO | | |
| 1C2A | COMP | secBuFO & | Cis and Trans | N/R |
| | | tBuFO | | |
| 1C2B | CEO | secBuFO & | Cis and Trans | N/R |
| | | tBuFO | | |
| 1C2C | CO | secBuFO & | Cis and Trans | N/R |
| | | tBuFO | | |
| 1C3A | COMP | secBuFO & | Trans and Cis | N/R |
| | | tBuFO | | |
| 1C3B | CEO | secBuFO & | Trans and Cis | N/R |
| | | tBuFO | | |
| 1C3C | CC | secBuFO & | Trans and Cis | N/R |
| | | tBuFO | | |
| 1C4A | COMP | secBuFO & | Trans and Trans | N/R |
| | | tBuFO | | |
| 1C4B | CEO | secBuFO & | Trans and Trans | N/R |
| | | tBuFO | | |
| 1C4C | CO | secBuFO & | Trans and Trans | N/R |
| | | tBuFO | | |
| 1C5A | COMP | secBuFO & | Cis and Trans & | N/R |
| | | tBuFO | Cis | |
| 1C5B | CEO | secBuFO & | Cis and Trans & | N/R |
| | | tBuFO | Cis | |
| 1C5C | CO | secBuFO & | Cis and Trans & | N/R |
| | | tBuFO | Cis | |
| 1C6A | COMP | secBuFO & | Trans and Trans & | N/R |
| | | tBuFO | Cis | |
| 1C6B | CEO | secBuFO & | Trans and Trans & | N/R |
| | | tBuFO | Cis | |
| 1C6C | CO | secBuFO & | Trans and Trans & | N/R |
| | | tBuFO | Cis | |
| 1C7A | COMP | secBuFO & | Trans & Cis and | N/R |
| | | tBuFO | Cis | |
| 1C7B | CEO | secBuFO & | Trans & Cis and | N/R |
| | | tBuFO | Cis | |
| 1C7C | CO | secBuFO & | Trans & Cis and | N/R |
| | | tBuFO | Cis | |
| 1C8A | COMP | secBuFO & | Trans & Cis and | N/R |
| | | tBuFO | Trans | |
| 1C8B | CEO | secBuFO & | Trans & Cis and | N/R |
| | | tBuFO | Trans | |
| 1C8C | CO | secBuFO & | Trans & Cis and | N/R |
| | | tBuFO | Trans | |
| 1C9A | COMP | secBuFO & | Trans & Cis and | N/R |
| | | tBuFO | Trans & Cis | |
| 1C9B | CEO | secBuFO & | Trans & Cis and | N/R |
| | | tBuFO | Trans & Cis | |
| 1C9C | CO | secBuFO & | Trans & Cis and | N/R |
| | | tBuFO | Trans & Cis | |
| 2A1A | COMP | secBuFO | Cis | ≥0.01 wt. % |
| | | | | to <30 wt. % |
| 2A1B | CEO | secBuFO | Cis | ≥0.01 wt. % |
| | | | | to <30 wt. % |
| 2A1C | CO | secBuFO | Cis | ≥0.01 wt. % |
| | | | | to <30 wt. % |
| 2A2A | COMP | secBuFO | Trans | ≥0.01 wt. % |
| | | | | to <30 wt. % |
| 2A2B | CEO | secBuFO | Trans | ≥0.01 wt. % |
| | | | | to <30 wt. % |
| 2A2C | CO | secBuFO | Trans | ≥0.01 wt. % |
| | | | | to <30 wt. % |
| 2A3A | COMP | secBuFO | Trans and Cis | ≥0.01 wt. % |
| | | | | to <30 wt. % |
| 2A3B | CEO | secBuFO | Trans and Cis | ≥0.01 wt. % |
| | | | | to <30 wt. % |
| 2A3C | CO | secBuFO | Trans and Cis | ≥0.01 wt. % |
| | | | | to <30 wt. % |
| 2B1A | COMP | tBuFO | Cis | ≥0.01 wt. % |
| | | | | to <30 wt. % |
| 2B1B | CEO | tBuFO | Cis | ≥0.01 wt. % |
| | | | | to <30 wt. % |
| 2B1C | CC | tBuFO | Cis | ≥0.01 wt. % |
| | | | | to <30 wt. % |
| 2B2A | COMP | tBuFO | Trans | ≥0.01 wt. % |
| | | | | to <30 wt. % |
| 2B2B | CEO | tBuFO | Trans | ≥0.01 wt. % |
| | | | | to <30 wt. % |
| 2B2C | CO | tBuFO | Trans | ≥0.01 wt. % |
| | | | | to <30 wt. % |
| 2B3A | COMP | tBuFO | Trans and Cis | ≥0.01 wt. % |
| | | | | to <30 wt. % |
| 2B3B | CEO | tBuFO | Trans and Cis | ≥0.01 wt. % |
| | | | | to <30 wt. % |
| 2B3C | CO | tBuFO | Trans and Cis | ≥0.01 wt. % |
| | | | | to <30 wt. % |
| 2C1A | COMP | secBuFO & | Cis and Cis | ≥0.01 wt. % |
| | | tBuFO | | to <30 wt. % |
| 2C1B | CEO | secBuFO & | Cis and Cis | ≥0.01 wt. % |
| | | tBuFO | | to <30 wt. % |
| 2C1C | CC | secBuFO & | Cis and Cis | ≥0.01 wt. % |
| | | tBuFO | | to <30 wt. % |
| 2C2A | COMP | secBuFO & | Cis and Trans | ≥0.01 wt. % |
| | | tBuFO | | to <30 wt. % |
| 2C2B | CEO | secBuFO & | Cis and Trans | ≥0.01 wt. % |
| | | tBuFO | | to <30 wt. % |
| 2C2C | CC | secBuFO & | Cis and Trans | ≥0.01 wt. % |
| | | tBuFO | | to <30 wt. % |
| 2C3A | COMP | secBuFO & | Trans and Cis | ≥0.01 wt. % |
| | | tBuFO | | to <30 wt. % |
| 2C3B | CEO | secBuFO & | Trans and Cis | ≥0.01 wt. % |
| | | tBuFO | | to <30 wt. % |
| 2C3C | CO | secBuFO & | Trans and Cis | ≥0.01 wt. % |
| | | tBuFO | | to <30 wt. % |
| 2C4A | COMP | secBuFO & | Trans and Trans | ≥0.01 wt. % |
| | | tBuFO | | to <30 wt. % |
| 2C4B | CEO | secBuFO & | Trans and Trans | ≥0.01 wt. % |
| | | tBuFO | | to <30 wt. % |
| 2C4C | CO | secBuFO & | Trans and Trans | ≥0.01 wt. % |
| | | tBuFO | | to <30 wt. % |
| 2C5A | COMP | secBuFO & | Cis and Trans & | ≥0.01 wt. % |
| | | tBuFO | Cis | to <30 wt. % |
| 2C5B | CEO | secBuFO & | Cis and Trans & | ≥0.01 wt. % |
| | | tBuFO | Cis | to <30 wt. % |
| 2C5C | CO | secBuFO & | Cis and Trans & | ≥0.01 wt. % |
| | | tBuFO | Cis | to <30 wt. % |
| 2C6A | COMP | secBuFO & | Trans and Trans & | ≥0.01 wt. % |
| | | tBuFO | Cis | to <30 wt. % |
| 2C6B | CEO | secBuFO & | Trans and Trans & | ≥0.01 wt. % |
| | | tBuFO | Cis | to <30 wt. % |
| 2C6C | CO | secBuFO & | Trans and Trans & | ≥0.01 wt. % |
| | | tBuFO | Cis | to <30 wt. % |
| 2C7A | COMP | secBuFO & | Trans & Cis and | ≥0.01 wt. % |
| | | tBuFO | Cis | to <30 wt. % |
| 2C7B | CEO | secBuFO & | Trans & Cis and | ≥0.01 wt. % |
| | | tBuFO | Cis | to <30 wt. % |
| 2C7C | CO | secBuFO & | Trans & Cis and | ≥0.01 wt. % |
| | | tBuFO | Cis | to <30 wt. % |
| 2C8A | COMP | secBuFO & | Trans & Cis and | ≥0.01 wt. % |
| | | tBuFO | Trans | to <30 wt. % |
| 2C8B | CEO | secBuFO & | Trans & Cis and | ≥0.01 wt. % |
| | | tBuFO | Trans | to <30 wt. % |
| 2C8C | CO | secBuFO & | Trans & Cis and | ≥0.01 wt. % |
| | | tBuFO | Trans | to <30 wt. % |
| 2C9A | COMP | secBuFO & | Trans & Cis and | ≥0.01 wt. % |
| | | tBuFO | Trans & Cis | to <30 wt. % |
| 2C9B | CEO | secBuFO & | Trans & Cis and | ≥0.01 wt. % |
| | | tBuFO | Trans & Cis | to <30 wt. % |
| 2C9C | CO | secBuFO & | Trans & Cis and | ≥0.01 wt. % |
| | | tBuFO | Trans & Cis | to <30 wt. % |
| 3A1A | COMP | secBuFO | Cis | ≥30 wt. % |
| | | | | to <70 wt. % |
| 3A1B | CEO | secBuFO | Cis | ≥30 wt. % |
| | | | | to <70 wt. % |
| 3A1C | CO | secBuFO | Cis | ≥30 wt. % |
| | | | | to <70 wt. % |
| 3A2A | COMP | secBuFO | Trans | ≥30 wt. % |
| | | | | to <70 wt. % |
| 3A2B | CEO | secBuFO | Trans | ≥30 wt. % |
| | | | | to <70 wt. % |
| 3A2C | CO | secBuFO | Trans | ≥30 wt. % |
| | | | | to <70 wt. % |
| 3A3A | COMP | secBuFO | Trans and Cis | ≥30 wt. % |
| | | | | to <70 wt. % |
| 3A3B | CEO | secBuFO | Trans and Cis | ≥30 wt. % |
| | | | | to <70 wt. % |
| 3A3C | CC | secBuFO | Trans and Cis | ≥30 wt. % |
| | | | | to <70 wt. % |
| 3B1A | COMP | tBuFO | Cis | ≥30 wt. % |
| | | | | to <70 wt. % |
| 3B1B | CEO | tBuFO | Cis | ≥30 wt. % |
| | | | | to <70 wt. % |
| 3B1C | CC | tBuFO | Cis | ≥30 wt. % |
| | | | | to <70 wt. % |
| 3B2A | COMP | tBuFO | Trans | ≥30 wt. % |
| | | | | to <70 wt. % |
| 3B2B | CEO | tBuFO | Trans | ≥30 wt. % |
| | | | | to <70 wt. % |
| 3B2C | CC | tBuFO | Trans | ≥30 wt. % |
| | | | | to <70 wt. % |
| 3B3A | COMP | tBuFO | Trans and Cis | ≥30 wt. % |
| | | | | to <70 wt. % |
| 3B3B | CEO | tBuFO | Trans and Cis | ≥30 wt. % |
| | | | | to <70 wt. % |
| 3B3C | CO | tBuFO | Trans and Cis | ≥30 wt. % |
| | | | | to <70 wt. % |
| 3C1A | COMP | secBuFO & | Cis and Cis | ≥30 wt. % |
| | | tBuFO | | to <70 wt. % |
| 3C1B | CEO | secBuFO & | Cis and Cis | ≥30 wt. % |
| | | tBuFO | | to <70 wt. % |
| 3C1C | CO | secBuFO & | Cis and Cis | ≥30 wt. % |
| | | tBuFO | | to <70 wt. % |
| 3C2A | COMP | secBuFO & | Cis and Trans | ≥30 wt. % |
| | | tBuFO | | to <70 wt. % |
| 3C2B | CEO | secBuFO & | Cis and Trans | ≥30 wt. % |
| | | tBuFO | | to <70 wt. % |
| 3C2C | CO | secBuFO & | Cis and Trans | ≥30 wt. % |
| | | tBuFO | | to <70 wt. % |
| 3C3A | COMP | secBuFO & | Trans and Cis | ≥30 wt. % |
| | | tBuFO | | to <70 wt. % |
| 3C3B | CEO | secBuFO & | Trans and Cis | ≥30 wt. % |
| | | tBuFO | | to <70 wt. % |
| 3C3C | CO | secBuFO & | Trans and Cis | ≥30 wt. % |
| | | tBuFO | | to <70 wt. % |
| 3C4A | COMP | secBuFO & | Trans and Trans | ≥30 wt. % |
| | | tBuFO | | to <70 wt. % |
| 3C4B | CEO | secBuFO & | Trans and Trans | ≥30 wt. % |
| | | tBuFO | | to <70 wt. % |
| 3C4C | CO | secBuFO & | Trans and Trans | ≥30 wt. % |
| | | tBuFO | | to <70 wt. % |
| 3C5A | COMP | secBuFO & | Cis and Trans & | ≥30 wt. % |
| | | tBuFO | Cis | to <70 wt. % |
| 3C5B | CEO | secBuFO & | Cis and Trans & | ≥30 wt. % |
| | | tBuFO | Cis | to <70 wt. % |
| 3C5C | CC | secBuFO & | Cis and Trans & | ≥30 wt. % |
| | | tBuFO | Cis | to <70 wt. % |
| 3C6A | COMP | secBuFO & | Trans and Trans & | ≥30 wt. % |
| | | tBuFO | Cis | to <70 wt. % |
| 3C6B | CEO | secBuFO & | Trans and Trans & | ≥30 wt. % |
| | | tBuFO | Cis | to <70 wt. % |
| 3C6C | CO | secBuFO & | Trans and Trans & | ≥30 wt. % |
| | | tBuFO | Cis | to <70 wt. % |
| 3C7A | COMP | secBuFO & | Trans & Cis and | ≥30 wt. % |
| | | tBuFO | Cis | to <70 wt. % |
| 3C7B | CEO | secBuFO & | Trans & Cis and | ≥30 wt. % |
| | | tBuFO | Cis | to <70 wt. % |
| 3C7C | CO | secBuFO & | Trans & Cis and | ≥30 wt. % |
| | | tBuFO | Cis | to <70 wt. % |
| 3C8A | COMP | secBuFO & | Trans & Cis and | ≥30 wt. % |
| | | tBuFO | Trans | to <70 wt. % |
| 3C8B | CEO | secBuFO & | Trans & Cis and | ≥30 wt. % |
| | | tBuFO | Trans | to <70 wt. % |
| 3C8C | CO | secBuFO & | Trans & Cis and | ≥30 wt. % |
| | | tBuFO | Trans | to <70 wt. % |
| 3C9A | COMP | secBuFO & | Trans & Cis and | ≥30 wt. % |
| | | tBuFO | Trans & Cis | to <70 wt. % |
| 3C9B | CEO | secBuFO & | Trans & Cis and | ≥30 wt. % |
| | | tBuFO | Trans & Cis | to <70 wt. % |
| 3C9C | CO | secBuFO & | Trans & Cis and | ≥30 wt. % |
| | | tBuFO | Trans & Cis | to <70 wt. % |
| 4A1A | COMP | secBuFO | Cis | ≥70 wt. % |
| | | | | to <100 wt. % |
| 4A1B | CEO | secBuFO | Cis | ≥70 wt. % |
| | | | | to <100 wt. % |
| 4A1C | CO | secBuFO | Cis | ≥70 wt. % |
| | | | | to <100 wt. % |
| 4A2A | COMP | secBuFO | Trans | ≥70 wt. % |
| | | | | to <100 wt. % |
| 4A2B | CEO | secBuFO | Trans | ≥70 wt. % |
| | | | | to <100 wt. % |
| 4A2C | CO | secBuFO | Trans | ≥70 wt. % |
| | | | | to <100 wt. % |
| 4A3A | COMP | secBuFO | Trans and Cis | ≥70 wt. % |
| | | | | to <100 wt. % |
| 4A3B | CEO | secBuFO | Trans and Cis | ≥70 wt. % |
| | | | | to <100 wt. % |
| 4A3C | CO | secBuFO | Trans and Cis | ≥70 wt. % |
| | | | | to <100 wt. % |
| 4B1A | COMP | tBuFO | Cis | ≥70 wt. % |
| | | | | to <100 wt. % |
| 4B1B | CEO | tBuFO | Cis | ≥70 wt. % |
| | | | | to <100 wt. % |
| 4B1C | CO | tBuFO | Cis | ≥70 wt. % |
| | | | | to <100 wt. % |
| 4B2A | COMP | tBuFO | Trans | ≥70 wt. % |
| | | | | to <100 wt. % |
| 4B2B | CEO | tBuFO | Trans | ≥70 wt. % |
| | | | | to <100 wt. % |
| 4B2C | CO | tBuFO | Trans | ≥70 wt. % |
| | | | | to <100 wt. % |
| 4B3A | COMP | tBuFO | Trans and Cis | ≥70 wt. % |
| | | | | to <100 wt. % |
| 4B3B | CEO | tBuFO | Trans and Cis | ≥70 wt. % |
| | | | | to <100 wt. % |
| 4B3C | CO | tBuFO | Trans and Cis | ≥70 wt. % |
| | | | | to <100 wt. % |
| 4C1A | COMP | secBuFO & | Cis and Cis | ≥70 wt. % |
| | | tBuFO | | to <100 wt. % |
| 4C1B | CEO | secBuFO & | Cis and Cis | ≥70 wt. % |
| | | tBuFO | | to <100 wt. % |
| 4C1C | CO | secBuFO & | Cis and Cis | ≥70 wt. % |
| | | tBuFO | | to <100 wt. % |
| 4C2A | COMP | secBuFO & | Cis and Trans | ≥70 wt. % |
| | | tBuFO | | to <100 wt. % |
| 4C2B | CEO | secBuFO & | Cis and Trans | ≥70 wt. % |
| | | tBuFO | | to <100 wt. % |
| 4C2C | CO | secBuFO & | Cis and Trans | ≥70 wt. % |
| | | tBuFO | | to <100 wt. % |
| 4C3A | COMP | secBuFO & | Trans and Cis | ≥70 wt. % |
| | | tBuFO | | to <100 wt. % |
| 4C3B | CEO | secBuFO & | Trans and Cis | ≥70 wt. % |
| | | tBuFO | | to <100 wt. % |
| 4C3C | CO | secBuFO & | Trans and Cis | ≥70 wt. % |
| | | tBuFO | | to <100 wt. % |
| 4C4A | COMP | secBuFO & | Trans and Trans | ≥70 wt. % |
| | | tBuFO | | to <100 wt. % |
| 4C4B | CEO | secBuFO & | Trans and Trans | ≥70 wt. % |
| | | tBuFO | | to <100 wt. % |
| 4C4C | CO | secBuFO & | Trans and Trans | ≥70 wt. % |
| | | tBuFO | | to <100 wt. % |
| 4C5A | COMP | secBuFO & | Cis and Trans & | ≥70 wt. % |
| | | tBuFO | Cis | to <100 wt. % |
| 4C5B | CEO | secBuFO & | Cis and Trans & | ≥70 wt. % |
| | | tBuFO | Cis | to <100 wt. % |
| 4C5C | CO | secBuFO & | Cis and Trans & | ≥70 wt. % |
| | | tBuFO | Cis | to <100 wt. % |
| 4C6A | COMP | secBuFO & | Trans and Trans & | ≥70 wt. % |
| | | tBuFO | Cis | to <100 wt. % |
| 4C6B | CEO | secBuFO & | Trans and Trans & | ≥70 wt. % |
| | | tBuFO | Cis | to <100 wt. % |
| 4C6C | CO | secBuFO & | Trans and Trans & | ≥70 wt. % |
| | | tBuFO | Cis | to <100 wt. % |
| 4C7A | COMP | secBuFO & | Trans & Cis and | ≥70 wt. % |
| | | tBuFO | Cis | to <100 wt. % |
| 4C7B | CEO | secBuFO & | Trans & Cis and | ≥70 wt. % |
| | | tBuFO | Cis | to <100 wt. % |
| 4C7C | CO | secBuFO & | Trans & Cis and | ≥70 wt. % |
| | | tBuFO | Cis | to <100 wt. % |
| 4C8A | COMP | secBuFO & | Trans & Cis and | ≥70 wt. % |
| | | tBuFO | Trans | to <100 wt. % |
| 4C8B | CEO | secBuFO & | Trans & Cis and | ≥70 wt. % |
| | | tBuFO | Trans | to <100 wt. % |
| 4C8C | CO | secBuFO & | Trans & Cis and | ≥70 wt. % |
| | | tBuFO | Trans | to <100 wt. % |
| 4C9A | COMP | secBuFO & | Trans & Cis and | ≥70 wt. % |
| | | tBuFO | Trans & Cis | to <100 wt. % |
| 4C9B | CEO | secBuFO & | Trans & Cis and | ≥70 wt. % |
| | | tBuFO | Trans & Cis | to <100 wt. % |
| 4C9C | CO | secBuFO & | Trans & Cis and | ≥70 wt. % |
| | | tBuFO | Trans & Cis | to <100 wt. % |

Table 2 below defines preferred heat transfer compositions contemplated by the present disclosure, which either comprise, consist essentially of, or consist of the refrigerants as described with reference to Table 1 above (e.g., including certain isomeric configuration of secBuFO and/or tBuFO) as well as a selection of a lubricant.

Here, the lubricant, which may also be a dielectric fluid, may be selected from any suitable fluid possessing electrically insulative, thermally conductive, lubricating, and/or moisture preventive properties, such as any one of, or combination of, mineral oils, synthetic dielectric oils in including polyalkylene glycol (PAG), vegetable dielectric oils, aromatic hydrocarbons such as alkylbenzenes, alkyldiphenylethanes, alkylnaphthalenes, methylpolyarylmethanes (and combination of the foregoing), Polyvinyl Ether oils (PVE), poly(α-)olefin oils, polyol esters oil, and paraffinic oils. Preferred dielectric fluids including poly(α-)olefin oils (PAO); polyol esters oil (POE), and Parraficinc oils, however, the disclosure is nonlimiting. Hereinafter, the designation Heat Transfer Composition A encompasses any one of the heat transfer compositions A1-A540; Heat Transfer Composition B encompasses any one of the heat transfer compositions B1-B540; Heat Transfer Composition C encompasses any one of the heat transfer compositions of C1-C540, and Heat Transfer Composition C encompasses any one of the heat transfer compositions of D1-D540.

Specifically, the first column of Table 2 below indicates the heat transfer composition number (e.g., A1-A540; B1-B540; C1-C540; and D1-540). The second column indicates the refrigerant used in combination with the given lubricant of column 4, which are the same refrigerant designations as defined in Table 1. In the third column, the abbreviations COMP, CEO, and CO are used to identify the nature of the elements of the components of the heat transfer composition. In particular, the designation COMP in the third column indicates that the heat transfer composition comprises the dielectric fluid in the fourth column and the refrigerant of the second column. The designation CEO in the third column indicates that the heat transfer composition consists essentially of the dielectric fluid in the fourth column and the refrigerant of the second column. Finally, designation CO in the third column indicates that the heat transfer composition consists of the dielectric fluid in the fourth column and the refrigerant of the second column.

TABLE 2
Heat Transfer Composition Table

| | | | |
|---|---|---|---|
| Heat Transfer | Refrigerant | | |
| Composition | Designation | Nature | Lubricant |

Poly(α-)olefin (PAO) Lubricant

| | | | |
|---|---|---|---|
| A1 | 1A1A | COMP | PAO |
| A2 | 1A1B | COMP | PAO |
| A3 | 1A1C | COMP | PAO |
| A4 | 1A2A | COMP | PAO |
| A5 | 1A2B | COMP | PAO |
| A6 | 1A2C | COMP | PAO |
| A7 | 1A3A | COMP | PAO |
| A8 | 1A3B | COMP | PAO |
| A9 | 1A3C | COMP | PAO |
| A10 | 1B1A | COMP | PAO |
| A11 | 1B1B | COMP | PAO |
| A12 | 1B1C | COMP | PAO |
| A13 | 1B2A | COMP | PAO |
| A14 | 1B2B | COMP | PAO |
| A15 | 1B2C | COMP | PAO |
| A16 | 1B3A | COMP | PAO |
| A17 | 1B3B | COMP | PAO |
| A18 | 1B3C | COMP | PAO |
| A19 | 1C1A | COMP | PAO |
| A20 | 1C1B | COMP | PAO |
| A21 | 1C1C | COMP | PAO |
| A22 | 1C2A | COMP | PAO |
| A23 | 1C2B | COMP | PAO |
| A24 | 1C2C | COMP | PAO |
| A25 | 1C3A | COMP | PAO |
| A26 | 1C3B | COMP | PAO |
| A27 | 1C3C | COMP | PAO |
| A28 | 1C4A | COMP | PAO |
| A29 | 1C4B | COMP | PAO |
| A30 | 1C4C | COMP | PAO |
| A31 | 1C5A | COMP | PAO |
| A32 | 1C5B | COMP | PAO |
| A33 | 1C5C | COMP | PAO |
| A34 | 1C6A | COMP | PAO |
| A35 | 1C6B | COMP | PAO |
| A36 | 1C6C | COMP | PAO |
| A37 | 1C7A | COMP | PAO |
| A38 | 1C7B | COMP | PAO |
| A39 | 1C7C | COMP | PAO |
| A40 | 1C8A | COMP | PAO |
| A41 | 1C8B | COMP | PAO |
| A42 | 1C8C | COMP | PAO |
| A43 | 1C9A | COMP | PAO |
| A44 | 1C9B | COMP | PAO |
| A45 | 1C9C | COMP | PAO |
| A46 | 1A1A | CEO | PAO |
| A47 | 1A1B | CEO | PAO |
| A48 | 1A1C | CEO | PAO |
| A49 | 1A2A | CEO | PAO |
| A50 | 1A2B | CEO | PAO |
| A51 | 1A2C | CEO | PAO |
| A52 | 1A3A | CEO | PAO |
| A53 | 1A3B | CEO | PAO |
| A54 | 1A3C | CEO | PAO |
| A55 | 1B1A | CEO | PAO |
| A56 | 1B1B | CEO | PAO |
| A57 | 1B1C | CEO | PAO |
| A58 | 1B2A | CEO | PAO |
| A59 | 1B2B | CEO | PAO |
| A60 | 1B2C | CEO | PAO |
| A61 | 1B3A | CEO | PAO |
| A62 | 1B3B | CEO | PAO |
| A63 | 1B3C | CEO | PAO |
| A64 | 1C1A | CEO | PAO |
| A65 | 1C1B | CEO | PAO |
| A66 | 1C1C | CEO | PAO |
| A67 | 1C2A | CEO | PAO |
| A68 | 1C2B | CEO | PAO |
| A69 | 1C2C | CEO | PAO |
| A70 | 1C3A | CEO | PAO |
| A71 | 1C3B | CEO | PAO |
| A72 | 1C3C | CEO | PAO |
| A73 | 1C4A | CEO | PAO |
| A74 | 1C4B | CEO | PAO |
| A75 | 1C4C | CEO | PAO |
| A76 | 1C5A | CEO | PAO |
| A77 | 1C5B | CEO | PAO |
| A78 | 1C5C | CEO | PAO |
| A79 | 1C6A | CEO | PAO |
| A80 | 1C6B | CEO | PAO |
| A81 | 1C6C | CEO | PAO |
| A82 | 1C7A | CEO | PAO |
| A83 | 1C7B | CEO | PAO |
| A84 | 1C7C | CEO | PAO |
| A85 | 1C8A | CEO | PAO |
| A86 | 1C8B | CEO | PAO |
| A87 | 1C8C | CEO | PAO |
| A88 | 1C9A | CEO | PAO |
| A89 | 1C9B | CEO | PAO |
| A90 | 1C9C | CEO | PAO |
| A91 | 1A1A | CO | PAO |
| A92 | 1A1B | CO | PAO |
| A93 | 1A1C | CO | PAO |
| A94 | 1A2A | CO | PAO |
| A95 | 1A2B | CO | PAO |
| A96 | 1A2C | CO | PAO |
| A97 | 1A3A | CO | PAO |
| A98 | 1A3B | CO | PAO |
| A99 | 1A3C | CO | PAO |
| A100 | 1B1A | CO | PAO |
| A101 | 1B1B | CO | PAO |
| A102 | 1B1C | CO | PAO |
| A103 | 1B2A | CO | PAO |
| A104 | 1B2B | CO | PAO |
| A105 | 1B2C | CO | PAO |
| A106 | 1B3A | CO | PAO |
| A107 | 1B3B | CO | PAO |
| A108 | 1B3C | CO | PAO |
| A109 | 1C1A | CO | PAO |
| A110 | 1C1B | CO | PAO |
| A111 | 1C1C | CO | PAO |
| A112 | 1C2A | CO | PAO |
| A113 | 1C2B | CO | PAO |
| A114 | 1C2C | CO | PAO |
| A115 | 1C3A | CO | PAO |
| A116 | 1C3B | CO | PAO |
| A117 | 1C3C | CO | PAO |
| A118 | 1C4A | CO | PAO |
| A119 | 1C4B | CO | PAO |
| A120 | 1C4C | CO | PAO |
| A121 | 1C5A | CO | PAO |
| A122 | 1C5B | CO | PAO |
| A123 | 1C5C | CO | PAO |
| A124 | 1C6A | CO | PAO |
| A125 | 1C6B | CO | PAO |
| A126 | 1C6C | CO | PAO |
| A127 | 1C7A | CO | PAO |
| A128 | 1C7B | CO | PAO |
| A129 | 1C7C | CO | PAO |
| A130 | 1C8A | CO | PAO |
| A131 | 1C8B | CO | PAO |
| A132 | 1C8C | CO | PAO |
| A133 | 1C9A | CO | PAO |
| A134 | 1C9B | CO | PAO |
| A135 | 1C9C | CO | PAO |
| A136 | 2A1A | COMP | PAO |
| A137 | 2A1B | COMP | PAO |
| A138 | 2A1C | COMP | PAO |
| A139 | 2A2A | COMP | PAO |
| A140 | 2A2B | COMP | PAO |
| A141 | 2A2C | COMP | PAO |
| A142 | 2A3A | COMP | PAO |
| A143 | 2A3B | COMP | PAO |
| A144 | 2A3C | COMP | PAO |
| A145 | 2B1A | COMP | PAO |
| A146 | 2B1B | COMP | PAO |
| A147 | 2B1C | COMP | PAO |
| A148 | 2B2A | COMP | PAO |
| A149 | 2B2B | COMP | PAO |
| A150 | 2B2C | COMP | PAO |
| A151 | 2B3A | COMP | PAO |
| A152 | 2B3B | COMP | PAO |
| A153 | 2B3C | COMP | PAO |
| A154 | 2C1A | COMP | PAO |
| A155 | 2C1B | COMP | PAO |
| A156 | 2C1C | COMP | PAO |
| A157 | 2C2A | COMP | PAO |
| A158 | 2C2B | COMP | PAO |
| A159 | 2C2C | COMP | PAO |
| A160 | 2C3A | COMP | PAO |
| A161 | 2C3B | COMP | PAO |
| A162 | 2C3C | COMP | PAO |
| A163 | 2C4A | COMP | PAO |
| A164 | 2C4B | COMP | PAO |
| A165 | 2C4C | COMP | PAO |
| A166 | 2C5A | COMP | PAO |
| A167 | 2C5B | COMP | PAO |
| A168 | 2C5C | COMP | PAO |
| A169 | 2C6A | COMP | PAO |
| A170 | 2C6B | COMP | PAO |
| A171 | 2C6C | COMP | PAO |
| A172 | 2C7A | COMP | PAO |
| A173 | 2C7B | COMP | PAO |
| A174 | 2C7C | COMP | PAO |
| A175 | 2C8A | COMP | PAO |
| A176 | 2C8B | COMP | PAO |
| A177 | 2C8C | COMP | PAO |
| A178 | 2C9A | COMP | PAO |
| A179 | 2C9B | COMP | PAO |
| A180 | 2C9C | COMP | PAO |
| A181 | 2A1A | CEO | PAO |
| A182 | 2A1B | CEO | PAO |
| A183 | 2A1C | CEO | PAO |
| A184 | 2A2A | CEO | PAO |
| A185 | 2A2B | CEO | PAO |
| A186 | 2A2C | CEO | PAO |
| A187 | 2A3A | CEO | PAO |
| A188 | 2A3B | CEO | PAO |
| A189 | 2A3C | CEO | PAO |
| A190 | 2B1A | CEO | PAO |
| A191 | 2B1B | CEO | PAO |
| A192 | 2B1C | CEO | PAO |
| A193 | 2B2A | CEO | PAO |
| A194 | 2B2B | CEO | PAO |
| A195 | 2B2C | CEO | PAO |
| A196 | 2B3A | CEO | PAO |
| A197 | 2B3B | CEO | PAO |
| A198 | 2B3C | CEO | PAO |
| A199 | 2C1A | CEO | PAO |
| A200 | 2C1B | CEO | PAO |
| A201 | 2C1C | CEO | PAO |
| A202 | 2C2A | CEO | PAO |
| A203 | 2C2B | CEO | PAO |
| A204 | 2C2C | CEO | PAO |
| A205 | 2C3A | CEO | PAO |
| A206 | 2C3B | CEO | PAO |
| A207 | 2C3C | CEO | PAO |
| A208 | 2C4A | CEO | PAO |
| A209 | 2C4B | CEO | PAO |
| A210 | 2C4C | CEO | PAO |
| A211 | 2C5A | CEO | PAO |
| A212 | 2C5B | CEO | PAO |
| A213 | 2C5C | CEO | PAO |
| A214 | 2C6A | CEO | PAO |
| A215 | 2C6B | CEO | PAO |
| A216 | 2C6C | CEO | PAO |
| A217 | 2C7A | CEO | PAO |
| A218 | 2C7B | CEO | PAO |
| A219 | 2C7C | CEO | PAO |
| A220 | 2C8A | CEO | PAO |
| A221 | 2C8B | CEO | PAO |
| A222 | 2C8C | CEO | PAO |
| A223 | 2C9A | CEO | PAO |
| A224 | 2C9B | CEO | PAO |
| A225 | 2C9C | CEO | PAO |
| A226 | 2A1A | CO | PAO |
| A227 | 2A1B | CO | PAO |
| A228 | 2A1C | CO | PAO |
| A229 | 2A2A | CO | PAO |
| A230 | 2A2B | CO | PAO |
| A231 | 2A2C | CO | PAO |
| A232 | 2A3A | CO | PAO |
| A233 | 2A3B | CO | PAO |
| A234 | 2A3C | CO | PAO |
| A235 | 2B1A | CO | PAO |
| A236 | 2B1B | CO | PAO |
| A237 | 2B1C | CO | PAO |
| A238 | 2B2A | CO | PAO |
| A239 | 2B2B | CO | PAO |
| A240 | 2B2C | CO | PAO |
| A241 | 2B3A | CO | PAO |
| A242 | 2B3B | CO | PAO |
| A243 | 2B3C | CO | PAO |
| A244 | 2C1A | CO | PAO |
| A245 | 2C1B | CO | PAO |
| A246 | 2C1C | CO | PAO |
| A247 | 2C2A | CO | PAO |
| A248 | 2C2B | CO | PAO |
| A249 | 2C2C | CO | PAO |
| A250 | 2C3A | CO | PAO |
| A251 | 2C3B | CO | PAO |
| A252 | 2C3C | CO | PAO |
| A253 | 2C4A | CO | PAO |
| A254 | 2C4B | CO | PAO |
| A255 | 2C4C | CO | PAO |
| A256 | 2C5A | CO | PAO |
| A257 | 2C5B | CO | PAO |
| A258 | 2C5C | CO | PAO |
| A259 | 2C6A | CO | PAO |
| A260 | 2C6B | CO | PAO |
| A261 | 2C6C | CO | PAO |
| A262 | 2C7A | CO | PAO |
| A263 | 2C7B | CO | PAO |
| A264 | 2C7C | CO | PAO |
| A265 | 2C8A | CO | PAO |
| A266 | 2C8B | CO | PAO |
| A267 | 2C8C | CO | PAO |
| A268 | 2C9A | CO | PAO |
| A269 | 2C9B | CO | PAO |
| A270 | 2C9C | CO | PAO |
| A271 | 3A1A | COMP | PAO |
| A272 | 3A1B | COMP | PAO |
| A273 | 3A1C | COMP | PAO |
| A274 | 3A2A | COMP | PAO |
| A275 | 3A2B | COMP | PAO |
| A276 | 3A2C | COMP | PAO |
| A277 | 3A3A | COMP | PAO |
| A278 | 3A3B | COMP | PAO |
| A279 | 3A3C | COMP | PAO |
| A280 | 3B1A | COMP | PAO |
| A281 | 3B1B | COMP | PAO |
| A282 | 3B1C | COMP | PAO |
| A283 | 3B2A | COMP | PAO |
| A284 | 3B2B | COMP | PAO |
| A285 | 3B2C | COMP | PAO |
| A286 | 3B3A | COMP | PAO |
| A287 | 3B3B | COMP | PAO |
| A288 | 3B3C | COMP | PAO |
| A289 | 3C1A | COMP | PAO |
| A290 | 3C1B | COMP | PAO |
| A291 | 3C1C | COMP | PAO |
| A292 | 3C2A | COMP | PAO |
| A293 | 3C2B | COMP | PAO |
| A294 | 3C2C | COMP | PAO |
| A295 | 3C3A | COMP | PAO |
| A296 | 3C3B | COMP | PAO |
| A297 | 3C3C | COMP | PAO |
| A298 | 3C4A | COMP | PAO |
| A299 | 3C4B | COMP | PAO |
| A300 | 3C4C | COMP | PAO |
| A301 | 3C5A | COMP | PAO |
| A302 | 3C5B | COMP | PAO |
| A303 | 3C5C | COMP | PAO |
| A304 | 3C6A | COMP | PAO |
| A305 | 3C6B | COMP | PAO |
| A306 | 3C6C | COMP | PAO |
| A307 | 3C7A | COMP | PAO |
| A308 | 3C7B | COMP | PAO |
| A309 | 3C7C | COMP | PAO |
| A310 | 3C8A | COMP | PAO |
| A311 | 3C8B | COMP | PAO |
| A312 | 3C8C | COMP | PAO |
| A313 | 3C9A | COMP | PAO |
| A314 | 3C9B | COMP | PAO |
| A315 | 3C9C | COMP | PAO |
| A316 | 3A1A | CEO | PAO |
| A317 | 3A1B | CEO | PAO |
| A318 | 3A1C | CEO | PAO |
| A319 | 3A2A | CEO | PAO |
| A320 | 3A2B | CEO | PAO |
| A321 | 3A2C | CEO | PAO |
| A322 | 3A3A | CEO | PAO |
| A323 | 3A3B | CEO | PAO |
| A324 | 3A3C | CEO | PAO |
| A325 | 3B1A | CEO | PAO |
| A326 | 3B1B | CEO | PAO |
| A327 | 3B1C | CEO | PAO |
| A328 | 3B2A | CEO | PAO |
| A329 | 3B2B | CEO | PAO |
| A330 | 3B2C | CEO | PAO |
| A331 | 3B3A | CEO | PAO |
| A332 | 3B3B | CEO | PAO |
| A333 | 3B3C | CEO | PAO |
| A334 | 3C1A | CEO | PAO |
| A335 | 3C1B | CEO | PAO |
| A336 | 3C1C | CEO | PAO |
| A337 | 3C2A | CEO | PAO |
| A338 | 3C2B | CEO | PAO |
| A339 | 3C2C | CEO | PAO |
| A340 | 3C3A | CEO | PAO |
| A341 | 3C3B | CEO | PAO |
| A342 | 3C3C | CEO | PAO |
| A343 | 3C4A | CEO | PAO |
| A344 | 3C4B | CEO | PAO |
| A345 | 3C4C | CEO | PAO |
| A346 | 3C5A | CEO | PAO |
| A347 | 3C5B | CEO | PAO |
| A348 | 3C5C | CEO | PAO |
| A349 | 3C6A | CEO | PAO |
| A350 | 3C6B | CEO | PAO |
| A351 | 3C6C | CEO | PAO |
| A352 | 3C7A | CEO | PAO |
| A353 | 3C7B | CEO | PAO |
| A354 | 3C7C | CEO | PAO |
| A355 | 3C8A | CEO | PAO |
| A356 | 3C8B | CEO | PAO |
| A357 | 3C8C | CEO | PAO |
| A358 | 3C9A | CEO | PAO |
| A359 | 3C9B | CEO | PAO |
| A360 | 3C9C | CEO | PAO |
| A361 | 3A1A | CO | PAO |
| A362 | 3A1B | CO | PAO |
| A363 | 3A1C | CO | PAO |
| A364 | 3A2A | CO | PAO |
| A365 | 3A2B | CO | PAO |
| A366 | 3A2C | CO | PAO |
| A367 | 3A3A | CO | PAO |
| A368 | 3A3B | CO | PAO |
| A369 | 3A3C | CO | PAO |
| A370 | 3B1A | CO | PAO |
| A371 | 3B1B | CO | PAO |
| A372 | 3B1C | CO | PAO |
| A373 | 3B2A | CO | PAO |
| A374 | 3B2B | CO | PAO |
| A375 | 3B2C | CO | PAO |
| A376 | 3B3A | CO | PAO |
| A377 | 3B3B | CO | PAO |
| A378 | 3B3C | CO | PAO |
| A379 | 3C1A | CO | PAO |
| A380 | 3C1B | CO | PAO |
| A381 | 3C1C | CO | PAO |
| A382 | 3C2A | CO | PAO |
| A383 | 3C2B | CO | PAO |
| A384 | 3C2C | CO | PAO |
| A385 | 3C3A | CO | PAO |
| A386 | 3C3B | CO | PAO |
| A387 | 3C3C | CO | PAO |
| A388 | 3C4A | CO | PAO |
| A389 | 3C4B | CO | PAO |
| A390 | 3C4C | CO | PAO |
| A391 | 3C5A | CO | PAO |
| A392 | 3C5B | CO | PAO |
| A393 | 3C5C | CO | PAO |
| A394 | 3C6A | CO | PAO |
| A395 | 3C6B | CO | PAO |
| A396 | 3C6C | CO | PAO |
| A397 | 3C7A | CO | PAO |
| A398 | 3C7B | CO | PAO |
| A399 | 3C7C | CO | PAO |
| A400 | 3C8A | CO | PAO |
| A401 | 3C8B | CO | PAO |
| A402 | 3C8C | CO | PAO |
| A403 | 3C9A | CO | PAO |
| A404 | 3C9B | CO | PAO |
| A405 | 3C9C | CO | PAO |
| A406 | 4A1A | COMP | PAO |
| A407 | 4A1B | COMP | PAO |
| A408 | 4A1C | COMP | PAO |
| A409 | 4A2A | COMP | PAO |
| A410 | 4A2B | COMP | PAO |
| A411 | 4A2C | COMP | PAO |
| A412 | 4A3A | COMP | PAO |
| A413 | 4A3B | COMP | PAO |
| A414 | 4A3C | COMP | PAO |
| A415 | 4B1A | COMP | PAO |
| A416 | 4B1B | COMP | PAO |
| A417 | 4B1C | COMP | PAO |
| A418 | 4B2A | COMP | PAO |
| A419 | 4B2B | COMP | PAO |
| A420 | 4B2C | COMP | PAO |
| A421 | 4B3A | COMP | PAO |
| A422 | 4B3B | COMP | PAO |
| A423 | 4B3C | COMP | PAO |
| A424 | 4C1A | COMP | PAO |
| A425 | 4C1B | COMP | PAO |
| A426 | 4C1C | COMP | PAO |
| A427 | 4C2A | COMP | PAO |
| A428 | 4C2B | COMP | PAO |
| A429 | 4C2C | COMP | PAO |
| A430 | 4C3A | COMP | PAO |
| A431 | 4C3B | COMP | PAO |
| A432 | 4C3C | COMP | PAO |
| A433 | 4C4A | COMP | PAO |
| A434 | 4C4B | COMP | PAO |
| A435 | 4C4C | COMP | PAO |
| A436 | 4C5A | COMP | PAO |
| A437 | 4C5B | COMP | PAO |
| A438 | 4C5C | COMP | PAO |
| A439 | 4C6A | COMP | PAO |
| A440 | 4C6B | COMP | PAO |
| A441 | 4C6C | COMP | PAO |
| A442 | 4C7A | COMP | PAO |
| A443 | 4C7B | COMP | PAO |
| A444 | 4C7C | COMP | PAO |
| A445 | 4C8A | COMP | PAO |
| A446 | 4C8B | COMP | PAO |
| A447 | 4C8C | COMP | PAO |
| A448 | 4C9A | COMP | PAO |
| A449 | 4C9B | COMP | PAO |
| A450 | 4C9C | COMP | PAO |
| A451 | 4A1A | CEO | PAO |
| A452 | 4A1B | CEO | PAO |
| A453 | 4A1C | CEO | PAO |
| A454 | 4A2A | CEO | PAO |
| A455 | 4A2B | CEO | PAO |
| A456 | 4A2C | CEO | PAO |
| A457 | 4A3A | CEO | PAO |
| A458 | 4A3B | CEO | PAO |
| A459 | 4A3C | CEO | PAO |
| A460 | 4B1A | CEO | PAO |
| A461 | 4B1B | CEO | PAO |
| A462 | 4B1C | CEO | PAO |
| A463 | 4B2A | CEO | PAO |
| A464 | 4B2B | CEO | PAO |
| A465 | 4B2C | CEO | PAO |
| A466 | 4B3A | CEO | PAO |
| A467 | 4B3B | CEO | PAO |
| A468 | 4B3C | CEO | PAO |
| A469 | 4C1A | CEO | PAO |
| A470 | 4C1B | CEO | PAO |
| A471 | 4C1C | CEO | PAO |
| A472 | 4C2A | CEO | PAO |
| A473 | 4C2B | CEO | PAO |
| A474 | 4C2C | CEO | PAO |
| A475 | 4C3A | CEO | PAO |
| A476 | 4C3B | CEO | PAO |
| A477 | 4C3C | CEO | PAO |
| A478 | 4C4A | CEO | PAO |
| A479 | 4C4B | CEO | PAO |
| A480 | 4C4C | CEO | PAO |
| A481 | 4C5A | CEO | PAO |
| A482 | 4C5B | CEO | PAO |
| A483 | 4C5C | CEO | PAO |
| A484 | 4C6A | CEO | PAO |
| A485 | 4C6B | CEO | PAO |
| A486 | 4C6C | CEO | PAO |
| A487 | 4C7A | CEO | PAO |
| A488 | 4C7B | CEO | PAO |
| A489 | 4C7C | CEO | PAO |
| A490 | 4C8A | CEO | PAO |
| A491 | 4C8B | CEO | PAO |
| A492 | 4C8C | CEO | PAO |
| A493 | 4C9A | CEO | PAO |
| A494 | 4C9B | CEO | PAO |
| A495 | 4C9C | CEO | PAO |
| A496 | 4A1A | CO | PAO |
| A497 | 4A1B | CO | PAO |
| A498 | 4A1C | CO | PAO |
| A499 | 4A2A | CO | PAO |
| A500 | 4A2B | CO | PAO |
| A501 | 4A2C | CO | PAO |
| A502 | 4A3A | CO | PAO |
| A503 | 4A3B | CO | PAO |
| A504 | 4A3C | CO | PAO |
| A505 | 4B1A | CO | PAO |
| A506 | 4B1B | CO | PAO |
| A507 | 4B1C | CO | PAO |
| A508 | 4B2A | CO | PAO |
| A509 | 4B2B | CO | PAO |
| A510 | 4B2C | CO | PAO |
| A511 | 4B3A | CO | PAO |
| A512 | 4B3B | CO | PAO |
| A513 | 4B3C | CO | PAO |
| A514 | 4C1A | CO | PAO |
| A515 | 4C1B | CO | PAO |
| A516 | 4C1C | CO | PAO |
| A517 | 4C2A | CO | PAO |
| A518 | 4C2B | CO | PAO |
| A519 | 4C2C | CO | PAO |
| A520 | 4C3A | CO | PAO |
| A521 | 4C3B | CO | PAO |
| A522 | 4C3C | CO | PAO |
| A523 | 4C4A | CO | PAO |
| A524 | 4C4B | CO | PAO |
| A525 | 4C4C | CO | PAO |
| A526 | 4C5A | CO | PAO |
| A527 | 4C5B | CO | PAO |
| A528 | 4C5C | CO | PAO |
| A529 | 4C6A | CO | PAO |
| A530 | 4C6B | CO | PAO |
| A531 | 4C6C | CO | PAO |
| A532 | 4C7A | CO | PAO |
| A533 | 4C7B | CO | PAO |
| A534 | 4C7C | CO | PAO |
| A535 | 4C8A | CO | PAO |
| A536 | 4C8B | CO | PAO |
| A537 | 4C8C | CO | PAO |
| A538 | 4C9A | CO | PAO |
| A539 | 4C9B | CO | PAO |
| A540 | 4C9C | CO | PAO |

Polyol Ester (POE) Luibricant

| | | | |
|---|---|---|---|
| B1 | 1A1A | COMP | POE |
| B2 | 1A1B | COMP | POE |
| B3 | 1A1C | COMP | POE |
| B4 | 1A2A | COMP | POE |
| B5 | 1A2B | COMP | POE |
| B6 | 1A2C | COMP | POE |
| B7 | 1A3A | COMP | POE |
| B8 | 1A3B | COMP | POE |
| B9 | 1A3C | COMP | POE |
| B10 | 1B1A | COMP | POE |
| B11 | 1B1B | COMP | POE |
| B12 | 1B1C | COMP | POE |
| B13 | 1B2A | COMP | POE |
| B14 | 1B2B | COMP | POE |
| B15 | 1B2C | COMP | POE |
| B16 | 1B3A | COMP | POE |
| B17 | 1B3B | COMP | POE |
| B18 | 1B3C | COMP | POE |
| B19 | 1C1A | COMP | POE |
| B20 | 1C1B | COMP | POE |
| B21 | 1C1C | COMP | POE |
| B22 | 1C2A | COMP | POE |
| B23 | 1C2B | COMP | POE |
| B24 | 1C2C | COMP | POE |
| B25 | 1C3A | COMP | POE |
| B26 | 1C3B | COMP | POE |
| B27 | 1C3C | COMP | POE |
| B28 | 1C4A | COMP | POE |
| B29 | 1C4B | COMP | POE |
| B30 | 1C4C | COMP | POE |
| B31 | 1C5A | COMP | POE |
| B32 | 1C5B | COMP | POE |
| B33 | 1C5C | COMP | POE |
| B34 | 1C6A | COMP | POE |
| B35 | 1C6B | COMP | POE |
| B36 | 1C6C | COMP | POE |
| B37 | 1C7A | COMP | POE |
| B38 | 1C7B | COMP | POE |
| B39 | 1C7C | COMP | POE |
| B40 | 1C8A | COMP | POE |
| B41 | 1C8B | COMP | POE |
| B42 | 1C8C | COMP | POE |
| B43 | 1C9A | COMP | POE |
| B44 | 1C9B | COMP | POE |
| B45 | 1C9C | COMP | POE |
| B46 | 1A1A | CEO | POE |
| B47 | 1A1B | CEO | POE |
| B48 | 1A1C | CEO | POE |
| B49 | 1A2A | CEO | POE |
| B50 | 1A2B | CEO | POE |
| B51 | 1A2C | CEO | POE |
| B52 | 1A3A | CEO | POE |
| B53 | 1A3B | CEO | POE |
| B54 | 1A3C | CEO | POE |
| B55 | 1B1A | CEO | POE |
| B56 | 1B1B | CEO | POE |
| B57 | 1B1C | CEO | POE |
| B58 | 1B2A | CEO | POE |
| B59 | 1B2B | CEO | POE |
| B60 | 1B2C | CEO | POE |
| B61 | 1B3A | CEO | POE |
| B62 | 1B3B | CEO | POE |
| B63 | 1B3C | CEO | POE |
| B64 | 1C1A | CEO | POE |
| B65 | 1C1B | CEO | POE |
| B66 | 1C1C | CEO | POE |
| B67 | 1C2A | CEO | POE |
| B68 | 1C2B | CEO | POE |
| B69 | 1C2C | CEO | POE |
| B70 | 1C3A | CEO | POE |
| B71 | 1C3B | CEO | POE |
| B72 | 1C3C | CEO | POE |
| B73 | 1C4A | CEO | POE |
| B74 | 1C4B | CEO | POE |
| B75 | 1C4C | CEO | POE |
| B76 | 1C5A | CEO | POE |
| B77 | 1C5B | CEO | POE |
| B78 | 1C5C | CEO | POE |
| B79 | 1C6A | CEO | POE |
| B80 | 1C6B | CEO | POE |
| B81 | 1C6C | CEO | POE |
| B82 | 1C7A | CEO | POE |
| B83 | 1C7B | CEO | POE |
| B84 | 1C7C | CEO | POE |
| B85 | 1C8A | CEO | POE |
| B86 | 1C8B | CEO | POE |
| B87 | 1C8C | CEO | POE |
| B88 | 1C9A | CEO | POE |
| B89 | 1C9B | CEO | POE |
| B90 | 1C9C | CEO | POE |
| B91 | 1A1A | CO | POE |
| B92 | 1A1B | CO | POE |
| B93 | 1A1C | CO | POE |
| B94 | 1A2A | CO | POE |
| B95 | 1A2B | CO | POE |
| B96 | 1A2C | CO | POE |
| B97 | 1A3A | CO | POE |
| B98 | 1A3B | CO | POE |
| B99 | 1A3C | CO | POE |
| B100 | 1B1A | CO | POE |
| B101 | 1B1B | CO | POE |
| B102 | 1B1C | CO | POE |
| B103 | 1B2A | CO | POE |
| B104 | 1B2B | CO | POE |
| B105 | 1B2C | CO | POE |
| B106 | 1B3A | CO | POE |
| B107 | 1B3B | CO | POE |
| B108 | 1B3C | CO | POE |
| B109 | 1C1A | CO | POE |
| B110 | 1C1B | CO | POE |
| B111 | 1C1C | CO | POE |
| B112 | 1C2A | CO | POE |
| B113 | 1C2B | CO | POE |
| B114 | 1C2C | CO | POE |
| B115 | 1C3A | CO | POE |
| B116 | 1C3B | CO | POE |
| B117 | 1C3C | CO | POE |
| B118 | 1C4A | CO | POE |
| B119 | 1C4B | CO | POE |
| B120 | 1C4C | CO | POE |
| B121 | 1C5A | CO | POE |
| B122 | 1C5B | CO | POE |
| B123 | 1C5C | CO | POE |
| B124 | 1C6A | CO | POE |
| B125 | 1C6B | CO | POE |
| B126 | 1C6C | CO | POE |
| B127 | 1C7A | CO | POE |
| B128 | 1C7B | CO | POE |
| B129 | 1C7C | CO | POE |
| B130 | 1C8A | CO | POE |
| B131 | 1C8B | CO | POE |
| B132 | 1C8C | CO | POE |
| B133 | 1C9A | CO | POE |
| B134 | 1C9B | CO | POE |
| B135 | 1C9C | CO | POE |
| B136 | 2A1A | COMP | POE |
| B137 | 2A1B | COMP | POE |
| B138 | 2A1C | COMP | POE |
| B139 | 2A2A | COMP | POE |
| B140 | 2A2B | COMP | POE |
| B141 | 2A2C | COMP | POE |
| B142 | 2A3A | COMP | POE |
| B143 | 2A3B | COMP | POE |
| B144 | 2A3C | COMP | POE |
| B145 | 2B1A | COMP | POE |
| B146 | 2B1B | COMP | POE |
| B147 | 2B1C | COMP | POE |
| B148 | 2B2A | COMP | POE |
| B149 | 2B2B | COMP | POE |
| B150 | 2B2C | COMP | POE |
| B151 | 2B3A | COMP | POE |
| B152 | 2B3B | COMP | POE |
| B153 | 2B3C | COMP | POE |
| B154 | 2C1A | COMP | POE |
| B155 | 2C1B | COMP | POE |
| B156 | 2C1C | COMP | POE |
| B157 | 2C2A | COMP | POE |
| B158 | 2C2B | COMP | POE |
| B159 | 2C2C | COMP | POE |
| B160 | 2C3A | COMP | POE |
| B161 | 2C3B | COMP | POE |
| B162 | 2C3C | COMP | POE |
| B163 | 2C4A | COMP | POE |
| B164 | 2C4B | COMP | POE |
| B165 | 2C4C | COMP | POE |
| B166 | 2C5A | COMP | POE |
| B167 | 2C5B | COMP | POE |
| B168 | 2C5C | COMP | POE |
| B169 | 2C6A | COMP | POE |
| B170 | 2C6B | COMP | POE |
| B171 | 2C6C | COMP | POE |
| B172 | 2C7A | COMP | POE |
| B173 | 2C7B | COMP | POE |
| B174 | 2C7C | COMP | POE |
| B175 | 2C8A | COMP | POE |
| B176 | 2C8B | COMP | POE |
| B177 | 2C8C | COMP | POE |
| B178 | 2C9A | COMP | POE |
| B179 | 2C9B | COMP | POE |
| B180 | 2C9C | COMP | POE |
| B181 | 2A1A | CEO | POE |
| B182 | 2A1B | CEO | POE |
| B183 | 2A1C | CEO | POE |
| B184 | 2A2A | CEO | POE |
| B185 | 2A2B | CEO | POE |
| B186 | 2A2C | CEO | POE |
| B187 | 2A3A | CEO | POE |
| B188 | 2A3B | CEO | POE |
| B189 | 2A3C | CEO | POE |
| B190 | 2B1A | CEO | POE |
| B191 | 2B1B | CEO | POE |
| B192 | 2B1C | CEO | POE |
| B193 | 2B2A | CEO | POE |
| B194 | 2B2B | CEO | POE |
| B195 | 2B2C | CEO | POE |
| B196 | 2B3A | CEO | POE |
| B197 | 2B3B | CEO | POE |
| B198 | 2B3C | CEO | POE |
| B199 | 2C1A | CEO | POE |
| B200 | 2C1B | CEO | POE |
| B201 | 2C1C | CEO | POE |
| B202 | 2C2A | CEO | POE |
| B203 | 2C2B | CEO | POE |
| B204 | 2C2C | CEO | POE |
| B205 | 2C3A | CEO | POE |
| B206 | 2C3B | CEO | POE |
| B207 | 2C3C | CEO | POE |
| B208 | 2C4A | CEO | POE |
| B209 | 2C4B | CEO | POE |
| B210 | 2C4C | CEO | POE |
| B211 | 2C5A | CEO | POE |
| B212 | 2C5B | CEO | POE |
| B213 | 2C5C | CEO | POE |
| B214 | 2C6A | CEO | POE |
| B215 | 2C6B | CEO | POE |
| B216 | 2C6C | CEO | POE |
| B217 | 2C7A | CEO | POE |
| B218 | 2C7B | CEO | POE |
| B219 | 2C7C | CEO | POE |
| B220 | 2C8A | CEO | POE |
| B221 | 2C8B | CEO | POE |
| B222 | 2C8C | CEO | POE |
| B223 | 2C9A | CEO | POE |
| B224 | 2C9B | CEO | POE |
| B225 | 2C9C | CEO | POE |
| B226 | 2A1A | CO | POE |
| B227 | 2A1B | CO | POE |
| B228 | 2A1C | CO | POE |
| B229 | 2A2A | CO | POE |
| B230 | 2A2B | CO | POE |
| B231 | 2A2C | CO | POE |
| B232 | 2A3A | CO | POE |
| B233 | 2A3B | CO | POE |
| B234 | 2A3C | CO | POE |
| B235 | 2B1A | CO | POE |
| B236 | 2B1B | CO | POE |
| B237 | 2B1C | CO | POE |
| B238 | 2B2A | CO | POE |
| B239 | 2B2B | CO | POE |
| B240 | 2B2C | CO | POE |
| B241 | 2B3A | CO | POE |
| B242 | 2B3B | CO | POE |
| B243 | 2B3C | CO | POE |
| B244 | 2C1A | CO | POE |
| B245 | 2C1B | CO | POE |
| B246 | 2C1C | CO | POE |
| B247 | 2C2A | CO | POE |
| B248 | 2C2B | CO | POE |
| B249 | 2C2C | CO | POE |
| B250 | 2C3A | CO | POE |
| B251 | 2C3B | CO | POE |
| B252 | 2C3C | CO | POE |
| B253 | 2C4A | CO | POE |
| B254 | 2C4B | CO | POE |
| B255 | 2C4C | CO | POE |
| B256 | 2C5A | CO | POE |
| B257 | 2C5B | CO | POE |
| B258 | 2C5C | CO | POE |
| B259 | 2C6A | CO | POE |
| B260 | 2C6B | CO | POE |
| B261 | 2C6C | CO | POE |
| B262 | 2C7A | CO | POE |
| B263 | 2C7B | CO | POE |
| B264 | 2C7C | CO | POE |
| B265 | 2C8A | CO | POE |
| B266 | 2C8B | CO | POE |
| B267 | 2C8C | CO | POE |
| B268 | 2C9A | CO | POE |
| B269 | 2C9B | CO | POE |
| B270 | 2C9C | CO | POE |
| B271 | 3A1A | COMP | POE |
| B272 | 3A1B | COMP | POE |
| B273 | 3A1C | COMP | POE |
| B274 | 3A2A | COMP | POE |
| B275 | 3A2B | COMP | POE |
| B276 | 3A2C | COMP | POE |
| B277 | 3A3A | COMP | POE |
| B278 | 3A3B | COMP | POE |
| B279 | 3A3C | COMP | POE |
| B280 | 3B1A | COMP | POE |
| B281 | 3B1B | COMP | POE |
| B282 | 3B1C | COMP | POE |
| B283 | 3B2A | COMP | POE |
| B284 | 3B2B | COMP | POE |
| B285 | 3B2C | COMP | POE |
| B286 | 3B3A | COMP | POE |
| B287 | 3B3B | COMP | POE |
| B288 | 3B3C | COMP | POE |
| B289 | 3C1A | COMP | POE |
| B290 | 3C1B | COMP | POE |
| B291 | 3C1C | COMP | POE |
| B292 | 3C2A | COMP | POE |
| B293 | 3C2B | COMP | POE |
| B294 | 3C2C | COMP | POE |
| B295 | 3C3A | COMP | POE |
| B296 | 3C3B | COMP | POE |
| B297 | 3C3C | COMP | POE |
| B298 | 3C4A | COMP | POE |
| B299 | 3C4B | COMP | POE |
| B300 | 3C4C | COMP | POE |
| B301 | 3C5A | COMP | POE |
| B302 | 3C5B | COMP | POE |
| B303 | 3C5C | COMP | POE |
| B304 | 3C6A | COMP | POE |
| B305 | 3C6B | COMP | POE |
| B306 | 3C6C | COMP | POE |
| B307 | 3C7A | COMP | POE |
| B308 | 3C7B | COMP | POE |
| B309 | 3C7C | COMP | POE |
| B310 | 3C8A | COMP | POE |
| B311 | 3C8B | COMP | POE |
| B312 | 3C8C | COMP | POE |
| B313 | 3C9A | COMP | POE |
| B314 | 3C9B | COMP | POE |
| B315 | 3C9C | COMP | POE |
| B316 | 3A1A | CEO | POE |
| B317 | 3A1B | CEO | POE |
| B318 | 3A1C | CEO | POE |
| B319 | 3A2A | CEO | POE |
| B320 | 3A2B | CEO | POE |
| B321 | 3A2C | CEO | POE |
| B322 | 3A3A | CEO | POE |
| B323 | 3A3B | CEO | POE |
| B324 | 3A3C | CEO | POE |
| B325 | 3B1A | CEO | POE |
| B326 | 3B1B | CEO | POE |
| B327 | 3B1C | CEO | POE |
| B328 | 3B2A | CEO | POE |
| B329 | 3B2B | CEO | POE |
| B330 | 3B2C | CEO | POE |
| B331 | 3B3A | CEO | POE |
| B332 | 3B3B | CEO | POE |
| B333 | 3B3C | CEO | POE |
| B334 | 3C1A | CEO | POE |
| B335 | 3C1B | CEO | POE |
| B336 | 3C1C | CEO | POE |
| B337 | 3C2A | CEO | POE |
| B338 | 3C2B | CEO | POE |
| B339 | 3C2C | CEO | POE |
| B340 | 3C3A | CEO | POE |
| B341 | 3C3B | CEO | POE |
| B342 | 3C3C | CEO | POE |
| B343 | 3C4A | CEO | POE |
| B344 | 3C4B | CEO | POE |
| B345 | 3C4C | CEO | POE |
| B346 | 3C5A | CEO | POE |
| B347 | 3C5B | CEO | POE |
| B348 | 3C5C | CEO | POE |
| B349 | 3C6A | CEO | POE |
| B350 | 3C6B | CEO | POE |
| B351 | 3C6C | CEO | POE |
| B352 | 3C7A | CEO | POE |
| B353 | 3C7B | CEO | POE |
| B354 | 3C7C | CEO | POE |
| B355 | 3C8A | CEO | POE |
| B356 | 3C8B | CEO | POE |
| B357 | 3C8C | CEO | POE |
| B358 | 3C9A | CEO | POE |
| B359 | 3C9B | CEO | POE |
| B360 | 3C9C | CEO | POE |
| B361 | 3A1A | CO | POE |
| B362 | 3A1B | CO | POE |
| B363 | 3A1C | CO | POE |
| B364 | 3A2A | CO | POE |
| B365 | 3A2B | CO | POE |
| B366 | 3A2C | CO | POE |
| B367 | 3A3A | CO | POE |
| B368 | 3A3B | CO | POE |
| B369 | 3A3C | CO | POE |
| B370 | 3B1A | CO | POE |
| B371 | 3B1B | CO | POE |
| B372 | 3B1C | CO | POE |
| B373 | 3B2A | CO | POE |
| B374 | 3B2B | CO | POE |
| B375 | 3B2C | CO | POE |
| B376 | 3B3A | CO | POE |
| B377 | 3B3B | CO | POE |
| B378 | 3B3C | CO | POE |
| B379 | 3C1A | CO | POE |
| B380 | 3C1B | CO | POE |
| B381 | 3C1C | CO | POE |
| B382 | 3C2A | CO | POE |
| B383 | 3C2B | CO | POE |
| B384 | 3C2C | CO | POE |
| B385 | 3C3A | CO | POE |
| B386 | 3C3B | CO | POE |
| B387 | 3C3C | CO | POE |
| B388 | 3C4A | CO | POE |
| B389 | 3C4B | CO | POE |
| B390 | 3C4C | CO | POE |
| B391 | 3C5A | CO | POE |
| B392 | 3C5B | CO | POE |
| B393 | 3C5C | CO | POE |
| B394 | 3C6A | CO | POE |
| B395 | 3C6B | CO | POE |
| B396 | 3C6C | CO | POE |
| B397 | 3C7A | CO | POE |
| B398 | 3C7B | CO | POE |
| B399 | 3C7C | CO | POE |
| B400 | 3C8A | CO | POE |
| B401 | 3C8B | CO | POE |
| B402 | 3C8C | CO | POE |
| B403 | 3C9A | CO | POE |
| B404 | 3C9B | CO | POE |
| B405 | 3C9C | CO | POE |
| B406 | 4A1A | COMP | POE |
| B407 | 4A1B | COMP | POE |
| B408 | 4A1C | COMP | POE |
| B409 | 4A2A | COMP | POE |
| B410 | 4A2B | COMP | POE |
| B411 | 4A2C | COMP | POE |
| B412 | 4A3A | COMP | POE |
| B413 | 4A3B | COMP | POE |
| B414 | 4A3C | COMP | POE |
| B415 | 4B1A | COMP | POE |
| B416 | 4B1B | COMP | POE |
| B417 | 4B1C | COMP | POE |
| B418 | 4B2A | COMP | POE |
| B419 | 4B2B | COMP | POE |
| B420 | 4B2C | COMP | POE |
| B421 | 4B3A | COMP | POE |
| B422 | 4B3B | COMP | POE |
| B423 | 4B3C | COMP | POE |
| B424 | 4C1A | COMP | POE |
| B425 | 4C1B | COMP | POE |
| B426 | 4C1C | COMP | POE |
| B427 | 4C2A | COMP | POE |
| B428 | 4C2B | COMP | POE |
| B429 | 4C2C | COMP | POE |
| B430 | 4C3A | COMP | POE |
| B431 | 4C3B | COMP | POE |
| B432 | 4C3C | COMP | POE |
| B433 | 4C4A | COMP | POE |
| B434 | 4C4B | COMP | POE |
| B435 | 4C4C | COMP | POE |
| B436 | 4C5A | COMP | POE |
| B437 | 4C5B | COMP | POE |
| B438 | 4C5C | COMP | POE |
| B439 | 4C6A | COMP | POE |
| B440 | 4C6B | COMP | POE |
| B441 | 4C6C | COMP | POE |
| B442 | 4C7A | COMP | POE |
| B443 | 4C7B | COMP | POE |
| B444 | 4C7C | COMP | POE |
| B445 | 4C8A | COMP | POE |
| B446 | 4C8B | COMP | POE |
| B447 | 4C8C | COMP | POE |
| B448 | 4C9A | COMP | POE |
| B449 | 4C9B | COMP | POE |
| B450 | 4C9C | COMP | POE |
| B451 | 4A1A | CEO | POE |
| B452 | 4A1B | CEO | POE |
| B453 | 4A1C | CEO | POE |
| B454 | 4A2A | CEO | POE |
| B455 | 4A2B | CEO | POE |
| B456 | 4A2C | CEO | POE |
| B457 | 4A3A | CEO | POE |
| B458 | 4A3B | CEO | POE |
| B459 | 4A3C | CEO | POE |
| B460 | 4B1A | CEO | POE |
| B461 | 4B1B | CEO | POE |
| B462 | 4B1C | CEO | POE |
| B463 | 4B2A | CEO | POE |
| B464 | 4B2B | CEO | POE |
| B465 | 4B2C | CEO | POE |
| B466 | 4B3A | CEO | POE |
| B467 | 4B3B | CEO | POE |
| B468 | 4B3C | CEO | POE |
| B469 | 4C1A | CEO | POE |
| B470 | 4C1B | CEO | POE |
| B471 | 4C1C | CEO | POE |
| B472 | 4C2A | CEO | POE |
| B473 | 4C2B | CEO | POE |
| B474 | 4C2C | CEO | POE |
| B475 | 4C3A | CEO | POE |
| B476 | 4C3B | CEO | POE |
| B477 | 4C3C | CEO | POE |
| B478 | 4C4A | CEO | POE |
| B479 | 4C4B | CEO | POE |
| B480 | 4C4C | CEO | POE |
| B481 | 4C5A | CEO | POE |
| B482 | 4C5B | CEO | POE |
| B483 | 4C5C | CEO | POE |
| B484 | 4C6A | CEO | POE |
| B485 | 4C6B | CEO | POE |
| B486 | 4C6C | CEO | POE |
| B487 | 4C7A | CEO | POE |
| B488 | 4C7B | CEO | POE |
| B489 | 4C7C | CEO | POE |
| B490 | 4C8A | CEO | POE |
| B491 | 4C8B | CEO | POE |
| B492 | 4C8C | CEO | POE |
| B493 | 4C9A | CEO | POE |
| B494 | 4C9B | CEO | POE |
| B495 | 4C9C | CEO | POE |
| B496 | 4A1A | CO | POE |
| B497 | 4A1B | CO | POE |
| B498 | 4A1C | CO | POE |
| B499 | 4A2A | CO | POE |
| B500 | 4A2B | CO | POE |
| B501 | 4A2C | CO | POE |
| B502 | 4A3A | CO | POE |
| B503 | 4A3B | CO | POE |
| B504 | 4A3C | CO | POE |
| B505 | 4B1A | CO | POE |
| B506 | 4B1B | CO | POE |
| B507 | 4B1C | CO | POE |
| B508 | 4B2A | CO | POE |
| B509 | 4B2B | CO | POE |
| B510 | 4B2C | CO | POE |
| B511 | 4B3A | CO | POE |
| B512 | 4B3B | CO | POE |
| B513 | 4B3C | CO | POE |
| B514 | 4C1A | CO | POE |
| B515 | 4C1B | CO | POE |
| B516 | 4C1C | CO | POE |
| B517 | 4C2A | CO | POE |
| B518 | 4C2B | CO | POE |
| B519 | 4C2C | CO | POE |
| B520 | 4C3A | CO | POE |
| B521 | 4C3B | CO | POE |
| B522 | 4C3C | CO | POE |
| B523 | 4C4A | CO | POE |
| B524 | 4C4B | CO | POE |
| B525 | 4C4C | CO | POE |
| B526 | 4C5A | CO | POE |
| B527 | 4C5B | CO | POE |
| B528 | 4C5C | CO | POE |
| B529 | 4C6A | CO | POE |
| B530 | 4C6B | CO | POE |
| B531 | 4C6C | CO | POE |
| B532 | 4C7A | CO | POE |
| B533 | 4C7B | CO | POE |
| B534 | 4C7C | CO | POE |
| B535 | 4C8A | CO | POE |
| B536 | 4C8B | CO | POE |
| B537 | 4C8C | CO | POE |
| B538 | 4C9A | CO | POE |
| B539 | 4C9B | CO | POE |
| B540 | 4C9C | CO | POE |

Mineral Oil Luibricant

| | | | |
|---|---|---|---|
| C1 | 1A1A | COMP | Mineral |
| C2 | 1A1B | COMP | Mineral |
| C3 | 1A1C | COMP | Mineral |
| C4 | 1A2A | COMP | Mineral |
| C5 | 1A2B | COMP | Mineral |
| C6 | 1A2C | COMP | Mineral |
| C7 | 1A3A | COMP | Mineral |
| C8 | 1A3B | COMP | Mineral |
| C9 | 1A3C | COMP | Mineral |
| C10 | 1B1A | COMP | Mineral |
| C11 | 1B1B | COMP | Mineral |
| C12 | 1B1C | COMP | Mineral |
| C13 | 1B2A | COMP | Mineral |
| C14 | 1B2B | COMP | Mineral |
| C15 | 1B2C | COMP | Mineral |
| C16 | 1B3A | COMP | Mineral |
| C17 | 1B3B | COMP | Mineral |
| C18 | 1B3C | COMP | Mineral |
| C19 | 1C1A | COMP | Mineral |
| C20 | 1C1B | COMP | Mineral |
| C21 | 1C1C | COMP | Mineral |
| C22 | 1C2A | COMP | Mineral |
| C23 | 1C2B | COMP | Mineral |
| C24 | 1C2C | COMP | Mineral |
| C25 | 1C3A | COMP | Mineral |
| C26 | 1C3B | COMP | Mineral |
| C27 | 1C3C | COMP | Mineral |
| C28 | 1C4A | COMP | Mineral |
| C29 | 1C4B | COMP | Mineral |
| C30 | 1C4C | COMP | Mineral |
| C31 | 1C5A | COMP | Mineral |
| C32 | 1C5B | COMP | Mineral |
| C33 | 1C5C | COMP | Mineral |
| C34 | 1C6A | COMP | Mineral |
| C35 | 1C6B | COMP | Mineral |
| C36 | 1C6C | COMP | Mineral |
| C37 | 1C7A | COMP | Mineral |
| C38 | 1C7B | COMP | Mineral |
| C39 | 1C7C | COMP | Mineral |
| C40 | 1C8A | COMP | Mineral |
| C41 | 1C8B | COMP | Mineral |
| C42 | 1C8C | COMP | Mineral |
| C43 | 1C9A | COMP | Mineral |
| C44 | 1C9B | COMP | Mineral |
| C45 | 1C9C | COMP | Mineral |
| C46 | 1A1A | CEO | Mineral |
| C47 | 1A1B | CEO | Mineral |
| C48 | 1A1C | CEO | Mineral |
| C49 | 1A2A | CEO | Mineral |
| C50 | 1A2B | CEO | Mineral |
| C51 | 1A2C | CEO | Mineral |
| C52 | 1A3A | CEO | Mineral |
| C53 | 1A3B | CEO | Mineral |
| C54 | 1A3C | CEO | Mineral |
| C55 | 1B1A | CEO | Mineral |
| C56 | 1B1B | CEO | Mineral |
| C57 | 1B1C | CEO | Mineral |
| C58 | 1B2A | CEO | Mineral |
| C59 | 1B2B | CEO | Mineral |
| C60 | 1B2C | CEO | Mineral |
| C61 | 1B3A | CEO | Mineral |
| C62 | 1B3B | CEO | Mineral |
| C63 | 1B3C | CEO | Mineral |
| C64 | 1C1A | CEO | Mineral |
| C65 | 1C1B | CEO | Mineral |
| C66 | 1C1C | CEO | Mineral |
| C67 | 1C2A | CEO | Mineral |
| C68 | 1C2B | CEO | Mineral |
| C69 | 1C2C | CEO | Mineral |
| C70 | 1C3A | CEO | Mineral |
| C71 | 1C3B | CEO | Mineral |
| C72 | 1C3C | CEO | Mineral |
| C73 | 1C4A | CEO | Mineral |
| C74 | 1C4B | CEO | Mineral |
| C75 | 1C4C | CEO | Mineral |
| C76 | 1C5A | CEO | Mineral |
| C77 | 1C5B | CEO | Mineral |
| C78 | 1C5C | CEO | Mineral |
| C79 | 1C6A | CEO | Mineral |
| C80 | 1C6B | CEO | Mineral |
| C81 | 1C6C | CEO | Mineral |
| C82 | 1C7A | CEO | Mineral |
| C83 | 1C7B | CEO | Mineral |
| C84 | 1C7C | CEO | Mineral |
| C85 | 1C8A | CEO | Mineral |
| C86 | 1C8B | CEO | Mineral |
| C87 | 1C8C | CEO | Mineral |
| C88 | 1C9A | CEO | Mineral |
| C89 | 1C9B | CEO | Mineral |
| C90 | 1C9C | CEO | Mineral |
| C91 | 1A1A | CO | Mineral |
| C92 | 1A1B | CO | Mineral |
| C93 | 1A1C | CO | Mineral |
| C94 | 1A2A | CO | Mineral |
| C95 | 1A2B | CO | Mineral |
| C96 | 1A2C | CO | Mineral |
| C97 | 1A3A | CO | Mineral |
| C98 | 1A3B | CO | Mineral |
| C99 | 1A3C | CO | Mineral |
| C100 | 1B1A | CO | Mineral |
| C101 | 1B1B | CO | Mineral |
| C102 | 1B1C | CO | Mineral |
| C103 | 1B2A | CO | Mineral |
| C104 | 1B2B | CO | Mineral |
| C105 | 1B2C | CO | Mineral |
| C106 | 1B3A | CO | Mineral |
| C107 | 1B3B | CO | Mineral |
| C108 | 1B3C | CO | Mineral |
| C109 | 1C1A | CO | Mineral |
| C110 | 1C1B | CO | Mineral |
| C111 | 1C1C | CO | Mineral |
| C112 | 1C2A | CO | Mineral |
| C113 | 1C2B | CO | Mineral |
| C114 | 1C2C | CO | Mineral |
| C115 | 1C3A | CO | Mineral |
| C116 | 1C3B | CO | Mineral |
| C117 | 1C3C | CO | Mineral |
| C118 | 1C4A | CO | Mineral |
| C119 | 1C4B | CO | Mineral |
| C120 | 1C4C | CO | Mineral |
| C121 | 1C5A | CO | Mineral |
| C122 | 1C5B | CO | Mineral |
| C123 | 1C5C | CO | Mineral |
| C124 | 1C6A | CO | Mineral |
| C125 | 1C6B | CO | Mineral |
| C126 | 1C6C | CO | Mineral |
| C127 | 1C7A | CO | Mineral |
| C128 | 1C7B | CO | Mineral |
| C129 | 1C7C | CO | Mineral |
| C130 | 1C8A | CO | Mineral |
| C131 | 1C8B | CO | Mineral |
| C132 | 1C8C | CO | Mineral |
| C133 | 1C9A | CO | Mineral |
| C134 | 1C9B | CO | Mineral |
| C135 | 1C9C | CO | Mineral |
| C136 | 2A1A | COMP | Mineral |
| C137 | 2A1B | COMP | Mineral |
| C138 | 2A1C | COMP | Mineral |
| C139 | 2A2A | COMP | Mineral |
| C140 | 2A2B | COMP | Mineral |
| C141 | 2A2C | COMP | Mineral |
| C142 | 2A3A | COMP | Mineral |
| C143 | 2A3B | COMP | Mineral |
| C144 | 2A3C | COMP | Mineral |
| C145 | 2B1A | COMP | Mineral |
| C146 | 2B1B | COMP | Mineral |
| C147 | 2B1C | COMP | Mineral |
| C148 | 2B2A | COMP | Mineral |
| C149 | 2B2B | COMP | Mineral |
| C150 | 2B2C | COMP | Mineral |
| C151 | 2B3A | COMP | Mineral |
| C152 | 2B3B | COMP | Mineral |
| C153 | 2B3C | COMP | Mineral |
| C154 | 2C1A | COMP | Mineral |
| C155 | 2C1B | COMP | Mineral |
| C156 | 2C1C | COMP | Mineral |
| C157 | 2C2A | COMP | Mineral |
| C158 | 2C2B | COMP | Mineral |
| C159 | 2C2C | COMP | Mineral |
| C160 | 2C3A | COMP | Mineral |
| C161 | 2C3B | COMP | Mineral |
| C162 | 2C3C | COMP | Mineral |
| C163 | 2C4A | COMP | Mineral |
| C164 | 2C4B | COMP | Mineral |
| C165 | 2C4C | COMP | Mineral |
| C166 | 2C5A | COMP | Mineral |
| C167 | 2C5B | COMP | Mineral |
| C168 | 2C5C | COMP | Mineral |
| C169 | 2C6A | COMP | Mineral |
| C170 | 2C6B | COMP | Mineral |
| C171 | 2C6C | COMP | Mineral |
| C172 | 2C7A | COMP | Mineral |
| C173 | 2C7B | COMP | Mineral |
| C174 | 2C7C | COMP | Mineral |
| C175 | 2C8A | COMP | Mineral |
| C176 | 2C8B | COMP | Mineral |
| C177 | 2C8C | COMP | Mineral |
| C178 | 2C9A | COMP | Mineral |
| C179 | 2C9B | COMP | Mineral |
| C180 | 2C9C | COMP | Mineral |
| C181 | 2A1A | CEO | Mineral |
| C182 | 2A1B | CEO | Mineral |
| C183 | 2A1C | CEO | Mineral |
| C184 | 2A2A | CEO | Mineral |
| C185 | 2A2B | CEO | Mineral |
| C186 | 2A2C | CEO | Mineral |
| C187 | 2A3A | CEO | Mineral |
| C188 | 2A3B | CEO | Mineral |
| C189 | 2A3C | CEO | Mineral |
| C190 | 2B1A | CEO | Mineral |
| C191 | 2B1B | CEO | Mineral |
| C192 | 2B1C | CEO | Mineral |
| C193 | 2B2A | CEO | Mineral |
| C194 | 2B2B | CEO | Mineral |
| C195 | 2B2C | CEO | Mineral |
| C196 | 2B3A | CEO | Mineral |
| C197 | 2B3B | CEO | Mineral |
| C198 | 2B3C | CEO | Mineral |
| C199 | 2C1A | CEO | Mineral |
| C200 | 2C1B | CEO | Mineral |
| C201 | 2C1C | CEO | Mineral |
| C202 | 2C2A | CEO | Mineral |
| C203 | 2C2B | CEO | Mineral |
| C204 | 2C2C | CEO | Mineral |
| C205 | 2C3A | CEO | Mineral |
| C206 | 2C3B | CEO | Mineral |
| C207 | 2C3C | CEO | Mineral |
| C208 | 2C4A | CEO | Mineral |
| C209 | 2C4B | CEO | Mineral |
| C210 | 2C4C | CEO | Mineral |
| C211 | 2C5A | CEO | Mineral |
| C212 | 2C5B | CEO | Mineral |
| C213 | 2C5C | CEO | Mineral |
| C214 | 2C6A | CEO | Mineral |
| C215 | 2C6B | CEO | Mineral |
| C216 | 2C6C | CEO | Mineral |
| C217 | 2C7A | CEO | Mineral |
| C218 | 2C7B | CEO | Mineral |
| C219 | 2C7C | CEO | Mineral |
| C220 | 2C8A | CEO | Mineral |
| C221 | 2C8B | CEO | Mineral |
| C222 | 2C8C | CEO | Mineral |
| C223 | 2C9A | CEO | Mineral |
| C224 | 2C9B | CEO | Mineral |
| C225 | 2C9C | CEO | Mineral |
| C226 | 2A1A | CO | Mineral |
| C227 | 2A1B | CO | Mineral |
| C228 | 2A1C | CO | Mineral |
| C229 | 2A2A | CO | Mineral |
| C230 | 2A2B | CO | Mineral |
| C231 | 2A2C | CO | Mineral |
| C232 | 2A3A | CO | Mineral |
| C233 | 2A3B | CO | Mineral |
| C234 | 2A3C | CO | Mineral |
| C235 | 2B1A | CO | Mineral |
| C236 | 2B1B | CO | Mineral |
| C237 | 2B1C | CO | Mineral |
| C238 | 2B2A | CO | Mineral |
| C239 | 2B2B | CO | Mineral |
| C240 | 2B2C | CO | Mineral |
| C241 | 2B3A | CO | Mineral |
| C242 | 2B3B | CO | Mineral |
| C243 | 2B3C | CO | Mineral |
| C244 | 2C1A | CO | Mineral |
| C245 | 2C1B | CO | Mineral |
| C246 | 2C1C | CO | Mineral |
| C247 | 2C2A | CO | Mineral |
| C248 | 2C2B | CO | Mineral |
| C249 | 2C2C | CO | Mineral |
| C250 | 2C3A | CO | Mineral |
| C251 | 2C3B | CO | Mineral |
| C252 | 2C3C | CO | Mineral |
| C253 | 2C4A | CO | Mineral |
| C254 | 2C4B | CO | Mineral |
| C255 | 2C4C | CO | Mineral |
| C256 | 2C5A | CO | Mineral |
| C257 | 2C5B | CO | Mineral |
| C258 | 2C5C | CO | Mineral |
| C259 | 2C6A | CO | Mineral |
| C260 | 2C6B | CO | Mineral |
| C261 | 2C6C | CO | Mineral |
| C262 | 2C7A | CO | Mineral |
| C263 | 2C7B | CO | Mineral |
| C264 | 2C7C | CO | Mineral |
| C265 | 2C8A | CO | Mineral |
| C266 | 2C8B | CO | Mineral |
| C267 | 2C8C | CO | Mineral |
| C268 | 2C9A | CO | Mineral |
| C269 | 2C9B | CO | Mineral |
| C270 | 2C9C | CO | Mineral |
| C271 | 3A1A | COMP | Mineral |
| C272 | 3A1B | COMP | Mineral |
| C273 | 3A1C | COMP | Mineral |
| C274 | 3A2A | COMP | Mineral |
| C275 | 3A2B | COMP | Mineral |
| C276 | 3A2C | COMP | Mineral |
| C277 | 3A3A | COMP | Mineral |
| C278 | 3A3B | COMP | Mineral |
| C279 | 3A3C | COMP | Mineral |
| C280 | 3B1A | COMP | Mineral |
| C281 | 3B1B | COMP | Mineral |
| C282 | 3B1C | COMP | Mineral |
| C283 | 3B2A | COMP | Mineral |
| C284 | 3B2B | COMP | Mineral |
| C285 | 3B2C | COMP | Mineral |
| C286 | 3B3A | COMP | Mineral |
| C287 | 3B3B | COMP | Mineral |
| C288 | 3B3C | COMP | Mineral |
| C289 | 3C1A | COMP | Mineral |
| C290 | 3C1B | COMP | Mineral |
| C291 | 3C1C | COMP | Mineral |
| C292 | 3C2A | COMP | Mineral |
| C293 | 3C2B | COMP | Mineral |
| C294 | 3C2C | COMP | Mineral |
| C295 | 3C3A | COMP | Mineral |
| C296 | 3C3B | COMP | Mineral |
| C297 | 3C3C | COMP | Mineral |
| C298 | 3C4A | COMP | Mineral |
| C299 | 3C4B | COMP | Mineral |
| C300 | 3C4C | COMP | Mineral |
| C301 | 3C5A | COMP | Mineral |
| C302 | 3C5B | COMP | Mineral |
| C303 | 3C5C | COMP | Mineral |
| C304 | 3C6A | COMP | Mineral |
| C305 | 3C6B | COMP | Mineral |
| C306 | 3C6C | COMP | Mineral |
| C307 | 3C7A | COMP | Mineral |
| C308 | 3C7B | COMP | Mineral |
| C309 | 3C7C | COMP | Mineral |
| C310 | 3C8A | COMP | Mineral |
| C311 | 3C8B | COMP | Mineral |
| C312 | 3C8C | COMP | Mineral |
| C313 | 3C9A | COMP | Mineral |
| C314 | 3C9B | COMP | Mineral |
| C315 | 3C9C | COMP | Mineral |
| C316 | 3A1A | CEO | Mineral |
| C317 | 3A1B | CEO | Mineral |
| C318 | 3A1C | CEO | Mineral |
| C319 | 3A2A | CEO | Mineral |
| C320 | 3A2B | CEO | Mineral |
| C321 | 3A2C | CEO | Mineral |
| C322 | 3A3A | CEO | Mineral |
| C323 | 3A3B | CEO | Mineral |
| C324 | 3A3C | CEO | Mineral |
| C325 | 3B1A | CEO | Mineral |
| C326 | 3B1B | CEO | Mineral |
| C327 | 3B1C | CEO | Mineral |
| C328 | 3B2A | CEO | Mineral |
| C329 | 3B2B | CEO | Mineral |
| C330 | 3B2C | CEO | Mineral |
| C331 | 3B3A | CEO | Mineral |
| C332 | 3B3B | CEO | Mineral |
| C333 | 3B3C | CEO | Mineral |
| C334 | 3C1A | CEO | Mineral |
| C335 | 3C1B | CEO | Mineral |
| C336 | 3C1C | CEO | Mineral |
| C337 | 3C2A | CEO | Mineral |
| C338 | 3C2B | CEO | Mineral |
| C339 | 3C2C | CEO | Mineral |
| C340 | 3C3A | CEO | Mineral |
| C341 | 3C3B | CEO | Mineral |
| C342 | 3C3C | CEO | Mineral |
| C343 | 3C4A | CEO | Mineral |
| C344 | 3C4B | CEO | Mineral |
| C345 | 3C4C | CEO | Mineral |
| C346 | 3C5A | CEO | Mineral |
| C347 | 3C5B | CEO | Mineral |
| C348 | 3C5C | CEO | Mineral |
| C349 | 3C6A | CEO | Mineral |
| C350 | 3C6B | CEO | Mineral |
| C351 | 3C6C | CEO | Mineral |
| C352 | 3C7A | CEO | Mineral |
| C353 | 3C7B | CEO | Mineral |
| C354 | 3C7C | CEO | Mineral |
| C355 | 3C8A | CEO | Mineral |
| C356 | 3C8B | CEO | Mineral |
| C357 | 3C8C | CEO | Mineral |
| C358 | 3C9A | CEO | Mineral |
| C359 | 3C9B | CEO | Mineral |
| C360 | 3C9C | CEO | Mineral |
| C361 | 3A1A | CO | Mineral |
| C362 | 3A1B | CO | Mineral |
| C363 | 3A1C | CO | Mineral |
| C364 | 3A2A | CO | Mineral |
| C365 | 3A2B | CO | Mineral |
| C366 | 3A2C | CO | Mineral |
| C367 | 3A3A | CO | Mineral |
| C368 | 3A3B | CO | Mineral |
| C369 | 3A3C | CO | Mineral |
| C370 | 3B1A | CO | Mineral |
| C371 | 3B1B | CO | Mineral |
| C372 | 3B1C | CO | Mineral |
| C373 | 3B2A | CO | Mineral |
| C374 | 3B2B | CO | Mineral |
| C375 | 3B2C | CO | Mineral |
| C376 | 3B3A | CO | Mineral |
| C377 | 3B3B | CO | Mineral |
| C378 | 3B3C | CO | Mineral |
| C379 | 3C1A | CO | Mineral |
| C380 | 3C1B | CO | Mineral |
| C381 | 3C1C | CO | Mineral |
| C382 | 3C2A | CO | Mineral |
| C383 | 3C2B | CO | Mineral |
| C384 | 3C2C | CO | Mineral |
| C385 | 3C3A | CO | Mineral |
| C386 | 3C3B | CO | Mineral |
| C387 | 3C3C | CO | Mineral |
| C388 | 3C4A | CO | Mineral |
| C389 | 3C4B | CO | Mineral |
| C390 | 3C4C | CO | Mineral |
| C391 | 3C5A | CO | Mineral |
| C392 | 3C5B | CO | Mineral |
| C393 | 3C5C | CO | Mineral |
| C394 | 3C6A | CO | Mineral |
| C395 | 3C6B | CO | Mineral |
| C396 | 3C6C | CO | Mineral |
| C397 | 3C7A | CO | Mineral |
| C398 | 3C7B | CO | Mineral |
| C399 | 3C7C | CO | Mineral |
| C400 | 3C8A | CO | Mineral |
| C401 | 3C8B | CO | Mineral |
| C402 | 3C8C | CO | Mineral |
| C403 | 3C9A | CO | Mineral |
| C404 | 3C9B | CO | Mineral |
| C405 | 3C9C | CO | Mineral |
| C406 | 4A1A | COMP | Mineral |
| C407 | 4A1B | COMP | Mineral |
| C408 | 4A1C | COMP | Mineral |
| C409 | 4A2A | COMP | Mineral |
| C410 | 4A2B | COMP | Mineral |
| C411 | 4A2C | COMP | Mineral |
| C412 | 4A3A | COMP | Mineral |
| C413 | 4A3B | COMP | Mineral |
| C414 | 4A3C | COMP | Mineral |
| C415 | 4B1A | COMP | Mineral |
| C416 | 4B1B | COMP | Mineral |
| C417 | 4B1C | COMP | Mineral |
| C418 | 4B2A | COMP | Mineral |
| C419 | 4B2B | COMP | Mineral |
| C420 | 4B2C | COMP | Mineral |
| C421 | 4B3A | COMP | Mineral |
| C422 | 4B3B | COMP | Mineral |
| C423 | 4B3C | COMP | Mineral |
| C424 | 4C1A | COMP | Mineral |
| C425 | 4C1B | COMP | Mineral |
| C426 | 4C1C | COMP | Mineral |
| C427 | 4C2A | COMP | Mineral |
| C428 | 4C2B | COMP | Mineral |
| C429 | 4C2C | COMP | Mineral |
| C430 | 4C3A | COMP | Mineral |
| C431 | 4C3B | COMP | Mineral |
| C432 | 4C3C | COMP | Mineral |
| C433 | 4C4A | COMP | Mineral |
| C434 | 4C4B | COMP | Mineral |
| C435 | 4C4C | COMP | Mineral |
| C436 | 4C5A | COMP | Mineral |
| C437 | 4C5B | COMP | Mineral |
| C438 | 4C5C | COMP | Mineral |
| C439 | 4C6A | COMP | Mineral |
| C440 | 4C6B | COMP | Mineral |
| C441 | 4C6C | COMP | Mineral |
| C442 | 4C7A | COMP | Mineral |
| C443 | 4C7B | COMP | Mineral |
| C444 | 4C7C | COMP | Mineral |
| C445 | 4C8A | COMP | Mineral |
| C446 | 4C8B | COMP | Mineral |
| C447 | 4C8C | COMP | Mineral |
| C448 | 4C9A | COMP | Mineral |
| C449 | 4C9B | COMP | Mineral |
| C450 | 4C9C | COMP | Mineral |
| C451 | 4A1A | CEO | Mineral |
| C452 | 4A1B | CEO | Mineral |
| C453 | 4A1C | CEO | Mineral |
| C454 | 4A2A | CEO | Mineral |
| C455 | 4A2B | CEO | Mineral |
| C456 | 4A2C | CEO | Mineral |
| C457 | 4A3A | CEO | Mineral |
| C458 | 4A3B | CEO | Mineral |
| C459 | 4A3C | CEO | Mineral |
| C460 | 4B1A | CEO | Mineral |
| C461 | 4B1B | CEO | Mineral |
| C462 | 4B1C | CEO | Mineral |
| C463 | 4B2A | CEO | Mineral |
| C464 | 4B2B | CEO | Mineral |
| C465 | 4B2C | CEO | Mineral |
| C466 | 4B3A | CEO | Mineral |
| C467 | 4B3B | CEO | Mineral |
| C468 | 4B3C | CEO | Mineral |
| C469 | 4C1A | CEO | Mineral |
| C470 | 4C1B | CEO | Mineral |
| C471 | 4C1C | CEO | Mineral |
| C472 | 4C2A | CEO | Mineral |
| C473 | 4C2B | CEO | Mineral |
| C474 | 4C2C | CEO | Mineral |
| C475 | 4C3A | CEO | Mineral |
| C476 | 4C3B | CEO | Mineral |
| C477 | 4C3C | CEO | Mineral |
| C478 | 4C4A | CEO | Mineral |
| C479 | 4C4B | CEO | Mineral |
| C480 | 4C4C | CEO | Mineral |
| C481 | 4C5A | CEO | Mineral |
| C482 | 4C5B | CEO | Mineral |
| C483 | 4C5C | CEO | Mineral |
| C484 | 4C6A | CEO | Mineral |
| C485 | 4C6B | CEO | Mineral |
| C486 | 4C6C | CEO | Mineral |
| C487 | 4C7A | CEO | Mineral |
| C488 | 4C7B | CEO | Mineral |
| C489 | 4C7C | CEO | Mineral |
| C490 | 4C8A | CEO | Mineral |
| C491 | 4C8B | CEO | Mineral |
| C492 | 4C8C | CEO | Mineral |
| C493 | 4C9A | CEO | Mineral |
| C494 | 4C9B | CEO | Mineral |
| C495 | 4C9C | CEO | Mineral |
| C496 | 4A1A | CO | Mineral |
| C497 | 4A1B | CO | Mineral |
| C498 | 4A1C | CO | Mineral |
| C499 | 4A2A | CO | Mineral |
| C500 | 4A2B | CO | Mineral |
| C501 | 4A2C | CO | Mineral |
| C502 | 4A3A | CO | Mineral |
| C503 | 4A3B | CO | Mineral |
| C504 | 4A3C | CO | Mineral |
| C505 | 4B1A | CO | Mineral |
| C506 | 4B1B | CO | Mineral |
| C507 | 4B1C | CO | Mineral |
| C508 | 4B2A | CO | Mineral |
| C509 | 4B2B | CO | Mineral |
| C510 | 4B2C | CO | Mineral |
| C511 | 4B3A | CO | Mineral |
| C512 | 4B3B | CO | Mineral |
| C513 | 4B3C | CO | Mineral |
| C514 | 4C1A | CO | Mineral |
| C515 | 4C1B | CO | Mineral |
| C516 | 4C1C | CO | Mineral |
| C517 | 4C2A | CO | Mineral |
| C518 | 4C2B | CO | Mineral |
| C519 | 4C2C | CO | Mineral |
| C520 | 4C3A | CO | Mineral |
| C521 | 4C3B | CO | Mineral |
| C522 | 4C3C | CO | Mineral |
| C523 | 4C4A | CO | Mineral |
| C524 | 4C4B | CO | Mineral |
| C525 | 4C4C | CO | Mineral |
| C526 | 4C5A | CO | Mineral |
| C527 | 4C5B | CO | Mineral |
| C528 | 4C5C | CO | Mineral |
| C529 | 4C6A | CO | Mineral |
| C530 | 4C6B | CO | Mineral |
| C531 | 4C6C | CO | Mineral |
| C532 | 4C7A | CO | Mineral |
| C533 | 4C7B | CO | Mineral |
| C534 | 4C7C | CO | Mineral |
| C535 | 4C8A | CO | Mineral |
| C536 | 4C8B | CO | Mineral |
| C537 | 4C8C | CO | Mineral |
| C538 | 4C9A | CO | Mineral |
| C539 | 4C9B | CO | Mineral |
| C540 | 4C9C | CO | Mineral |

Polyvinyl Ether (PVE) Lubricant

| | | | |
|---|---|---|---|
| D1 | 1A1A | COMP | PVE |
| D2 | 1A1B | COMP | PVE |
| D3 | 1A1C | COMP | PVE |
| D4 | 1A2A | COMP | PVE |
| D5 | 1A2B | COMP | PVE |
| D6 | 1A2C | COMP | PVE |
| D7 | 1A3A | COMP | PVE |
| D8 | 1A3B | COMP | PVE |
| D9 | 1A3C | COMP | PVE |
| D10 | 1B1A | COMP | PVE |
| D11 | 1B1B | COMP | PVE |
| D12 | 1B1C | COMP | PVE |
| D13 | 1B2A | COMP | PVE |
| D14 | 1B2B | COMP | PVE |
| D15 | 1B2C | COMP | PVE |
| D16 | 1B3A | COMP | PVE |
| D17 | 1B3B | COMP | PVE |
| D18 | 1B3C | COMP | PVE |
| D19 | 1C1A | COMP | PVE |
| D20 | 1C1B | COMP | PVE |
| D21 | 1C1C | COMP | PVE |
| D22 | 1C2A | COMP | PVE |
| D23 | 1C2B | COMP | PVE |
| D24 | 1C2C | COMP | PVE |
| D25 | 1C3A | COMP | PVE |
| D26 | 1C3B | COMP | PVE |
| D27 | 1C3C | COMP | PVE |
| D28 | 1C4A | COMP | PVE |
| D29 | 1C4B | COMP | PVE |
| D30 | 1C4C | COMP | PVE |
| D31 | 1C5A | COMP | PVE |
| D32 | 1C5B | COMP | PVE |
| D33 | 1C5C | COMP | PVE |
| D34 | 1C6A | COMP | PVE |
| D35 | 1C6B | COMP | PVE |
| D36 | 1C6C | COMP | PVE |
| D37 | 1C7A | COMP | PVE |
| D38 | 1C7B | COMP | PVE |
| D39 | 1C7C | COMP | PVE |
| D40 | 1C8A | COMP | PVE |
| D41 | 1C8B | COMP | PVE |
| D42 | 1C8C | COMP | PVE |
| D43 | 1C9A | COMP | PVE |
| D44 | 1C9B | COMP | PVE |
| D45 | 1C9C | COMP | PVE |
| D46 | 1A1A | CEO | PVE |
| D47 | 1A1B | CEO | PVE |
| D48 | 1A1C | CEO | PVE |
| D49 | 1A2A | CEO | PVE |
| D50 | 1A2B | CEO | PVE |
| D51 | 1A2C | CEO | PVE |
| D52 | 1A3A | CEO | PVE |
| D53 | 1A3B | CEO | PVE |
| D54 | 1A3C | CEO | PVE |
| D55 | 1B1A | CEO | PVE |
| D56 | 1B1B | CEO | PVE |
| D57 | 1B1C | CEO | PVE |
| D58 | 1B2A | CEO | PVE |
| D59 | 1B2B | CEO | PVE |
| D60 | 1B2C | CEO | PVE |
| D61 | 1B3A | CEO | PVE |
| D62 | 1B3B | CEO | PVE |
| D63 | 1B3C | CEO | PVE |
| D64 | 1C1A | CEO | PVE |
| D65 | 1C1B | CEO | PVE |
| D66 | 1C1C | CEO | PVE |
| D67 | 1C2A | CEO | PVE |
| D68 | 1C2B | CEO | PVE |
| D69 | 1C2C | CEO | PVE |
| D70 | 1C3A | CEO | PVE |
| D71 | 1C3B | CEO | PVE |
| D72 | 1C3C | CEO | PVE |
| D73 | 1C4A | CEO | PVE |
| D74 | 1C4B | CEO | PVE |
| D75 | 1C4C | CEO | PVE |
| D76 | 1C5A | CEO | PVE |
| D77 | 1C5B | CEO | PVE |
| D78 | 1C5C | CEO | PVE |
| D79 | 1C6A | CEO | PVE |
| D80 | 1C6B | CEO | PVE |
| D81 | 1C6C | CEO | PVE |
| D82 | 1C7A | CEO | PVE |
| D83 | 1C7B | CEO | PVE |
| D84 | 1C7C | CEO | PVE |
| D85 | 1C8A | CEO | PVE |
| D86 | 1C8B | CEO | PVE |
| D87 | 1C8C | CEO | PVE |
| D88 | 1C9A | CEO | PVE |
| D89 | 1C9B | CEO | PVE |
| D90 | 1C9C | CEO | PVE |
| D91 | 1A1A | CO | PVE |
| D92 | 1A1B | CO | PVE |
| D93 | 1A1C | CO | PVE |
| D94 | 1A2A | CO | PVE |
| D95 | 1A2B | CO | PVE |
| D96 | 1A2C | CO | PVE |
| D97 | 1A3A | CO | PVE |
| D98 | 1A3B | CO | PVE |
| D99 | 1A3C | CO | PVE |
| D100 | 1B1A | CO | PVE |
| D101 | 1B1B | CO | PVE |
| D102 | 1B1C | CO | PVE |
| D103 | 1B2A | CO | PVE |
| D104 | 1B2B | CO | PVE |
| D105 | 1B2C | CO | PVE |
| D106 | 1B3A | CO | PVE |
| D107 | 1B3B | CO | PVE |
| D108 | 1B3C | CO | PVE |
| D109 | 1C1A | CO | PVE |
| D110 | 1C1B | CO | PVE |
| D111 | 1C1C | CO | PVE |
| D112 | 1C2A | CO | PVE |
| D113 | 1C2B | CO | PVE |
| D114 | 1C2C | CO | PVE |
| D115 | 1C3A | CO | PVE |
| D116 | 1C3B | CO | PVE |
| D117 | 1C3C | CO | PVE |
| D118 | 1C4A | CO | PVE |
| D119 | 1C4B | CO | PVE |
| D120 | 1C4C | CO | PVE |
| D121 | 1C5A | CO | PVE |
| D122 | 1C5B | CO | PVE |
| D123 | 1C5C | CO | PVE |
| D124 | 1C6A | CO | PVE |
| D125 | 1C6B | CO | PVE |
| D126 | 1C6C | CO | PVE |
| D127 | 1C7A | CO | PVE |
| D128 | 1C7B | CO | PVE |
| D129 | 1C7C | CO | PVE |
| D130 | 1C8A | CO | PVE |
| D131 | 1C8B | CO | PVE |
| D132 | 1C8C | CO | PVE |
| D133 | 1C9A | CO | PVE |
| D134 | 1C9B | CO | PVE |
| D135 | 1C9C | CO | PVE |
| D136 | 2A1A | COMP | PVE |
| D137 | 2A1B | COMP | PVE |
| D138 | 2A1C | COMP | PVE |
| D139 | 2A2A | COMP | PVE |
| D140 | 2A2B | COMP | PVE |
| D141 | 2A2C | COMP | PVE |
| D142 | 2A3A | COMP | PVE |
| D143 | 2A3B | COMP | PVE |
| D144 | 2A3C | COMP | PVE |
| D145 | 2B1A | COMP | PVE |
| D146 | 2B1B | COMP | PVE |
| D147 | 2B1C | COMP | PVE |
| D148 | 2B2A | COMP | PVE |
| D149 | 2B2B | COMP | PVE |
| D150 | 2B2C | COMP | PVE |
| D151 | 2B3A | COMP | PVE |
| D152 | 2B3B | COMP | PVE |
| D153 | 2B3C | COMP | PVE |
| D154 | 2C1A | COMP | PVE |
| D155 | 2C1B | COMP | PVE |
| D156 | 2C1C | COMP | PVE |
| D157 | 2C2A | COMP | PVE |
| D158 | 2C2B | COMP | PVE |
| D159 | 2C2C | COMP | PVE |
| D160 | 2C3A | COMP | PVE |
| D161 | 2C3B | COMP | PVE |
| D162 | 2C3C | COMP | PVE |
| D163 | 2C4A | COMP | PVE |
| D164 | 2C4B | COMP | PVE |
| D165 | 2C4C | COMP | PVE |
| D166 | 2C5A | COMP | PVE |
| D167 | 2C5B | COMP | PVE |
| D168 | 2C5C | COMP | PVE |
| D169 | 2C6A | COMP | PVE |
| D170 | 2C6B | COMP | PVE |
| D171 | 2C6C | COMP | PVE |
| D172 | 2C7A | COMP | PVE |
| D173 | 2C7B | COMP | PVE |
| D174 | 2C7C | COMP | PVE |
| D175 | 2C8A | COMP | PVE |
| D176 | 2C8B | COMP | PVE |
| D177 | 2C8C | COMP | PVE |
| D178 | 2C9A | COMP | PVE |
| D179 | 2C9B | COMP | PVE |
| D180 | 2C9C | COMP | PVE |
| D181 | 2A1A | CEO | PVE |
| D182 | 2A1B | CEO | PVE |
| D183 | 2A1C | CEO | PVE |
| D184 | 2A2A | CEO | PVE |
| D185 | 2A2B | CEO | PVE |
| D186 | 2A2C | CEO | PVE |
| D187 | 2A3A | CEO | PVE |
| D188 | 2A3B | CEO | PVE |
| D189 | 2A3C | CEO | PVE |
| D190 | 2B1A | CEO | PVE |
| D191 | 2B1B | CEO | PVE |
| D192 | 2B1C | CEO | PVE |
| D193 | 2B2A | CEO | PVE |
| D194 | 2B2B | CEO | PVE |
| D195 | 2B2C | CEO | PVE |
| D196 | 2B3A | CEO | PVE |
| D197 | 2B3B | CEO | PVE |
| D198 | 2B3C | CEO | PVE |
| D199 | 2C1A | CEO | PVE |
| D200 | 2C1B | CEO | PVE |
| D201 | 2C1C | CEO | PVE |
| D202 | 2C2A | CEO | PVE |
| D203 | 2C2B | CEO | PVE |
| D204 | 2C2C | CEO | PVE |
| D205 | 2C3A | CEO | PVE |
| D206 | 2C3B | CEO | PVE |
| D207 | 2C3C | CEO | PVE |
| D208 | 2C4A | CEO | PVE |
| D209 | 2C4B | CEO | PVE |
| D210 | 2C4C | CEO | PVE |
| D211 | 2C5A | CEO | PVE |
| D212 | 2C5B | CEO | PVE |
| D213 | 2C5C | CEO | PVE |
| D214 | 2C6A | CEO | PVE |
| D215 | 2C6B | CEO | PVE |
| D216 | 2C6C | CEO | PVE |
| D217 | 2C7A | CEO | PVE |
| D218 | 2C7B | CEO | PVE |
| D219 | 2C7C | CEO | PVE |
| D220 | 2C8A | CEO | PVE |
| D221 | 2C8B | CEO | PVE |
| D222 | 2C8C | CEO | PVE |
| D223 | 2C9A | CEO | PVE |
| D224 | 2C9B | CEO | PVE |
| D225 | 2C9C | CEO | PVE |
| D226 | 2A1A | CO | PVE |
| D227 | 2A1B | CO | PVE |
| D228 | 2A1C | CO | PVE |
| D229 | 2A2A | CO | PVE |
| D230 | 2A2B | CO | PVE |
| D231 | 2A2C | CO | PVE |
| D232 | 2A3A | CO | PVE |
| D233 | 2A3B | CO | PVE |
| D234 | 2A3C | CO | PVE |
| D235 | 2B1A | CO | PVE |
| D236 | 2B1B | CO | PVE |
| D237 | 2B1C | CO | PVE |
| D238 | 2B2A | CO | PVE |
| D239 | 2B2B | CO | PVE |
| D240 | 2B2C | CO | PVE |
| D241 | 2B3A | CO | PVE |
| D242 | 2B3B | CO | PVE |
| D243 | 2B3C | CO | PVE |
| D244 | 2C1A | CO | PVE |
| D245 | 2C1B | CO | PVE |
| D246 | 2C1C | CO | PVE |
| D247 | 2C2A | CO | PVE |
| D248 | 2C2B | CO | PVE |
| D249 | 2C2C | CO | PVE |
| D250 | 2C3A | CO | PVE |
| D251 | 2C3B | CO | PVE |
| D252 | 2C3C | CO | PVE |
| D253 | 2C4A | CO | PVE |
| D254 | 2C4B | CO | PVE |
| D255 | 2C4C | CO | PVE |
| D256 | 2C5A | CO | PVE |
| D257 | 2C5B | CO | PVE |
| D258 | 2C5C | CO | PVE |
| D259 | 2C6A | CO | PVE |
| D260 | 2C6B | CO | PVE |
| D261 | 2C6C | CO | PVE |
| D262 | 2C7A | CO | PVE |
| D263 | 2C7B | CO | PVE |
| D264 | 2C7C | CO | PVE |
| D265 | 2C8A | CO | PVE |
| D266 | 2C8B | CO | PVE |
| D267 | 2C8C | CO | PVE |
| D268 | 2C9A | CO | PVE |
| D269 | 2C9B | CO | PVE |
| D270 | 2C9C | CO | PVE |
| D271 | 3A1A | COMP | PVE |
| D272 | 3A1B | COMP | PVE |
| D273 | 3A1C | COMP | PVE |
| D274 | 3A2A | COMP | PVE |
| D275 | 3A2B | COMP | PVE |
| D276 | 3A2C | COMP | PVE |
| D277 | 3A3A | COMP | PVE |
| D278 | 3A3B | COMP | PVE |
| D279 | 3A3C | COMP | PVE |
| D280 | 3B1A | COMP | PVE |
| D281 | 3B1B | COMP | PVE |
| D282 | 3B1C | COMP | PVE |
| D283 | 3B2A | COMP | PVE |
| D284 | 3B2B | COMP | PVE |
| D285 | 3B2C | COMP | PVE |
| D286 | 3B3A | COMP | PVE |
| D287 | 3B3B | COMP | PVE |
| D288 | 3B3C | COMP | PVE |
| D289 | 3C1A | COMP | PVE |
| D290 | 3C1B | COMP | PVE |
| D291 | 3C1C | COMP | PVE |
| D292 | 3C2A | COMP | PVE |
| D293 | 3C2B | COMP | PVE |
| D294 | 3C2C | COMP | PVE |
| D295 | 3C3A | COMP | PVE |
| D296 | 3C3B | COMP | PVE |
| D297 | 3C3C | COMP | PVE |
| D298 | 3C4A | COMP | PVE |
| D299 | 3C4B | COMP | PVE |
| D300 | 3C4C | COMP | PVE |
| D301 | 3C5A | COMP | PVE |
| D302 | 3C5B | COMP | PVE |
| D303 | 3C5C | COMP | PVE |
| D304 | 3C6A | COMP | PVE |
| D305 | 3C6B | COMP | PVE |
| D306 | 3C6C | COMP | PVE |
| D307 | 3C7A | COMP | PVE |
| D308 | 3C7B | COMP | PVE |
| D309 | 3C7C | COMP | PVE |
| D310 | 3C8A | COMP | PVE |
| D311 | 3C8B | COMP | PVE |
| D312 | 3C8C | COMP | PVE |
| D313 | 3C9A | COMP | PVE |
| D314 | 3C9B | COMP | PVE |
| D315 | 3C9C | COMP | PVE |
| D316 | 3A1A | CEO | PVE |
| D317 | 3A1B | CEO | PVE |
| D318 | 3A1C | CEO | PVE |
| D319 | 3A2A | CEO | PVE |
| D320 | 3A2B | CEO | PVE |
| D321 | 3A2C | CEO | PVE |
| D322 | 3A3A | CEO | PVE |
| D323 | 3A3B | CEO | PVE |
| D324 | 3A3C | CEO | PVE |
| D325 | 3B1A | CEO | PVE |
| D326 | 3B1B | CEO | PVE |
| D327 | 3B1C | CEO | PVE |
| D328 | 3B2A | CEO | PVE |
| D329 | 3B2B | CEO | PVE |
| D330 | 3B2C | CEO | PVE |
| D331 | 3B3A | CEO | PVE |
| D332 | 3B3B | CEO | PVE |
| D333 | 3B3C | CEO | PVE |
| D334 | 3C1A | CEO | PVE |
| D335 | 3C1B | CEO | PVE |
| D336 | 3C1C | CEO | PVE |
| D337 | 3C2A | CEO | PVE |
| D338 | 3C2B | CEO | PVE |
| D339 | 3C2C | CEO | PVE |
| D340 | 3C3A | CEO | PVE |
| D341 | 3C3B | CEO | PVE |
| D342 | 3C3C | CEO | PVE |
| D343 | 3C4A | CEO | PVE |
| D344 | 3C4B | CEO | PVE |
| D345 | 3C4C | CEO | PVE |
| D346 | 3C5A | CEO | PVE |
| D347 | 3C5B | CEO | PVE |
| D348 | 3C5C | CEO | PVE |
| D349 | 3C6A | CEO | PVE |
| D350 | 3C6B | CEO | PVE |
| D351 | 3C6C | CEO | PVE |
| D352 | 3C7A | CEO | PVE |
| D353 | 3C7B | CEO | PVE |
| D354 | 3C7C | CEO | PVE |
| D355 | 3C8A | CEO | PVE |
| D356 | 3C8B | CEO | PVE |
| D357 | 3C8C | CEO | PVE |
| D358 | 3C9A | CEO | PVE |
| D359 | 3C9B | CEO | PVE |
| D360 | 3C9C | CEO | PVE |
| D361 | 3A1A | CO | PVE |
| D362 | 3A1B | CO | PVE |
| D363 | 3A1C | CO | PVE |
| D364 | 3A2A | CO | PVE |
| D365 | 3A2B | CO | PVE |
| D366 | 3A2C | CO | PVE |
| D367 | 3A3A | CO | PVE |
| D368 | 3A3B | CO | PVE |
| D369 | 3A3C | CO | PVE |
| D370 | 3B1A | CO | PVE |
| D371 | 3B1B | CO | PVE |
| D372 | 3B1C | CO | PVE |
| D373 | 3B2A | CO | PVE |
| D374 | 3B2B | CO | PVE |
| D375 | 3B2C | CO | PVE |
| D376 | 3B3A | CO | PVE |
| D377 | 3B3B | CO | PVE |
| D378 | 3B3C | CO | PVE |
| D379 | 3C1A | CO | PVE |
| D380 | 3C1B | CO | PVE |
| D381 | 3C1C | CO | PVE |
| D382 | 3C2A | CO | PVE |
| D383 | 3C2B | CO | PVE |
| D384 | 3C2C | CO | PVE |
| D385 | 3C3A | CO | PVE |
| D386 | 3C3B | CO | PVE |
| D387 | 3C3C | CO | PVE |
| D388 | 3C4A | CO | PVE |
| D389 | 3C4B | CO | PVE |
| D390 | 3C4C | CO | PVE |
| D391 | 3C5A | CO | PVE |
| D392 | 3C5B | CO | PVE |
| D393 | 3C5C | CO | PVE |
| D394 | 3C6A | CO | PVE |
| D395 | 3C6B | CO | PVE |
| D396 | 3C6C | CO | PVE |
| D397 | 3C7A | CO | PVE |
| D398 | 3C7B | CO | PVE |
| D399 | 3C7C | CO | PVE |
| D400 | 3C8A | CO | PVE |
| D401 | 3C8B | CO | PVE |
| D402 | 3C8C | CO | PVE |
| D403 | 3C9A | CO | PVE |
| D404 | 3C9B | CO | PVE |
| D405 | 3C9C | CO | PVE |
| D406 | 4A1A | COMP | PVE |
| D407 | 4A1B | COMP | PVE |
| D408 | 4A1C | COMP | PVE |
| D409 | 4A2A | COMP | PVE |
| D410 | 4A2B | COMP | PVE |
| D411 | 4A2C | COMP | PVE |
| D412 | 4A3A | COMP | PVE |
| D413 | 4A3B | COMP | PVE |
| D414 | 4A3C | COMP | PVE |
| D415 | 4B1A | COMP | PVE |
| D416 | 4B1B | COMP | PVE |
| D417 | 4B1C | COMP | PVE |
| D418 | 4B2A | COMP | PVE |
| D419 | 4B2B | COMP | PVE |
| D420 | 4B2C | COMP | PVE |
| D421 | 4B3A | COMP | PVE |
| D422 | 4B3B | COMP | PVE |
| D423 | 4B3C | COMP | PVE |
| D424 | 4C1A | COMP | PVE |
| D425 | 4C1B | COMP | PVE |
| D426 | 4C1C | COMP | PVE |
| D427 | 4C2A | COMP | PVE |
| D428 | 4C2B | COMP | PVE |
| D429 | 4C2C | COMP | PVE |
| D430 | 4C3A | COMP | PVE |
| D431 | 4C3B | COMP | PVE |
| D432 | 4C3C | COMP | PVE |
| D433 | 4C4A | COMP | PVE |
| D434 | 4C4B | COMP | PVE |
| D435 | 4C4C | COMP | PVE |
| D436 | 4C5A | COMP | PVE |
| D437 | 4C5B | COMP | PVE |
| D438 | 4C5C | COMP | PVE |
| D439 | 4C6A | COMP | PVE |
| D440 | 4C6B | COMP | PVE |
| D441 | 4C6C | COMP | PVE |
| D442 | 4C7A | COMP | PVE |
| D443 | 4C7B | COMP | PVE |
| D444 | 4C7C | COMP | PVE |
| D445 | 4C8A | COMP | PVE |
| D446 | 4C8B | COMP | PVE |
| D447 | 4C8C | COMP | PVE |
| D448 | 4C9A | COMP | PVE |
| D449 | 4C9B | COMP | PVE |
| D450 | 4C9C | COMP | PVE |
| D451 | 4A1A | CEO | PVE |
| D452 | 4A1B | CEO | PVE |
| D453 | 4A1C | CEO | PVE |
| D454 | 4A2A | CEO | PVE |
| D455 | 4A2B | CEO | PVE |
| D456 | 4A2C | CEO | PVE |
| D457 | 4A3A | CEO | PVE |
| D458 | 4A3B | CEO | PVE |
| D459 | 4A3C | CEO | PVE |
| D460 | 4B1A | CEO | PVE |
| D461 | 4B1B | CEO | PVE |
| D462 | 4B1C | CEO | PVE |
| D463 | 4B2A | CEO | PVE |
| D464 | 4B2B | CEO | PVE |
| D465 | 4B2C | CEO | PVE |
| D466 | 4B3A | CEO | PVE |
| D467 | 4B3B | CEO | PVE |
| D468 | 4B3C | CEO | PVE |
| D469 | 4C1A | CEO | PVE |
| D470 | 4C1B | CEO | PVE |
| D471 | 4C1C | CEO | PVE |
| D472 | 4C2A | CEO | PVE |
| D473 | 4C2B | CEO | PVE |
| D474 | 4C2C | CEO | PVE |
| D475 | 4C3A | CEO | PVE |
| D476 | 4C3B | CEO | PVE |
| D477 | 4C3C | CEO | PVE |
| D478 | 4C4A | CEO | PVE |
| D479 | 4C4B | CEO | PVE |
| D480 | 4C4C | CEO | PVE |
| D481 | 4C5A | CEO | PVE |
| D482 | 4C5B | CEO | PVE |
| D483 | 4C5C | CEO | PVE |
| D484 | 4C6A | CEO | PVE |
| D485 | 4C6B | CEO | PVE |
| D486 | 4C6C | CEO | PVE |
| D487 | 4C7A | CEO | PVE |
| D488 | 4C7B | CEO | PVE |
| D489 | 4C7C | CEO | PVE |
| D490 | 4C8A | CEO | PVE |
| D491 | 4C8B | CEO | PVE |
| D492 | 4C8C | CEO | PVE |
| D493 | 4C9A | CEO | PVE |
| D494 | 4C9B | CEO | PVE |
| D495 | 4C9C | CEO | PVE |
| D496 | 4A1A | CO | PVE |
| D497 | 4A1B | CO | PVE |
| D498 | 4A1C | CO | PVE |
| D499 | 4A2A | CO | PVE |
| D500 | 4A2B | CO | PVE |
| D501 | 4A2C | CO | PVE |
| D502 | 4A3A | CO | PVE |
| D503 | 4A3B | CO | PVE |
| D504 | 4A3C | CO | PVE |
| D505 | 4B1A | CO | PVE |
| D506 | 4B1B | CO | PVE |
| D507 | 4B1C | CO | PVE |
| D508 | 4B2A | CO | PVE |
| D509 | 4B2B | CO | PVE |
| D510 | 4B2C | CO | PVE |
| D511 | 4B3A | CO | PVE |
| D512 | 4B3B | CO | PVE |
| D513 | 4B3C | CO | PVE |
| D514 | 4C1A | CO | PVE |
| D515 | 4C1B | CO | PVE |
| D516 | 4C1C | CO | PVE |
| D517 | 4C2A | CO | PVE |
| D518 | 4C2B | CO | PVE |
| D519 | 4C2C | CO | PVE |
| D520 | 4C3A | CO | PVE |
| D521 | 4C3B | CO | PVE |
| D522 | 4C3C | CO | PVE |
| D523 | 4C4A | CO | PVE |
| D524 | 4C4B | CO | PVE |
| D525 | 4C4C | CO | PVE |
| D526 | 4C5A | CO | PVE |
| D527 | 4C5B | CO | PVE |
| D528 | 4C5C | CO | PVE |
| D529 | 4C6A | CO | PVE |
| D530 | 4C6B | CO | PVE |
| D531 | 4C6C | CO | PVE |
| D532 | 4C7A | CO | PVE |
| D533 | 4C7B | CO | PVE |
| D534 | 4C7C | CO | PVE |
| D535 | 4C8A | CO | PVE |
| D536 | 4C8B | CO | PVE |
| D537 | 4C8C | CO | PVE |
| D538 | 4C9A | CO | PVE |
| D539 | 4C9B | CO | PVE |
| D540 | 4C9C | CO | PVE |

The present invention includes methods of heating and/or cool articles and/or devices during the manufacture and/or operation thereof comprising:
(a) providing an electronic component, article and/or device which is being manufactured and/or being operated for its intended purpose; and
(b) during at least a portion of said manufacturing process or operating process, transferring heat to and/or from said electronic component, article or device by directly or indirectly transferring heat between said electronic component, article and/or device and a refrigerant fluid according to the present invention, including any of Refrigerants 1-4.

Heat transfer methods according to this paragraph are sometimes referred to herein for convenience as Heat Transfer Methods 1.

The present invention also includes methods of heating and/or cooling of components, articles and/or devices during the manufacture and/or operation thereof comprising:
(a) providing an electronic component, article and/or device which is being manufactured and/or being operated for its intended purpose; and
(b) during at least a portion of said manufacturing and/or operating process, transferring heat to and/or from said electronic component, article and/or device by directly transferring heat between said electronic component, article or device and a refrigerant fluid comprising according to the present invention, including any of Refrigerants 1-4.

Heat transfer methods according to this paragraph are sometimes referred to herein for convenience as Heat Transfer Methods 2A.

The present invention also includes methods of heating and/or cooling of components, articles and/or devices during the manufacture and/or operation thereof comprising:
(a) providing an electronic component, article and/or device which is being manufactured and/or being operated for its intended purpose; and
(b) during at least a portion of said manufacturing and/or operating process, transferring heat to and/or from said electronic component, article and/or device by direct sensible heat transfer between said electronic component, article and/or device and a refrigerant fluid according to the present invention, including any of Refrigerants 1-4.

Heat transfer methods according to this paragraph are sometimes referred to herein for convenience as Heat Transfer Methods 2B.

The present invention also includes methods of heating and/or cooling of components, articles and/or devices during the manufacture and/or operation thereof comprising:
(a) providing an electronic component, article and/or device which is being manufactured and/or being operated for its intended purpose; and
(b) during at least a portion of said manufacturing and/or operating process, transferring heat to and/or from said electronic component, article and/or device by direct latent heat transfer between said electronic component, article and/or device and a refrigerant fluid according to the present invention, including any of Refrigerants 1-4.

Heat transfer methods according to this paragraph are sometimes referred to herein for convenience as Heat Transfer Methods 2C.

The present invention also includes methods of heating and/or cooling of components, articles and/or devices during the manufacture and/or operation thereof comprising:
(c) providing an electronic component, article and/or device which is being manufactured and/or being operated for its intended purpose; and
(d) during at least a portion of said manufacturing and/or operating process, transferring heat to and/or from said electronic component, article and/or device by a both direct latent heat transfer and direct sensible heat transfer between said electronic component, article and/or device and a refrigerant composition according to the present invention, including any of Refrigerants 1-4.

Heat transfer methods according to this paragraph are sometimes referred to herein for convenience as Heat Transfer Methods 2D.

The present invention also includes electronic components, devices, articles and systems which are in thermal contact, either directly or indirectly, with Refrigerant 1. Electronic components, devices, articles and systems according to this paragraph are sometimes referred to herein for convenience as Electronic 1A.

The present invention also includes electronic components, devices, articles and systems which are in direct thermal contact with Refrigerant 1. Electronic components, devices, articles and systems according to this paragraph are sometimes referred to herein for convenience as Electronic 1B.

The present invention also includes electronic components, devices, articles and systems which are in thermal contact, either directly or indirectly, with Refrigerant 2. Electronic components, devices, articles and systems according to this paragraph are sometimes referred to herein for convenience as Electronic 2A.

The present invention also includes electronic components, devices, articles and systems which are in direct thermal contact with Refrigerant 2. Electronic components, devices, articles and systems according to this paragraph are sometimes referred to herein for convenience as Electronic 2B.

The present invention also includes electronic components, devices, articles and systems which are in thermal contact, either directly or indirectly, with Refrigerant 3. Electronic components, devices, articles and systems according to this paragraph are sometimes referred to herein for convenience as Electronic 3A.

The present invention also includes electronic components, devices, articles and systems which are in direct thermal contact with Refrigerant 3. Electronic components, devices, articles and systems according to this paragraph are sometimes referred to herein for convenience as Electronic 3B.

The present invention also includes electronic components, devices, articles and systems which are in thermal contact, either directly or indirectly, with Refrigerant 4. Electronic components, devices, articles and systems according to this paragraph are sometimes referred to herein for convenience as Electronic 4A.

The present invention also includes electronic components, devices, articles and systems which are in direct thermal contact with Refrigerant 4. Electronic components, devices, articles and systems according to this paragraph are sometimes referred to herein for convenience as Electronic 4B.

The present invention also includes electronic components, devices, articles and systems which are in thermal contact, either directly or indirectly, with a Refrigerant 5. Electronic components, devices, articles and systems according to this paragraph are sometimes referred to herein for convenience as Electronic 5A.

The present invention also includes electronic components, devices, articles and systems which are in direct thermal contact with Refrigerant 5. Electronic components, devices, articles and systems according to this paragraph are sometimes referred to herein for convenience as Electronic 5B.

The terms “secBuFO” and “tBuFO” as used herein, encompass the compounds identified in, and having the physical properties identified in, the following Table 3, it being understood that the numeric values are proceeded by the term “about.”

TABLE 3

| | | Physical Properties | | | |
|---|---|---|---|---|---|
| | Boiling | Dielectric | Dissipation | | |
| | point | Constant | Factor | | |
| Molecule | (° C.) | (20 GHz) | (20 GHz) | GWP | ODP |
| trans-1,3,4,4,5,5,5-heptafluoro- | 55.5-55.9 | 2.11 | — | <50 | 0 |
| 3-(trifluoromethyl)pent-1-ene | | | | | |
| (trans-secBuFO) | | | | | |
| cis-1,3,4,4,5,5,5-heptafluoro-3- | 79.27 | 6.48 | — | <50 | 0 |
| (trifluoromethyl)pent-1-ene | | | | | |
| (cis-secBuFO) | | | | | |
| trans-1,4,4,4-tetrafluoro-3,3- | 57.9 | 2.15 | — | <50 | 0 |
| bis(trifluoromethyl)but-1-ene | | | | | |
| (trans-tBuFO) | | | | | |
| cis-1,4,4,4-tetrafluoro-3,3- | 76.19 | 6.51 | — | <50 | 0 |
| bis(trifluoromethyl)but-1-ene | | | | | |
| (cis-tBuFO) | | | | | |

The novel compounds of the present invention and the compositions of the present invention, including Refrigerants 1-4 and/or Heat Transfer Compositions A-D, may be used for a variety of applications including but not limited to: (1) refrigerants for use in a variety of heat transfer applications (including in thermal management systems and methods) and (2) power cycle working fluids. The novel compounds of the present invention may also be used as starting materials for producing other organofluorine compounds.

### III. Methods of Synthesis

The present invention also provides methods of synthesis for 1,3,4,4,5,5,5-heptafluoro-3-(trifluoromethyl)pent-1-ene (secBuFO) and 1,4,4,4-tetrafluoro-3,3-bis(trifluoromethyl)but-1-ene (tBuFO).

Scheme 1 as described below is an example of the synthesis of secBuFO:

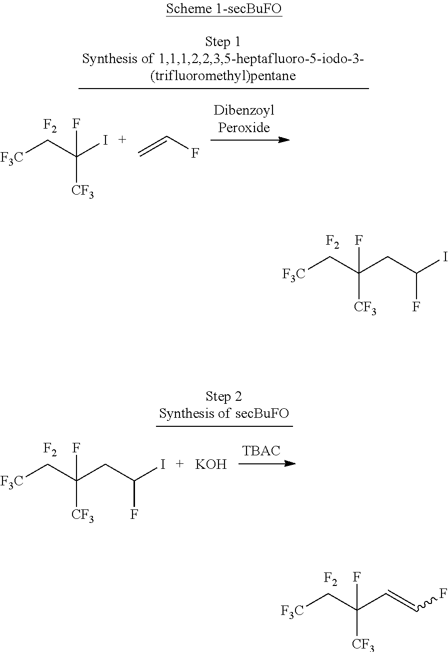

In Step 1 of Scheme 1, 2-iodononafluorobutane is reacted with vinylidene fluoride in the presence of a catalyst, such as dibenzoyl peroxide, to yield 1,1,1,2,2,3,5-heptafluoro-5-iodo-3-(trifluoromethyl)pentane intermediate.

In Step 2 of Scheme 1, the 1,1,1,2,2,3,5-heptafluoro-5-iodo-3-(trifluoromethyl)pentane intermediate is reacted with a base in the presence of a catalyst to yield trans and cis 1,3,4,4,5,5,5-heptafluoro-3-(trifluoromethyl)pent-1-ene in molar ratio of about 3:1.

Further details of this synthesis are set forth below and in Example 1.

Scheme 2 described below is an example of the synthesis of tBuFO:

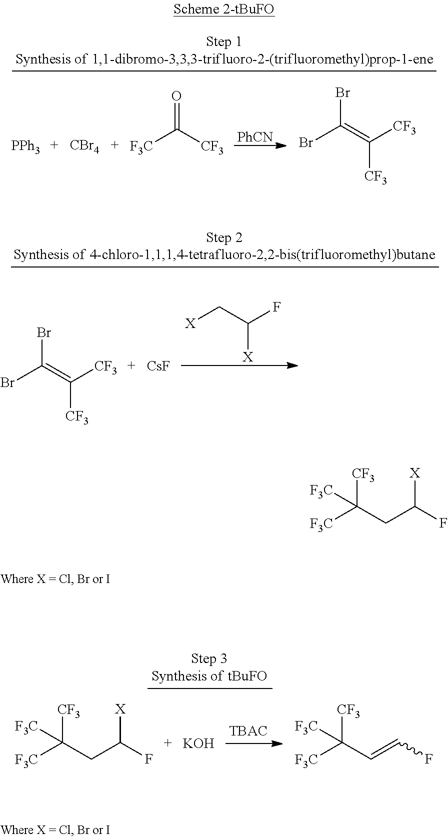

In step 1 of Scheme 2, hexafluoroacetone is reacted with carbon tetrabromide (CBr₄) and triphenyl phosphine to yield 1,1-dibromo-3,3,3-trifluoro-2-(trifluoromethyl)prop-1-ene which, in step 2, is reacted with cesium fluoride (CsF) to yield 4-halo-1,1,1,4-tetrafluoro-2,2-bis(trifluoromethyl)butane. In Step 3 of Scheme 2, the 4-halo-1,1,1,4-tetrafluoro-2,2-bis(trifluoromethyl)butane intermediate is reacted with a base in the presence of a catalyst to yield trans and cis 1,4,4,4-tetrafluoro-3,3-bis(trifluoromethyl)but-1-ene in molar ratio of about 3:1.

Further details of this synthesis are set forth below and in Example 2.

The last step of each of the synthesis methods of Scheme 1 and Scheme 2 involves an HX (X=Cl, Br or I) elimination reaction from an alkyl-halide starting material in the presence of base and a catalyst to give the desired product. In some examples, the reaction involves drop-wise addition of the alkyl halide starting material into a mixture of solvent, base, and catalyst. Then the desired isomeric mixture may be collected from the reaction mixture.

In the last step of each of the synthesis methods of Scheme 1 and Scheme 2, the base may be any suitable organic or inorganic base such as KOH, NaOH, or LiOH, for example.

In the last step of each of the synthesis methods of Scheme 1 and Scheme 2, the catalyst may be a phase transfer catalyst such as tetrabutylammonium chloride (TBAC), tetraethylammonium bromide (TEAB), benzyltriethylammonium chloride (BTEAC), or hexadecyltributylphosphonium bromide.

In the last step of each of the synthesis methods of Scheme 1 and Scheme 2, the reaction temperature may be as low as 20° C., 30° C., 40° C., 50° C., 60° C., or as high as 70° C., 80° C., 90° C., or 100° C., or within any range encompassed by any two of the foregoing values as endpoints. For example, the reaction temperature may be from 50° C. to 100° C., or from 50° C. to 80° C.

After complete addition of the alkyl halide starting material to the reaction mixture, the reaction may be stirred for 1 hour, 2 hours, 3 hours, 4 hours, 5 hours, 6 hours, 7 hours, 8 hours, 9 hours, 10 hours, or within any range encompassed by any two of the foregoing values as endpoints, for example, from 1 hour to 10 hours, or from 3 hours to 7 hours.

During the reaction, the desired isomeric mixture may be in the gas phase, i.e., it may evaporate from the reaction mixture once it is formed. In order to collect the product, the reaction vessel may be outfitted with a distilling head in order to condense the product to a liquid.

In both Scheme 1 and Scheme 2, fractional distillation is performed to isolate the desired trans and cis isomers.

The synthesis methods of each of Scheme 1 and Scheme 2 result in final reaction products that are highly pure in the desired compound.

For instance, as relating to scheme 1, when the trans isomer of secBuFO is the desired reaction product resulting from scheme 1, the desired reaction product may be present at a relatively high weight percent, as compared to one or more impurities that may also be present in the resulting reaction product composition. Specifically, the trans isomer of secBuFO resulting from Scheme 1 may be present in an amount as low as 95.0 wt. %, 96.0 wt. %, 97.0 wt. % or 98.0 wt. %, or as high as 98.5 wt. %, 99.0%, 99.50 wt. %, 99.90%, or 99.95 wt. %, or greater than 99.95 wt. %, as based upon the total weight of the reaction product composition including the trans secBuFO and the one or more impurities, or between any of the foregoing values used as endpoints. For example, the trans secBuFO may be present in an amount between 95.0 wt. % and 99.95 wt. %, 98.5 wt. % and 99.95 wt. %, 99.0 wt. % and 99.95 wt. %, 99.5 wt. % and 99.95 wt. %, 99.90 wt. % and 99.95 wt. %, and/or about 99.95 wt. % or greater, as based upon the total weight of the reaction product including the trans secBuFO and the one or more impurities.

The one or more impurities of the reaction product composition may include compounds such as the cis isomer of secBuFO, partially fluorinated benzene compounds such as C6H2F8 Isomers including 1,2,3,4,5,6-Octafluoro-1,4-dihydrobenzene, 1,2,3,4,5,6-Octafluoro-1,2-dihydrobenzene, 1,2,3,4,5,6-Octafluoro-1,3-dihydrobenzene and the like, and perfluorinated alkyl compounds such as perfluorinated alkyl iodide.

For instance, as relating to scheme 1, when the cis isomer of secBuFO is the desired reaction product resulting from scheme 1, the desired reaction product may be present at a relatively high weight percent, as compared to one or more impurities that may also be present in the resulting reaction product composition. Specifically, the cis isomer of secBuFO resulting from Scheme 1 may be present in an amount as low as 95.0 wt. %, 96.0 wt. %, or 97.0 wt. % or 98.0 wt. %, or as high as 98.5 wt. %, 99.0%, 99.5 wt. %, 99.90%, or 99.95 wt. %, or greater than 99.95 wt. %, as based upon the total weight of the reaction product composition including the cis secBuFO and the one or more impurities, or between any of the foregoing values used as endpoints. For example, the cis secBuFO may be present in an amount between 95.0 wt. % and 99.95 wt. %, 98.5 wt. % and 99.95 wt. %, 99.0 wt. % and 99.95 wt. %, 99.5 wt. % and 99.95 wt. %, 99.90 wt. % and 99.95 wt. %, and/or about 99.95 wt. % or greater, as based upon the total weight of the reaction product including the cis secBuFO and the one or more impurities.

The one or more impurities of the reaction product composition may include compounds such as the trans isomer of secBuFO, as well as additional impurities

As relating to scheme 2, when the trans isomer of tBuFO is the desired reaction product resulting from scheme 2, the desired reaction product may be present at a relatively high weight percent, as compared to one or more impurities that may also be present in the resulting reaction product composition. Specifically, the trans isomer of tBuFO resulting from Scheme 2 may be present in an amount as low as 95.0 wt. %, 96.0 wt. %, or 97.0 wt. %, or 98.0 wt. %, or as high as 98.5 wt. %, 99.0%, 99.5 wt. %, 99.9%, 99.94 wt. %, or 99.95 wt. %, or greater than 99.95 wt. %, as based upon the total weight of the reaction product composition including the trans tBuFO and the one or more impurities, or between any of the foregoing values used as endpoints. For example, the trans tBuFO may be present in an amount between 95.0 wt. % and 99.95 wt. %, 98.0 wt. % and 99.95 wt. %, 98.5 wt. % and 99.95 wt. %, 99.5 wt. % and 99.95 wt. %, 99.9 wt. % and 99.95 wt. %, and/or 99.94 wt. % and 99.95 wt. %, or greater, as based upon the total weight of the reaction product including the trans tBuFO and the one or more impurities.

The one or more impurities of the reaction product composition may include compounds such as the cis isomer of tBuFO, as well as additional impurities, such as water.

As relating to scheme 2, when the cis isomer of tBuFO is the desired reaction product resulting from scheme 2, the desired reaction product may be present at a relatively high weight percent, as compared to one or more impurities that may also be present in the resulting reaction product composition. Specifically, the cis isomer of tBuFO resulting from Scheme 2 may be present in an amount as low as 95.0 wt. %, 96.0 wt. %, or 97.0 wt. %, or 98.0 wt. %, or as high as 98.5 wt. %, 99.0%, 99.5 wt. %, 99.9%, 99.94 wt. %, or 99.95 wt. %, or greater than 99.95 wt. %, as based upon the total weight of the reaction product composition including the cis tBuFO and the one or more impurities, or between any of the foregoing values used as endpoints. For example, the cis tBuFO may be present in an amount between 95.0 wt. % and 99.95 wt. %, 98.0 wt. % and 99.95 wt. %, 98.5 wt. % and 99.95 wt. %, 99.5 wt. % and 99.95 wt. %, 99.9 wt. % and 99.95 wt. %, and/or 99.94 wt. % and 99.95 wt. %, or greater, as based upon the total weight of the reaction product including the cis tBuFO and the one or more impurities.

The one or more impurities of the reaction product composition may include compounds such as the trans isomer of tBuFO, as well as additional impurities, such as water.

### IIV. Heat Transfer Applications

The present invention includes heating and/or cooling generally, and particularly and preferably of electronics both during the manufacture thereof and during operations. Furthermore, the heating and cooling steps can include both direct heat transfer and indirect heat transfer. As used herein, the term “direct heat transfer” refers to contact between the refrigerant compositions of the present invention, including each of Refrigerants 1-4, and the electronic component, article or device being cooled and/or heated. In this context, direct contact is intended to include contact with either the electronic portion of the component itself and/or a substrate or other platform (and extended surfaces thereof) on which electronic components are mounted.

The preferred refrigerants of the present invention, including each of Refrigerants 1-4, are particularly and unexpectedly advantageous in heat transfer uses and methods, including especially uses and methods relating to cooling and/or heating of electronic components, articles and devices, and especially in preferred embodiments comprising heating and/or cooling electronic components, articles or devices which include one or more semiconductors and/or one or more batteries. These advantages stem in part from the preferred compositions, including particularly, each of Refrigerants 1-4, because of applicants' recognition that these refrigerants have advantageous properties for these applications, including relatively low vapor pressure, relatively high boiling point, relatively low dielectric constant and nonflammability.

For the purposes of convenience but not by way of limitation, preferred heat transfer applications which involve manufacture of the electronic component, article or device and heat transfer applications involving heating and/or cooling while the electronic component, article or device are operating are discussed under separate headings below, it being understood however that the invention of a particular embodiment below with respect to heating and/or cooling during manufacture will frequently have application to some extent also in connection with heating and/or cooling of electronic devices during operations, and visa vera. Accordingly, therefore, reference herein to thermal management systems (TMSs) and thermal management methods (TMMs) is understood to represent systems, methods and uses consistent with all types of heating and/or cooling of electronic components, articles or devices.

### IIV.A. Thermal Management Systems, Methods and Uses

The present invention encompasses various methods, processes and uses of the compounds and compositions of the present invention, including refrigerants of the present invention, including each of Refrigerants 1-4, in TMSs and TMMs used to maintain or help maintain a component, article or device (preferably an electronic component, article or device (including batteries) or fluid within a certain temperature range, particularly as that component, article or device or fluid is operating according to its intended purpose and/or during the manufacture of the component, device or article, particularly during the manufacture an electronic device or component (such as a semiconductor wafer or integrated circuit chip etching). For example, the refrigerants of the present invention, including each of Refrigerants 1-4, may be used to keep the temperature of a component below a defined upper and/or above a defined lower temperature, including during the processing/manufacture thereof and/or during the operation thereof for its intended purpose.

The disclosure includes refrigerants of the present disclosure, including each of Refrigerants 1-4, in which the refrigerant is non-flammable.

The invention includes refrigerants of the present invention, including each of Refrigerants 1-4, in which the refrigerant has a dielectric constant less than 3 at 20 GHz.

The invention includes refrigerants of the present invention, including each of Refrigerants 1-4, in which the refrigerant has a dielectric constant less than 2.5 at 20 GHz.

The invention includes refrigerants of the present invention, including each of Refrigerants 1-4, in which the refrigerant has a dielectric constant less than 3 at 20 GHz; (ii) has a boiling point of from about 35° C. to about 80° C.; and (iii) is non-flammable.

The invention includes refrigerants of the present invention, including each of Refrigerant 1, in which the refrigerant has acceptable toxicity.

The invention includes refrigerants of the present invention, including Refrigerants 1-4, in which the refrigerant has a boiling point of from about 35° C. to about 80° C.

Table 4 below defines some preferred uses of the present refrigerants and methods of using the present refrigerants. The first column of Table 4 below identifies and defines the use number (e.g., 1A-9H3) where, in column 2, the refrigerant is identified as based upon the refrigerant definitions in Table 1. Here, each use No. encompasses each individual refrigerant defined in Column 2 regarding the particularly application, such as use 1A encompassing the use of each of refrigerants 1A1-1C3E in the given context. The fourth column denotes the Heat transfer type column regarding the nature of the heat transfer performed by the refrigerant (e.g., single phase, sensible heat, and/or phase change). The fourth column denotes that particular application of the refrigerant. The final sixth column designates the operational temperature range associated with the refrigerant's use. The designation “NR” is understood to mean that the component or property is not required (but may be present) by the use defined in each particular row of the table.

Lengthy table referenced here
US20250297152A1-20250925-T00001
Please refer to the end of the specification for access instructions.

Various details, examples and descriptions of these and other specific applications, uses and methods are discussed below:

### Heat Transfer Compositions

As mentioned above, the present invention provides various methods, processes, and uses of the refrigerants of the present invention, including each of Refrigerants 1-4, to transmit heat from one location to another (or from one body, or article or fluid to another body, article or fluid). For example, the refrigerants of the present invention, including each of Refrigerants 1-4, may be used to keep the temperature of a device below a defined upper and/or above a defined lower temperature. In another example, the present refrigerants, which include refrigerants of the present invention, including each of Refrigerants 1-4, may be used for energy conversion, as in the capture of waste heat from industrial or other processes and the conversion to electrical or mechanical energy.

### Thermal Management Systems and Methods

One important category of use of the present refrigerants is in connection with thermal management systems and methods. Accordingly, the present invention encompasses various methods, processes and uses of the compounds and compositions of the present invention, including refrigerants of the present invention, including each of Refrigerants 1-4, in thermal management systems (hereinafter sometimes referred to as TMS) which operated to maintain an article or device (preferably an electronic component, device, article (including a battery)) or fluid within a certain temperature range, particularly as that article, device or fluid is operating according to its intended purpose and/or during the manufacture of a device or article, particularly during the manufacture an electronic device or component (such as a semiconductor wafer or integrated circuit chip). For example, the TMSs may keep the temperature of a device below a defined upper and/or above a defined lower temperature, including during the processing/manufacture thereof.

As discussed above, the refrigerants of the present invention, including each of Refrigerants 1-4, can be advantageously used in a method or device or system of cooling and/or heating in an electronic device and/or used in a method or device or system for manufacture of an electronic component, device or article (such as a semiconductor wafer or integrated circuit chip).

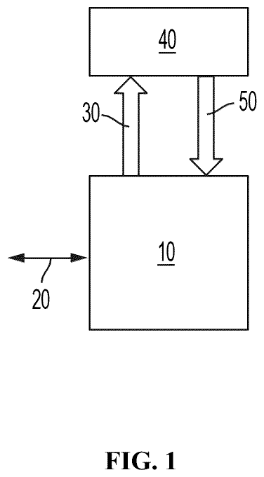

Preferred embodiments of the present thermal management methods are now discussed in connection with FIG. 1. An operating electronic device is shown schematically as 10 having a source of electrical energy and/or signals 20 flowing into and/or out of the device 10 and which generates heat as a result of its operation based on the electrical energy and/or signals 20. The refrigerants of the present invention, including each of Refrigerants 1-4, are provided in thermal contact with the operating device 10 such that it removes heat, represented by the out flowing arrow 30. Heat is removed from the operating electronic device by sensible heat being added to the liquid thermal management fluid of the present invention (i.e., increasing the temperature of the liquid), or by causing a phase change in the thermal management liquid (i.e., vaporizing the liquid) or a combination of these. In preferred embodiments, the methods provide a supply of the refrigerants of the present invention, including each of Refrigerants 1-4, to the device 10 such that the flow of heat from the device 10 through the present refrigerant 30 maintains the operating electrical device at or within a preferred operating temperature range. In preferred embodiments, the preferred operating temperature range of the electrical device is from about 70° C. to about 150° C., and even more preferably from about 70° C. to about 120° C., and the flow of heat 30 from the device 10 through the refrigerants of the present invention, including each of Refrigerants 1-4, maintains the operating electrical device at or within such preferred temperature ranges. Preferably, the refrigerant 30 of the present invention, which has absorbed heat from the device, is in thermal contact with a heat sink, represented schematically as 40, at a temperature below the temperature of the heat transfer fluid 30 and thereby transfers the heat generated by the device 10 to the heat sink 40. In this way, the heat-depleted refrigerant 50 can be returned to the electronic device 10 to repeat the cycle of cooling.

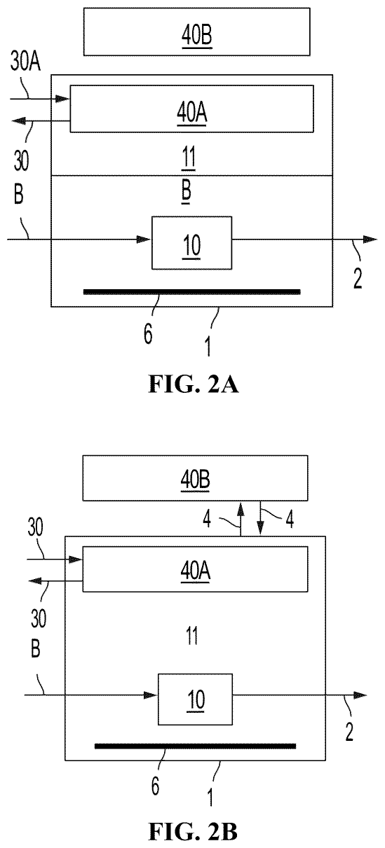

In a preferred embodiment of the present methods, the step of removing heat through the refrigerants of the present invention, including each of Refrigerants 1-4, comprises evaporating the refrigerant using the heat generated by the operation of the electronic device, and the step of transferring that heat from the refrigerant to the heat sink comprises condensing the refrigerant by rejecting heat to the heat sink. In such methods, the temperature of the refrigerants of the present invention, including each of Refrigerants 1-4, during said evaporation step is preferably greater than 50° C., or preferably greater than about 55° C., or preferably in the range of from about 55° C. to about 85° C., or preferably from about 65° C. to about 75° C. Applicants have found that the refrigerants of the present invention, including each of Refrigerants 1-4, provide excellent performance in such methods and at the same time allow the use of relatively low cost, lightweight and reliable equipment to provide the necessary cooling, as will be explained further in connection with particular embodiments as described in connection with FIG. 2A below.

In a further preferred embodiment of the present methods, the step of removing heat through the refrigerants of the present invention, including each of Refrigerants 1-4, comprises adding sensible heat to the refrigerant (e.g., raising the temperature of the liquid up to about 70° C. or less at about atmospheric pressure, i.e., wherein the fluid is not required to be in a high pressure container or vessel) using the heat generated by the operation of the electronic device, and the step of transferring that heat from the refrigerant to a heat sink and thereby reducing the liquid temperature by rejecting heat to the heat sink. The cooled liquid is then returned to thermal contact with the electrical device wherein the cycle starts over. In preferred embodiments, the temperature of the refrigerant that transfers heat to the heat sink is greater than about 40° C., or preferably greater than about 55° C., or preferably in the range of from about 45° C. to about 70° C., or preferably from about 45° C. to about 65° C., and preferably is at a pressure that is about atmospheric. Applicants have found that the refrigerants of the present invention, including each of Refrigerants 1-4, provide excellent performance in such methods and at the same time allow the use for relatively low cost, lightweight and reliable equipment to provide the necessary cooling, as will be explained further in connection with particular embodiments as described in connection with FIG. 2B below.

It will be appreciated by those skilled in the art that the present invention comprises systems and methods which use both sensible heat transfer and phase change heat transfer as described above.

A particular method according to the present invention will now be described in connection with FIGS. 2A and 2B in which an electronic device 10 is contained in an appropriate container 12, and preferably a sealed container, and is in direct contact with, and preferably fully immersed in liquid the refrigerants 11A (shown schematically by gray shading) of the present invention, including each of Refrigerants 1-4. For the purposes of convenience, such cooling methods, devices and systems are sometimes referred to herein as “immersion cooling” methods, devices and systems.

In immersion cooling methods, devices and systems used to cool electrical devices or components, the operating electronic device 10 has a source of electrical energy and/or signals 20 flowing into and/or out of the container 12 and into and/or out of device 10, which generates heat as a result of its operation based on the electrical energy and/or signals 20. As those skilled in the art will appreciate, it is a significant challenge to discover a refrigerant that can perform effectively in such applications since the fluid must not only provide all of the other properties mentioned above, but it must also be able to do so while in intimate contact with an operating electronic device, that is, one which involves the flow of electrical current/signals. It will be appreciated that many fluids that might be otherwise viable for use in such applications will not be useable because they will either short-out the device, degrade when exposed to the conditions created by the operation of the electronic device (i.e., degrade the cooling effect over time and/or the operating stability of the device), or have some other property detrimental to operation when in contact with an operating electronic device.

In contrast, the present thermal management methods produce excellent and unexpected results by providing the refrigerants of the present invention, including each of Refrigerants 1-4, in direct thermal and physical contact with the device 10 as it is operating. This heat of operation is safely and effectively transferred to the refrigerant 11A by: (a) causing the liquid phase of the fluid refrigerant to evaporate and form vapor 11B; or (b) raising the temperature of the liquid refrigerant 11A; or (c) a combination of (a) and (b).

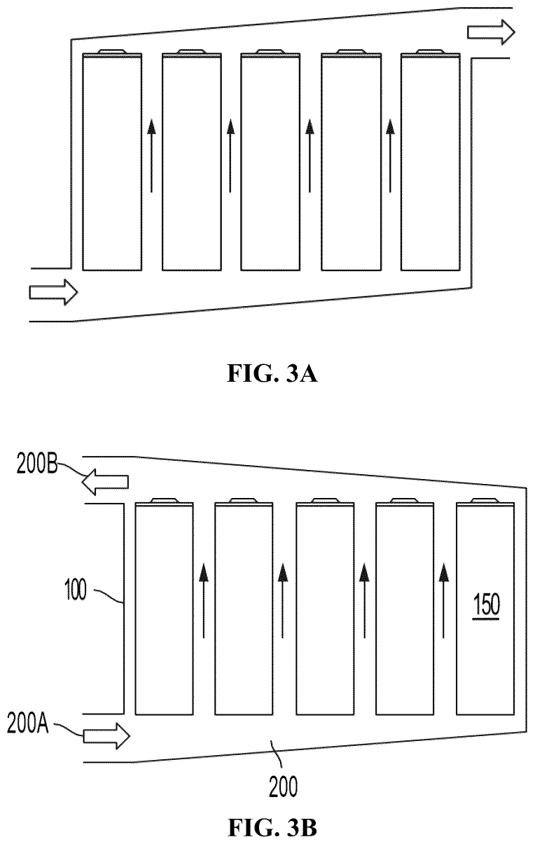

When the refrigerants of the present invention, including each of Refrigerants 1-4, is a single-phase liquid, it will remain liquid when heated by the heat-generating component. Thus, the refrigerant can be brought into contact with the heat generating component, resulting in the removal of the heat from the heat generating component and the production of a refrigerant with a higher temperature. The refrigerant is then transported to a secondary cooling loop, such as a radiator or another refrigerated system. An example of such a system is illustrated in FIGS. 3A and 3B, where the refrigerant enters, at 200A, a battery pack 100 enclosure 200 containing a number of cells 150 and exits, at 200B, the enclosure having taken up heat from the battery pack.

When the refrigerants of the present invention, including each of Refrigerants 1-4, is present in two phases, the heat-generating component is in thermal contact with the refrigerants of the present invention, including each of Refrigerants 1-4, and transfers heat to the refrigerant, resulting in the boiling thereof. The refrigerant is then condensed. An example of such a system is where the heat-generating component is immersed in the refrigerant of the present invention, including each of Refrigerants 1-4, and an external cooling circuit condenses the boiling fluid into a liquid state.

In the case of the phase change heat transfer systems of the present invention, reference is made herein to FIG. 2A. In such an operation, heat is carried away from the device 10 as the refrigerants of the present invention, including each of Refrigerants 1-4, evaporates and the vapor rises through the remaining refrigerant liquid in the container 12. The refrigerant vapor 11B then rejects the heat it has absorbed to a heat sink 40, which can be an enclosed heat sink 40A and/or an external heat sink 40B. An example of a heat sink that is internal to the container 12 are condenser coils 30A and 30B with circulating liquid, such as water, at a temperature below the condensing temperature of the refrigerant vapor. An example of a heat sink that is external to the container 12 would be passing relatively cool ambient air over the container 12 (which preferably in such case include cooling fins or the like), which will serve to condense the heat transfer vapor 11B on the interior surface of the container. As a result of this condensation, liquid refrigerant is returned to the pool of liquid fluid 11A in which the device 10 remains immersed in operation.

In the case of a sensible heat transfer systems of the present invention, reference is made herein to FIG. 2B. In such an operation heat is carried away from the device as the temperature of liquid 11A, including the refrigerants of the present invention, including each of Refrigerants 1-4, increases upon accepting heat being generated by the device, which is immersed, and preferably substantially fully immersed in the refrigerant 11A of the present invention. The higher temperature refrigerant liquid 11A then rejects the heat it has absorbed to a heat sink 40, which can be an enclosed heat sink 40A and/or an external heat sink 40B. An example of a heat sink that is internal to the container 12 are cooling coils 30A and 30B with circulating liquid, such as water, at a temperature below the temperature of heated liquid. An example of a heat sink that is external to the container 12 would be removing heated liquid 11A from the container through a conduit 45 where it is thermally contacted with a cool fluid, such as might be provided by relatively cool ambient air, or cooled water or refrigerant, which will serve to lower the temperature of the liquid. Cooled liquid is then returned via conduit 46.

Optionally, but preferably in certain embodiments involving thermal management of the batteries used in electronic vehicles, the thermal management system includes a heating element which is able to heat the refrigerants of the present invention, including each of Refrigerants 1-4, such as for example an electrical heating element 60 which is also immersed in the refrigerant. As those skilled in the art will appreciate, the batteries in electronic vehicles (which would correspond to the operating electronic device 10 in FIGS. 2A and 2B) can reach relatively low temperatures while parked outside in the winter months in many geographical locations, and frequently such low temperature conditions are not desirable for battery operation. Accordingly, the thermal management system of the present invention can include sensors and control modules (not shown) which turn on the heating element when the battery temperature is below a predetermined level. In such a case, the heater 60 would be activated, the refrigerant liquid 11A would be heated, and would in turn transfer this heat to the electronic device 10 until the minimum temperature is reached. Thereafter during operation, the refrigerants of the present invention, including each of Refrigerants 1-4, would serve the cooling function as described above.

For the purposes of this invention, the refrigerants of the present invention, including each of Refrigerants 1-4, can be in direct contact with the heat-generating component or in indirect contact with the heat-generating component.

When the refrigerants of the present invention, including each of Refrigerants 1-4, is in indirect contact with the heat-generating component, the refrigerant fluid can be used in a closed system in the electronic device, which may include at least two heat exchangers. When the refrigerants of the present invention, including each of Refrigerants 1-4, are used to cool the heat-generating component, heat can be transferred from the component to the refrigerant, usually through a heat exchanger in contact with at least a part of the component or the heat can be transferred to circulating air which can conduct the heat to a heat exchanger that is in thermal contact with the refrigerant.

In a particularly preferred feature of the present invention, the refrigerants of the present invention, including each of Refrigerants 1-4, is in direct contact with the heat-generating component. In particular, the heat generating component is fully or partially immersed in the refrigerant. Preferably the heat generating component is fully immersed in the refrigerants of the present invention, including each of Refrigerants 1-4. The refrigerant, as a warmed fluid or as a vapor, can then be circulated to a heat exchanger which takes the heat from the fluid or vapor and transfers it to the outside environment by way of a heat sink such as ambient air or water cooled by ambient air or otherwise. After this heat transfer, the cooled refrigerant (cooled or condensed) is recycled back into the system to cool the heat-generating component.

Electrical conductivity and/or dielectric strength of a refrigerant becomes important if the fluid comes in direct contact with the electronic components of the electronic device (such as in direct immersion cooling), or if the refrigerant leaks out of a cooling loop or is spilled during maintenance and comes in contact with the electrical circuits. Thus, the refrigerants of the present invention, including each of Refrigerants 1-4, is preferably an electrically insulating thermal management fluid.

The refrigerants of the present invention, including each of Refrigerants 1-4, may be recirculated passively or actively in the device, for example by using mechanical equipment such as a pump. In a preferred feature of the present invention, the refrigerants of the present invention, including each of Refrigerants 1-4, is recirculated passively in the device.

Passive recirculating systems work by transferring heat from the heat-generating component to the refrigerant until it typically is vaporized, allowing the heated vapor to proceed to a heat exchange surface at which it transfers its heat to the heat exchanger surface and condenses back into a liquid. It will be appreciated that the heat exchange surface can be part of a separate heat exchange unit and/or can be integral with the container, as described above for example in connection with FIG. 2. The condensed liquid then returns, preferably fully passively by the force of gravity and/or a wicking structure, into the refrigerant in contact with the heat-generating component. Thus, in a preferred feature of the invention, the step of transferring heat from the heat-generating component to the refrigerants of the present invention, including each of Refrigerants 1-4, causes the thermal management fluid to vaporize.

Examples of passive recirculating systems include a heat pipe or a thermosyphon. Such systems passively recirculate the refrigerants of the present invention, including each of Refrigerants 1-4, using gravity. In such a system, the refrigerant is heated by the heat-generating component, resulting in a heated refrigerant which is less dense and more buoyant. This refrigerant travels to a storage container, such as a tank where it cools and condenses. The cooled refrigerant then flows back to the heat source.

### Electrical Device Cooling

The present invention includes the use of the refrigerants of the present invention, including each of Refrigerants 1-4, to cool and optionally heat electronic devices that produce or include a component that is a heat-generating component. The heat-generating component can be any component that includes an electronic element that generates heat as part of its operation. For the purposes of this invention, the heat generating component includes but is not limited to: semiconductor integrated circuits (ICs), electrochemical cells, power transistors, resistors, and electroluminescent elements, such as microprocessors, wafers used to manufacture semiconductor devices, power control semiconductors, electrical distribution switch gear, power transformers, circuit boards, multi-chip modules, packaged or unpackaged semiconductor devices, semiconductor integrated circuits, fuel cells, lasers (conventional or laser diodes), light emitting diodes (LEDs), and electrochemical cells, e.g. used for high power applications such as, for example, hybrid or electric vehicles.

For the purpose of this invention, the electronic device includes but is not limited to: personal computers, microprocessors, servers, cell phones, tablets, digital home appliances (e.g., televisions, media players, games consoles etc.), personal digital assistants, datacenters, batteries both stationary and in vehicles, including Li-ion batteries and other batteries used in hybrid or electric vehicles, wind turbine, train engine, or generator. Preferably the electronic device is a hybrid or electric vehicle.

The present invention further relates to an electronic device comprising a the refrigerants of the present invention, including each of Refrigerants 1-4. For the purposes of this invention, the refrigerant is provided for cooling and/or heating the electronic device.

The present invention further relates to an electronic device comprising a heat generating component and a refrigerant of the present invention, including each of Refrigerants 1-4, for cooling, and optionally heating, the electronic device.

The present invention further relates to an electronic device comprising a heat generating component, a heat exchanger, a pump and a refrigerant of the present invention, including each of Refrigerants 1-4. For the purpose of this invention, the electronic device can be any such device, including but not limited to personal computers, microprocessors, servers, cell phones, tablets, digital home appliances (e.g. televisions, media players, games consoles etc.), personal digital assistants, datacenters, hybrid or electric vehicles, batteries both stationary and in vehicles, electrical drive motors, fuel cells (e.g., hydrogen fuel cells) and electrical generators, preferably wherein the electronic device is in a hybrid vehicle, or electric vehicle, or wind turbine, or train.

For the purposes of this invention, the heat generating component can be any electrical component that generates heat during operation, but preferably electronic components that generate heat at high levels of heat flux. Examples of heat generating components that can be cooled according to the present invention include semiconductor integrated circuits (ICs), electrochemical cells, power transistors, resistors, and electroluminescent elements, such as microprocessors, wafers used to manufacture semiconductor devices, power control semiconductors, electrical distribution switch gear, power transformers, printed circuit boards (PCBs), multi-chip modules, packaged or unpackaged semiconductor devices, semiconductor integrated circuits, fuel cells, lasers (conventional or laser diodes), light emitting diodes (LEDs), and electrochemical cells, e.g. used for high power applications such as, for example, hybrid or electric vehicles.

### Lithium-Ion Battery Cooling Systems

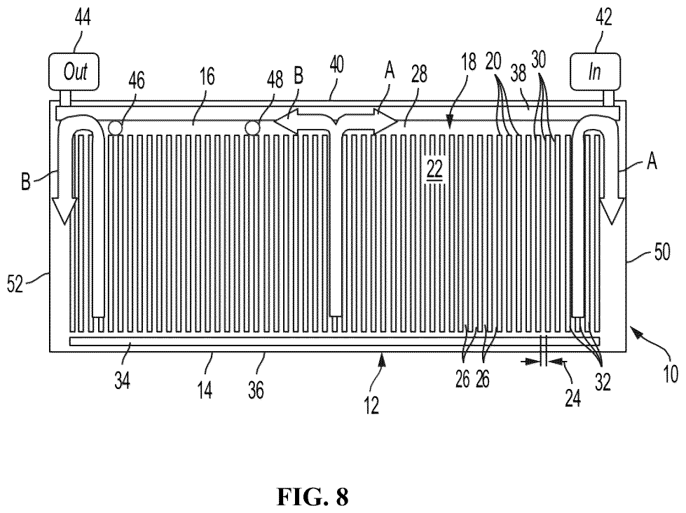

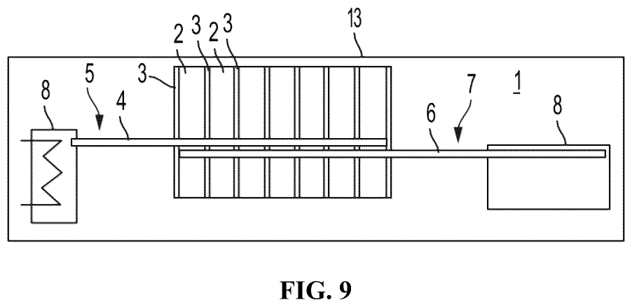

Examples of the present thermal management methods useful for lithium-ion battery cooling, including the use of refrigerants of the present invention, including each of Refrigerants 1-4, in such methods, including Heat Transfer Method 1, will now be described in connection with FIG. 8. A vehicle battery pack with a self-contained liquid cooling system 10 comprising a module 12 formed of a container 14 with an interior space 16 for supporting a battery assembly 18. The container 14 is a closed and sealed container 14 for forming a self-contained liquid cooling system 10. The battery assembly 18 includes a plurality of battery cells 20 such as a plurality of Lithium-ion (Li-ion) batteries for use in a hybrid vehicle. In another embodiment, the plurality of battery cells 20 is Li-ion batteries for use in a Battery Electric Vehicle (BEV). Additional batteries for use with other prime mover vehicles may be provided with the liquid cooling system 10 of the present invention, where each battery cell includes active material for generating power from an electrochemical reaction within the interior space 16 of the container 14. The battery cells 20 are preferably stacked to form a battery cell stack 22. In the embodiment shown, a gap 24 between each battery cell 20 is between 0.25-0.50 mm, forming a fluid channel 26 between each battery cell 20. In another embodiment, the gap 24 may be less than 0.25 mm. It is understood that other gap sizes can be used as desired.

A composition of the present invention, including Compositions 1, and the refrigerants of the present invention, including each of Refrigerants 1-4, is disposed within the interior space 16 of the container 14 and the fluid level shown is such that the battery assembly 18 is completely immersed within the composition/refrigerant of the present invention. The composition of the present invention, including Composition 1, and the refrigerants of the present invention, including each of Refrigerants 1-4, is in contact with the battery cells 20 through the fluid channels 26 formed by gaps 24.

A heating element 34 is located at a base area 36 of the container 14. The heating element 34 shown is an electronic heating element. It is understood that other heating element types may be used. The heating element 34 is shown as a single element; however, multiple heating elements 34 such as heating plates may be provided.

A cooling element 38 is located at an upper area 40 of the container 14. The cooling element 38 may be a chilled water condenser having an inlet 42 and an outlet 44 extending beyond the walls of the sealed container 14 for importing and exporting water for the cooling element 38. In another embodiment, the cooling element 38 may be a chilled water plate. In still another embodiment, the cooling element 38 may be a thin aluminum heat sink having external chilled water travelling through the cooling element 38. The cooling element 38 may be a graphite foil impregnated with an electrically nonconductive polymer. The cooling element may also be formed from copper.

In the embodiment shown, arrows “A” and “B” indicate a flow 28 of the composition of the present invention, including Composition 1, and the refrigerants of the present invention, including each of Refrigerants 1-4. Upon heating of each battery cell 20 by the heating element 34, the fluid 28, including compositions of the present invention, including Compositions 1, and the refrigerants of the present invention, including each of Refrigerants 1-4, is exposed to a front surface area 30 and a rear surface area 32 of the battery cells 20, and will boil. The heated coolant 28 will rise and flow to the top of the battery cell stack 22 to be cooled by the cooling element 38. The cooled coolant 28 will return to the base area 36, generally following either coolant paths “A” or “B.” Where the general location of the coolant 28 at the moment of boiling is located within the fluid channels 26 of the battery cells 20 in the center area and toward a side 50 of the container 14, the coolant 28 will tend to follow flow path “A”. Similarly, if the general location of the dielectric coolant 28 at the moment of boiling is located within the fluid channels 26 of the battery cells 20 in the center area and toward an opposing side 52 of the container 14, the dielectric coolant 28 will tend to follow flow path “B”.

A coolant temperature sensor 46 is located on or near the cooling element 38. In the embodiment shown, the temperature sensor 46 is located within the area of the outlet 44 of the cooling element 38 and measures a temperature of the dielectric coolant 28 of the present invention at a point of exposure to the cooling element. The temperature sensor 46 may be located anywhere within the battery cell stack 22 as desired.

A coolant level sensor 48 is also provided and is located near the upper area 40 of the container 14 to measure the fluid level of the dielectric coolant 28, including compositions of the present invention, including Composition 1, and the refrigerants of the present invention, including each of Refrigerants 1-4, within the container 14, ensuring complete immersion of the battery assembly 18 within the dielectric coolant 28.

### Heat Pipe Cooling and Heating

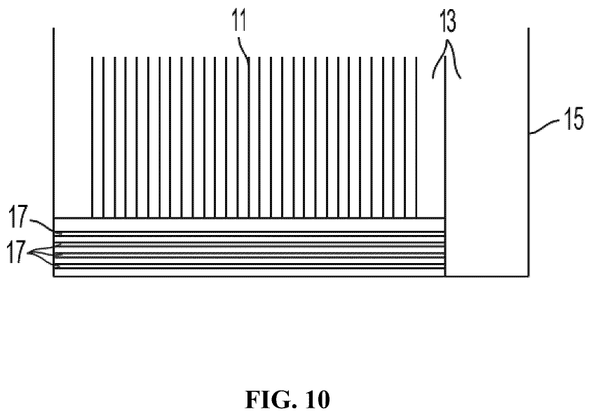

An example of the present heat transfer methods using a heat pipe is now described with respect to FIG. 10, which is a specific example of a heat pipe in an energy storage assembly 1 according to one exemplary embodiment of the invention. The energy storage assembly 1 may be part of a motor vehicle 12, in particular of a hybrid or electric vehicle, and is provided to supply electric power to electric consumers, like e.g., an electrical drive unit (not shown), on the motor vehicle side. The energy storage assembly 1 includes a plurality of electrical energy stores 2. The electrical energy stores 2 are electrically connected via an electric connection element (not shown), in particular in the form of a conductive rail or conductor rail (“bus bar”), i.e., connected in series or in parallel. The electric connection element contacts hereby corresponding electrical connectors (not shown) arranged on respective exposed outer wall sections of corresponding energy storage housings (not shown) of the energy stores 2 in parallel alignment arranged adjacent to one another to thereby form an energy store stack (“stack”). Plate-shaped spacer elements 3 are respectively arranged between the energy stores 2 to separate them and at the same time thermally conductive properties. The spacer elements 3 thus provide, on one hand, spacing between immediately adjacent energy stores 2 so that immediately adjacent energy stores 2 do not contact each other electrically or mechanically. On the other hand, the spacer elements 3 act as a result of their thermally conductive properties as heat conductors for the purpose of cooling the energy stores 2 or the energy storage assembly 1 by dissipating heat, particularly from the contacting energy stores 2, or, for the purpose of heating the energy stores 2 or energy storage assembly 1 by supplying heat, particularly to the contacting energy stores 2. A heat pipe 4 of a first heat pipe assembly 5 and of a heat pipe 6 of a second heat pipe assembly 7 are provide. The heat pipes 4, 6 thus extend along this side surface of the energy store stack and are thermally coupled to the spacer elements 3, respectively. The spacer elements 3 thus form a thermal bridge between the heat pipes 4 of the first heat pipe assembly 5 and the heat pipes 6 of the second heat pipe assembly 7, on one hand, and the energy stores 2, on the other hand. The respective heat pipes 4 of the first heat pipe assembly 5 are arranged and aligned such as to be thermally coupled with respective evaporation zones, in which a contained refrigerant of the present invention, including each of Refrigerants 1-4, can be evaporated, to the spacer elements 3. Heat (evaporation heat) required for the evaporation of the present refrigerant is thus removed from the spacer elements 3 or via the spacer elements 3 from the energy stores 2. The energy stores 2, including the energy storage assembly 1, can thus be cooled via the heat pipes 4 of the first heat pipe assembly 5. Also, the respective condensation zones of the heat pipes 4 of the first heat pipe assembly 5, in which condensation zones a contained gaseous refrigerant of the present invention, including each of Refrigerants 1-4, can be condensed, are thermally coupled with a heat sink 8 in the form of a motor-vehicle-side heat exchanger. Heat (condensation heat) generated during condensation of the present refrigerant can thus be transferred to the heat sink 8. The heat exchanger can be part of the energy storage assembly 1, i.e., belong to or associated with the energy storage assembly 1. The respective heat pipes 6 of the second heat pipe assembly 7 are arranged and aligned such as to be thermally coupled with their respective condensation zones, in which the contained gaseous refrigerant of the present invention can be condensed, to the spacer elements 3. Heat (condensation heat) can thus be transferred during condensation of the present refrigerant to the spacer elements 3 or via the spacer elements 3 to the energy stores 2. Thus, the energy stores 2 and the energy storage assembly 1, can be heated via the heat pipes 6 of the second heat pipe assembly 7. Also, the respective evaporation zones of the heat pipes 6 of the second heat pipe assembly 7, in which evaporation zones a contained refrigerant of the present invention can be evaporated, are thermally coupled with a heat source 9 in the form of a functional component, i.e., e.g., a charger or a control device or a control electronics, associated to the energy storage assembly 1. Heat (evaporation heat) required for the evaporation of the refrigerant can thus be removed from the heat source 9. Thus, the functional component can be cooled via the heat pipes 6 of the second heat pipe assembly 7. The two heat pipe assemblies 5, 7, and their associated heat pipes 4, 6 enables implementation of a temperature control device for controlling the temperature, i.e., for heating or cooling, of the energy stores 2 of the energy storage assembly 1. The heat pipes useful according to the present invention include both gravity return heat pipe, capillary return heat pipe and gravity/capillary return heat pipes.

### Organic Rankine Cycles

When a refrigerant of the present invention, including each of Heat Transfer Compositions A-D, are used in an Organic Rankine cycle, it may be referred to as a working fluid.

The working fluid therefore corresponds to refrigerant as discussed in this application. All preferred features of the heat transfer fluid apply to the working fluid as described herein.

Rankine cycle systems are known to be a simple and reliable means to convert heat energy into mechanical energy in the form of shaft power. In industrial settings, it may be possible to use flammable working fluids such as toluene and pentane, particularly when the industrial setting has large quantities of flammables already on site in processes or storage. However, for instances where the risk associated with use of a flammable and/or toxic working fluid is not acceptable, such as power generation in populous areas or near buildings, it is necessary or at least highly desirable to use non-flammable and/or refrigerants with acceptable toxicity as the working fluid. There is also a drive in the industry for these materials to be environmentally acceptable in terms of GWP.

The process for recovering waste heat in an Organic Rankine cycle according to the present invention preferably involves pumping liquid-phase working fluids of the present invention, including the refrigerant of the present invention, including each of Heat Transfer Compositions A-D, through a boiler where an external (waste) heat source, such as a process stream, heats the working fluid causing it to evaporate into a saturated or superheated vapor. This vapor is expanded through a turbine wherein the waste heat energy is converted into mechanical energy. Subsequently, the vapor phase working fluid is condensed to a liquid and pumped back to the boiler in order to repeat the heat extraction cycle.

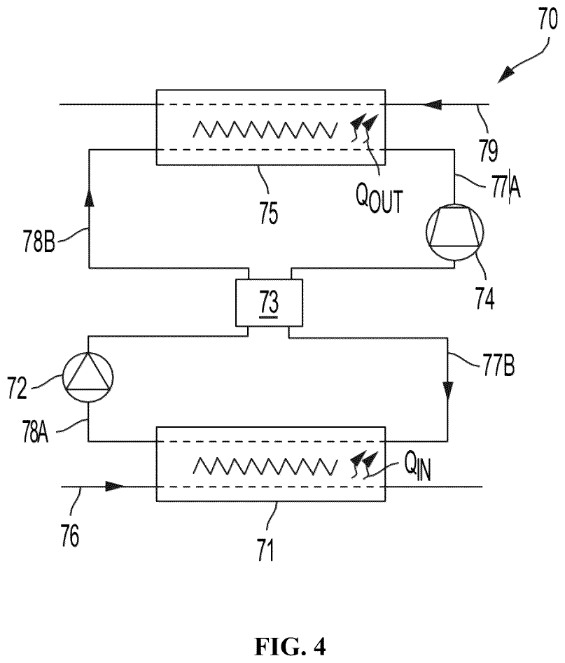

Referring to FIG. 4, in an exemplary organic Rankine cycle system 70, working fluid of the present invention, including refrigerant of the present invention, including each of Heat Transfer Compositions A-D, is circulated between an evaporator 71 and a condenser 75, with a pump 72 and an expansion device 74 functionally disposed therebetween. In the illustrated embodiment, an external flow of fluid is directed to evaporator 71 via external warm conduit 76. External warm conduit 76 may carry fluid from a warm heat source, such as a waste heat source from industrial processes (e.g., power generation), flue gases, exhaust gases, geothermal sources, etc.

Evaporator 71 is preferably configured as a heat exchanger which may include, e.g., a series of thermally connected, but fluidly isolated, tubes carrying fluid from warm conduit 76 and fluid from working fluid conduit 77B respectively. Thus, evaporator 71 facilitates the transfer of heat QIN from the warm fluid arriving from external warm conduit 76 to the relatively cooler (e.g., “cold”) working fluid arriving from expansion device 74 via working fluid conduit 77B.

The working fluid of the present invention, including the present fluoroolefin, thus exits from evaporator 71, having been warmed by the absorption of heat QIN, and then travels through working fluid conduit 78A to pump 72. Pump 72 pressurizes the working fluid, thereby further warming the fluid through external energy inputs (e.g., electricity). The resulting “hot” fluid passes to an input of condenser 75 via conduit 78B, optionally via a regenerator 73 as described below.

Condenser 75 is configured as a heat exchanger similar to evaporator 71, and may include, e.g., a series of thermally connected, but fluidly isolated, tubes carrying fluid from cool conduit 79 and fluid from working fluid conduit 78B respectively. Condenser 75 facilitates the transfer of heat QOUT to the cool fluid arriving from external cool conduit 79 to the relatively warmer (e.g., “hot”) working fluid of the present invention, including the refrigerant of the present invention, including each of Heat Transfer Compositions A-D, arriving from pump 72 via working fluid conduit 78B.

The working fluid of the present invention, including each of Heat Transfer Compositions A-D and RB1-RB28, exiting from condenser 75, having thus been cooled by the loss of heat QOUT, then travels through working fluid conduit 77A to expansion device 74. Expansion device 74 allows the working fluid to expand, thereby further cooling the fluid. At this stage, the fluid of the present invention, including the present fluoroolefins, may perform work, e.g., by driving a turbine. The resulting “cold” fluid passes to an input of evaporator 71 via conduit 77B, optionally via a regenerator 73 as described below, and the cycle begins anew.

Thus, working fluid conduits 77A, 77B, 78A and 78B define a closed loop such that the working fluid contained therein may be reused indefinitely, or until routing maintenance is required.

In the illustrated embodiment, regenerator 73 may be functionally disposed between evaporator 71 and condenser 75. Regenerator 73 allows the “hot” working fluid of the present invention, including the present fluoroolefins, exiting from pump 72 and the “cold” working fluid issued from expansion device 74 to exchange some heat, potentially with a time lag between deposit of heat from the hot working fluid and release of that heat to the cold working fluid. In some applications, this can increase the overall thermal efficiency of Rankine cycle system 70.

The invention also provides a process for converting thermal energy to mechanical energy in a Rankine cycle, the method comprising the steps of i) vaporizing a working fluid of the invention, including refrigerant of the present invention, including each of Heat Transfer Compositions A-D, with a heat source and expanding the resulting vapor, then ii) cooling the working fluid with a heat sink to condense the vapor, wherein the working fluid is a refrigerant of the present invention, including each of Heat Transfer Compositions A-D.

The mechanical work may be transmitted to an electrical device such as a generator to produce electrical power.

The heat source maybe provided by, for example, a thermal energy source selected from industrial waste heat, solar energy, geothermal hot water, low pressure steam, distributed power generation equipment utilizing fuel cells, prime movers, or an internal combustion engine. The low-pressure steam is preferably a low-pressure geothermal steam or is provided by a fossil fuel powered electrical generating power plant.

The heat source is preferably provided by a thermal energy source selected from industrial waste heat, or an internal combustion engine.

It will be appreciated that the heat source temperatures can vary widely, for example from about 90° C. to >800° C., and can be dependent upon a myriad of factors including geography, time of year, etc. for certain combustion gases and some fuel cells.

Systems based on sources such as waste water or low pressure steam from, e.g., a plastics manufacturing plants and/or from chemical or other industrial plant, petroleum refinery, and related word forms, as well as geothermal sources, may have source temperatures that are at or below about 175° C. or at or below about 100° C., and in some cases as low as about 90° C. or even as low as about 80° C. Gaseous sources of heat such as exhaust gas from combustion process or from any heat source where subsequent treatments to remove particulates and/or corrosive species result in low temperatures may also have source temperatures that are at or below 200° C., at or below about 175° C., at or below about 130° C., at or below about 120° C., at or below about 100° C., at or below about 100° C., and in some cases as low as about 90° C. or even as low as about 80° C.

However, it is preferred in some applications that the heat source has a temperature of at least about 200° C., for example of from about 200° C. to about 400° C.

In an alternative preferred embodiment, the heat source has a temperature of from 400 to 800° C., more preferably 400 to 600° C.

### Heat Pumps

The refrigerant of the present invention, including each of Refrigerants 1-4 and RB1-RB28, may be used in a high temperature heat pump system.

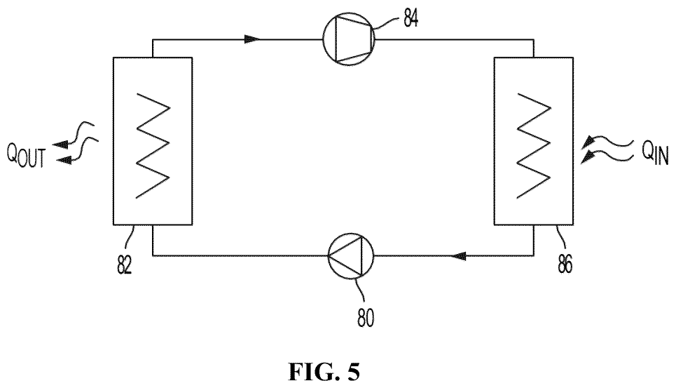

Referring to FIG. 5, in one exemplary heat pump system, compressor 80, such as a rotary, piston, screw, or scroll compressor, compresses a refrigerant of the present invention, including each of Heat Transfer Compositions A-D, which is conveyed to a condenser 82 to release heat QOUT to a first location, followed by passing the refrigerant through an expansion device 84 to lower the refrigerant pressure, followed by passing the refrigerant through an evaporator 86 to absorb heat QIN from a second location. The refrigerant is then conveyed back to the compressor 80 for compression.

The present invention provides a method of heating a fluid or body using a high temperature heat pump, said method comprising the steps of (a) condensing a refrigerant of the present invention, including each of Heat Transfer Compositions A-D, in the vicinity of the fluid of body or be heated, and (b) evaporating said refrigerant.

Examples of high temperature heat pumps include a heat pump tumble dryer or an industrial heat pump. It will be appreciated the heat pump may comprise a suction line/liquid line heat exchanger (SL-LL HX). By “high temperature heat pump”, it is meant a heat pump that is able to generate temperatures of at least about 80° C., preferably at least about 90° C., preferably at least about 100° C., more preferably at least about 110° C.

### Secondary Loop System

The refrigerant of the present invention, including each of Refrigerants 1-4 or Heat Transfer Compositions A-D and RB1-RB28, may be used as secondary refrigerant fluid in a secondary loop system.

A secondary loop system contains a primary vapor compression system loop that uses a primary refrigerant and whose evaporator cools the secondary loop fluid. The secondary refrigerant fluid, including refrigerant of the present invention, including each of Heat Transfer Compositions A-D, then provides the necessary cooling for an application. The secondary refrigerant fluid should preferably have acceptable toxicity since the fluid in such a loop is potentially exposed to humans in the vicinity of the cooled space. In other words, the refrigerant of the present invention, including each of Heat Transfer Compositions A-D, may be used as a “secondary refrigerant fluid” in a secondary loop system.

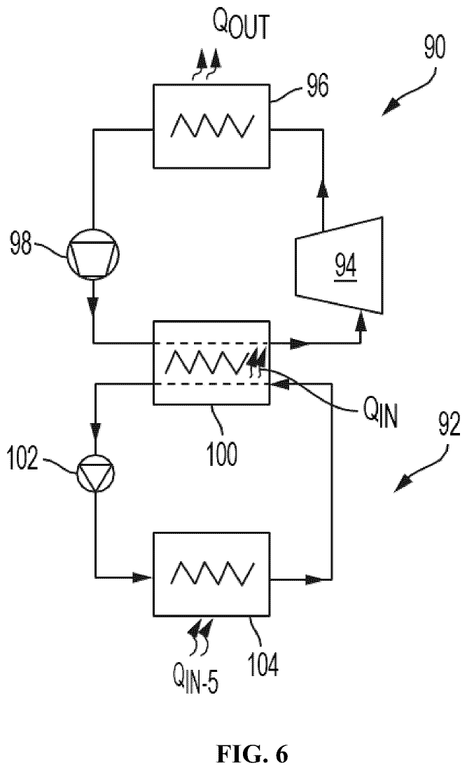

Referring to FIG. 6, one exemplary secondary loop system includes a primary loop 90 and a secondary loop 92. In primary loop 90, compressor 94, such as a rotary, piston, screw, or scroll compressor, compresses a primary refrigerant, which is conveyed to a condenser 96 to release heat QOUT to a first location, followed by passing the primary refrigerant through an expansion device 98 to lower the refrigerant pressure, followed by passing the primary refrigerant through a refrigerant/secondary fluid heat exchanger 100 to exchange heat QIN with a secondary fluid, including the present fluoroolefins, with the secondary fluid pumped through secondary loop 92 via a pump 102 to a secondary loop heat exchanger 104 to exchange heat with a further location, for example to absorb heat QIN-S to providing cooling to the further location.

The primary fluid used in the primary loop (vapor compression cycle, external/outdoors part of the loop) may be selected from but not limited to HFO-1234ze(E), HFO-1234yf, propane, R455A, R32, R466A, R44B, R290, R717, R452B, R448A, and R449A, preferably HFO-1234ze(E), HFO-1234yf, or propane.

The secondary loop system may be used in refrigeration or air conditioning applications, that is, the secondary loop system may be a secondary loop refrigeration system or a secondary loop air conditioning system.

Examples of refrigeration systems which can include a secondary loop refrigeration system that include a secondary refrigerant of the present invention, including the present fluoroolefins, include: a low temperature refrigeration system, a medium temperature refrigeration system, a commercial refrigerator, a commercial freezer, an industrial freezer, an industrial refrigerator, and a chiller.

Examples of air conditioning systems which can include a secondary loop air conditioning system which utilize a refrigerant of the present invention, including the present fluoroolefins, include in mobile air conditioning systems or stationary air conditioning systems. Mobile air-conditioning systems including air conditioning of road vehicles such as automobiles, trucks and buses, as well as air conditioning of boats, and trains. For example, where a vehicle contains a battery or electric power source.

Examples of stationary air conditioning systems which can include a secondary loop air conditioning system which utilize a refrigerant of the present invention, including the present fluoroolefins, include: a chiller, particularly a positive displacement chiller, more particularly an air cooled or water-cooled direct expansion chiller, which is either modular or conventionally singularly packaged, a residential air conditioning system, particularly a ducted split or a ductless split air conditioning system, a residential heat pump, a residential air to water heat pump/hydronic system, an industrial air conditioning system, a commercial air conditioning system, particularly a packaged rooftop unit and a variable refrigerant flow (VRF) system, and a commercial air source, water source or ground source heat pump system.

A particularly preferred heat transfer system according to the present invention is an automotive air conditioning system comprising a vapor compression system (the primary loop) and a secondary loop air conditioning system, wherein the primary loop contains HFO-1234yf as the refrigerant and the second loop contains a refrigerant or heat transfer composition of the present invention, including the present fluoroolefins. In particular, the secondary loop can be used to cool a component in the car engine, such as the battery.

It will be appreciated that the secondary loop air conditioning or refrigeration system may comprise a suction line/liquid line heat exchanger (SL-LL HX).

The present heat transfer fluids, or heat transfer compositions which can include a secondary loop air conditioning system which utilize a refrigerant of the present invention, including Heat Transfer Compositions A-D and RB1-RB28, may be used as a replacement for existing fluids.

The invention includes a method of replacing an existing heat transfer fluid in a heat transfer system, said method comprising the steps of (a) removing at least a portion of said existing heat transfer fluid from said system, and subsequently (b) introducing into said system a refrigerant of the present invention, including Heat Transfer Compositions A-D. Step (a) may involve removing at least about 5 wt. %, at least about 10 wt. %, at least about 15 wt. %, at least about 50 wt. % at least about 70 wt. %, at least about 90 wt. %, at least about 95 wt. %, at least about 99 wt. % or at least about 99.5 wt. % or substantially all of said existing heat transfer fluid from said system prior to step (b).

The method may optionally comprise the step of flushing said system with a solvent after conducting step (a) and prior to conducting step (b).

For the purposes of this invention, the refrigerant of the present invention, including each of Heat Transfer Compositions A-D, can be used to replace an existing fluid in an electronic device, in an Organic Rankine cycle, in a high temperature heat pump or in a secondary loop.

For example, the refrigerant of the present invention, including each of Heat Transfer Compositions A-D, may be used as a replacement for existing fluids such as HFC-4310mee, HFE-7100 and HFE-7200. Alternatively, the refrigerant of the present invention, including each of Refrigerants 1-4 and RB1-RB28, can be used to replace water and glycol. The replacement may be in existing systems, or in new systems which are designed to work with an existing fluid. Alternatively, the refrigerant of the present invention, including each of Heat Transfer Compositions A-D, can be used in applications in which the existing refrigerant was previously used. Alternatively, the refrigerant of the present invention, including each of Heat Transfer Compositions A-D, may be used to retrofit an existing refrigerant in an existing system. Alternatively, the refrigerant of the present invention, including each of Heat Transfer Compositions A-D, may be used in new systems which are designed to work with an existing refrigerant.

The invention provides a method of replacing an existing refrigerant in a heat transfer system, said method comprising the steps of (a) removing at least a portion of said existing refrigerant from said system, and subsequently (b) introducing into said system refrigerant of the present invention, including each of Heat Transfer Compositions A-D. The existing refrigerants may be selected, for example, from HFC-4310mee, HFE-7100 and HFE-7200.

Step (a) may involve removing at least about 5 wt. %, at least about 10 wt. %, at least about 15 wt. %, at least about 50 wt. % at least about 70 wt. %, at least about 90 wt. %, at least about 95 wt. %, at least about 99 wt. % or at least about 99.5 wt. % of said existing refrigerant from said system prior to step (b).

The method may optionally comprise the step of flushing said system with a solvent after conducting step (a) and prior to conducting step (b).

### III.B. Heating and Cooling During Manufacture

With particular reference to FIG. 11, a side cross-sectional view of a refrigerant cooled conventional wet-etching station is illustrated. The basic operation of such a wet-etching station includes providing a container 15 which holds a bath of chemical etchant 13 for etching a plurality of semiconductor wafers 11. The temperature of the chemical etchant 13 is preferably kept as uniform as possible with the goal of maintaining the surface of wafers 11 such that they are uniformly etched by being immersed in the bath 15 of chemical etchant. To help maintain such a uniform temperature, a plurality of cooling lines 17 are installed in a portion of the container 15 holding chemical etchant bath 13 such that the cooling lines 17 are in contact with the chemical etchant 13. During wet etching process, the present refrigerants, including particularly each of Refrigerants 1-4, circulate through the coolant lines 17 and transport heat from the chemical etchant, either by sensible temperature change, or phase change and/or a combination of by sensible temperature change and phase change.

### EXAMPLES

### Example 1-Synthesis of 1,3,4,4,5,5,5-heptafluoro-3-(trifluoromethyl)pent-1-ene (secBuFO)

### Step 1. Synthesis of 1,1,1,2,2,3,5-heptafluoro-5-iodo-3-(trifluoromethyl)pentane

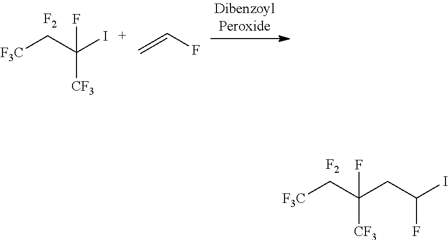

In a clean and dry 600 mL Parr Autoclave with a liquid stirrer, 1.3 g (0.53 mmol) of dibenzoyl peroxide was added. The autoclave was closed and pressurized with high-pressure nitrogen to 700 psi for pressure leak test. After the pressure leak test, the autoclave was pulse-purged alternating with a vacuum pump and low-pressure nitrogen to remove any trace of O₂, water, or volatiles and create a negative pressure inside the autoclave. Then, 515 g of 2-iodononafluorobutane (1.5 mol, 1.2 eq) were transferred to the autoclave through a prefilled cylinder via gravity (upside down cylinder). The mechanical agitator was set up to 1000 RPM while the autoclave was heated to 80° C. Once the autoclave temperature was at 80° C., a constant slow feed of vinyl fluoride (˜1 g/min) was loaded to the autoclave at constant pressure until the total amount of vinyl fluoride added not surpassed 57 g (1.2 mol, 1 eq). Inner temperature constantly and slowly increased (˜0.8° C./min) up to ˜120° C. (proof that reaction is occurring). After all vinyl fluoride was added the system was closed and kept at 80° C. for 5 h (inner pressure drastically dropped after finishing the addition of vinyl fluoride to reach a constant value over ˜1 h). After 5 h, the autoclave was cooled down to room temperature; and then, the headspace was vented via a PFA tubing ending at the back of the fumehood before opening. Once the autoclave was open, the crude was filtered and transferred to a labeled bottle, and GCMS was performed to identify the major products, i.e. 1,1,1,2,2,3,5-heptafluoro-5-iodo-3-(trifluoromethyl)pentane (88% GC Area) and 2-iodononafluorobutane (9% GC Area). To isolate the desired intermediate, the crude was transferred to a 1 L round bottom flask with a magnetic stirrer. The 1 L round bottom flask was attached to a distillation head provided with a cold finger (cooled down to −10° C.) and 2-iodononafluorobutane was distilled out from the crude (B.P. 64-67° C.). After all 2-iodononafluorobutane was removed from the crude, a GC of the reboiler (388 g, 80% yield) was performed to confirm the purity of the desired product (95% pure). Molecular structure was corroborated by ¹H NMR, ¹³C NMR, and ¹⁹F NMR.

### Step 2. Synthesis of 1,3,4,4,5,5,5-heptafluoro-3-(trifluoromethyl)pent-1-ene

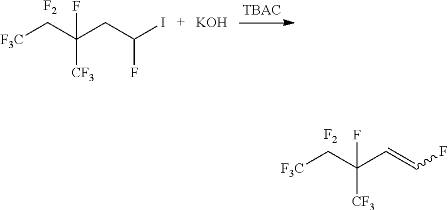

In a clean three neck round bottom flask with a magnetic stirrer, KOH (167 g, 3 mol, 3 eq), TBAC (28 g, 0.1 mol, 0.1 eq) and 500 mL of water were added. A dropping funnel and a distilling head (with a water jacket and a collecting flask) were attached to the three neck round bottom flask. The reaction mixture was stirred and heated up until internal temperature reached 70° C.; and 1,1,1,2,2,3,5-heptafluoro-5-iodo-3-(trifluoromethyl) pentane from step 1 (388 g, 1 mol, 1 eq) was poured to the closed dripping funnel. At a constant drop flow 1,1,1,2,2,3,5-heptafluoro-5-iodo-3-(trifluoromethyl) pentane was added to the reaction mixture; the desired isomeric mixture formed at the reaction temperature evaporates from the reaction mixture and was condensed in the distilling head. After the complete addition of 1,1,1,2,2,3,5-heptafluoro-5-iodo-3-(trifluoromethyl) pentane the reaction mixture was continued heated and stirred for 4 h (at this point no more product condensing was observed in the collecting flask). The reaction mixture was cooled down to room temperature and the isolated isomeric mixture in the collecting flask (216 g) was submitted to GCMS to identify the isomeric mixture. Fraction distillation was performed to isolate the desired isomers; trans-1,3,4,4,5,5,5-heptafluoro-3-(trifluoromethyl)pent-1-ene (164 g, 76% yield) and cis-1,3,4,4,5,5,5-heptafluoro-3-(trifluoromethyl)pent-1-ene (30 g, 14% yield). Molecular structures were corroborated by 1H NMR, 13C NMR, and 19F NMR. After isolation of the trans-1,3,4,4,5,5,5-heptafluoro-3-(trifluoromethyl)pent-1-ene by distillation column, the following impurities were observed at different purity levels for the trans isomer of sec BuFO:

| | trans- | trans- | trans- | trans- | trans- |
|---|---|---|---|---|---|
| Component | secBuFO | secBuFO | secBuFO | secBuFO | secBuFO |
| Name | 98.43% | 99.50% | 99.90% | 99.94% | 99.95% |
| cis- | 0.564 | 0.170 | 0.022 | 0.008 | 0.003 |
| secBUFO | | | | | |
| C₆H₂F₈ | 0.076 | 0.041 | 0.042 | 0.041 | 0.040 |
| Isomer | | | | | |
| CF₃CF₂CF(CF₃)I | 0.015 | 0.011 | 0.001 | 0.011 | 0.007 |

Additionally, the following impurities are observed at different purity levels for the cis isomer of sec BuFO:

| | | | | | trans- |
|---|---|---|---|---|---|
| Component | cis-secBuFO | cis-secBuFO | cis-secBuFO | tcis-secBuFO | secBuFO |
| Name | 98.43% | 99.50% | 99.90% | 99.94% | 99.95% |
| trans- | 0.564 | 0.170 | 0.022 | 0.008 | 0.003 |
| secBUFO | | | | | |
| Other | 1.006 | 0.033 | 0.078 | 0.052 | 0.047 |
| impurities | | | | | |

### Example 2—Synthesis of 1,4,4,4-tetrafluoro-3,3-bis(trifluoromethyl)but-1-ene) (tBuFO)

### Step 1. Synthesis of 1,1-dibromo-3,3,3-trifluoro-2-(trifluoromethyl)prop-1-ene

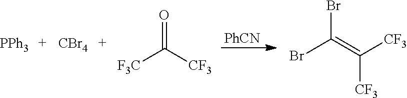 [SMILES: "PhCN" ; "CBr4" ; O=C(C(F)(F)F)C(F)(F)F ; FC(F)(F)C(=C(Br)Br)C(F)(F)F ; "PPh3"]

Synthetic procedure is a hybrid method from J. Am. Chem. Soc., 1993, 115, 5430-5439 and Angew. Chem. Int. Ed. 2021, 60, 27318-27323.

A three neck 1 L flask equipped with a mechanical stirrer, a solid addition funnel, and a condenser further attached to a N2 tee was charged with triphenyl phosphine (39.7 g, 0.2 mol, 2.0 eq) and 200 mL of benzonitrile. The mixture was stirred and cooled down to 10° C. with an ice water bath, and then CBr4 (26.2 g, 0.1 mol, 1.0 eq) was added over 10-min period from the solid addition funnel. After stirring for 1 h at 10° C., the condenser was charged with dry ice/acetone to be cooled down to −78° C. and the reaction mixture was immersed in a dry ice/acetone bath too. Hexafluoroacetone (19.9 g, 0.12 mol, 1.2 eq) was condensed into the solution over a 45-min period. The viscous mixture was stirred for 4 h at room temperature. After stirring for 4 h at room temperature, the mixture was filtered through a pad of CELITE® and washed with benzonitrile (10 mL). The crude was distilled at 85° C./15 torr; and then re-distilled at 45° C./50 torr, collecting the desired product as a colorless liquid (14.07 g, 44% yield). Molecular structure was corroborated by ¹H NMR, ¹³C NMR, ¹⁹F NMR and GCMS.

### Step 2. Synthesis of 4-chloro-1,1,1,4-tetrafluoro-2,2-bis(trifluoromethyl)butane

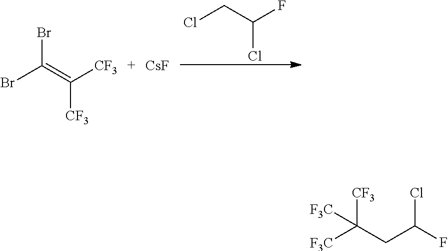

Synthetic procedure from Angew. Chem. Int. Ed. 2021, 60, 27318-27323 and J. Am. Chem. Soc. 2022, 144, 22281-22288.

To a 250 mL PTFE bottle, CsF (114 mmol, 17.3 g, 3.8 eq), 1,1-dibromo-3,3,3-trifluoro-2-(trifluoromethyl)prop-1-ene (36 mmol, 11.6 g, 1.2 eq) and anhydrous DMF (150 mL) are added in glove box. The solution is stirred at room temperature for 30 min. Then, 1,2-dichlorofluoroethane (30 mmol, 3.5 g, 1 eq) is added to this solution. The solution is stirred at room temperature for 3 h. After completion of the reaction, the mixture is quenched by saturated NH4Cl solution, then the mixture is extracted with hexanes three times. The organic phase is dried over MgSO4 and the solvent is reduced under reduced pressure to obtain 7.2 g (80% yield). Molecular structure is corroborated by ¹H NMR, ¹³C NMR, ¹⁹F NMR and GCMS.

### Step 3. Synthesis of 1,4,4,4-tetrafluoro-3,3-bis(trifluoromethyl)but-1-ene

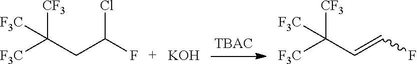

In a clean three neck round bottom flask with a magnetic stirrer, KOH (3.1 g, 55.1 mmol, 3 eq), TBAC (20.7 g, 0.07 mol, 0.1 eq) and 500 mL of water are added. A dropping funnel and a distilling head (with a water jacket and a collecting flask) are attached to the three neck round bottom flask. The reaction mixture is stirred and heated up until internal temperature reached 70° C., and 4-chloro-1,1,1,4-tetrafluoro-2,2-bis(trifluoromethyl)butane from step 2 (7.2 g, 18.3 mmol 1 eq) is poured to the closed dripping funnel. At a constant drop flow 4-chloro-1,1,1,4-tetrafluoro-2,2-bis(trifluoromethyl)butane is added to the reaction mixture; the desired isomeric mixture formed at the reaction temperature evaporates from the reaction mixture and is condensed in the distilling head. After complete addition of 4-chloro-1,1,1,4-tetrafluoro-2,2-bis(trifluoromethyl)butane the reaction mixture is continued heated and stirred for 4 h (at this point no more product condensing was observed in the collecting flask). The reaction mixture is cooled down to room temperature and the isolated isomeric mixture in the collecting flask (2.91 g) is submitted to GCMS to identify the isomeric mixture. Fraction distillation is performed to isolate the desired isomers; trans-1,4,4,4-tetrafluoro-3,3-bis(trifluoromethyl)but-1-ene (2.3 g, 48% yield), and cis-1,4,4,4-tetrafluoro-3,3-bis(trifluoromethyl)but-1-ene (0.77 g, 16%). Molecular structures are corroborated by ¹H NMR, ¹³C NMR, and ¹⁹F NMR.

The following impurities are observed at different purity levels for the trans isomer of tBuFO:

| Component | trans-tBuFO | trans-tBuFO | trans-tBuFO | trans-tBuFO | trans-tBuFO |
|---|---|---|---|---|---|
| Name | 98.43% | 99.50% | 99.90% | 99.94% | 99.95% |
| cis-tBUFO | 0.564 | 0.170 | 0.022 | 0.008 | 0.003 |
| Other | 1.006 | 0.033 | 0.078 | 0.052 | 0.047 |
| impurities | | | | | |

Additionally, the following impurities are observed at different purity levels for the cis isomer of tBuFO:

| Component | cis-tBuFO | cis-tBuFO | cis-tBuFO | cis-tBuFO | cis-tBuFO |
|---|---|---|---|---|---|
| Name | 98.43% | 99.50% | 99.90% | 99.94% | 99.95% |
| trans-tBUFO | 0.564 | 0.170 | 0.022 | 0.008 | 0.003 |
| Other | 1.006 | 0.033 | 0.078 | 0.052 | 0.047 |
| impurities | | | | | |

### Example 3—Physical Property Measurement

The boiling point (“BP”) and dielectric constant (“Dk”) of trans- and cis-secBuFO and trans- and cis-tBuFO were determined and the results are reported below in Table E1. The dielectric property of each of trans- and cis-secBuFO and trans- and cis-tBuFO was determined using the Agilent 85070 Dielectric Probe. All measurements were made at ambient pressure and room temperature (approximately 23° C.). Prior to making measurements, the system was calibrated from 1 GHz to 20 GHz with an open circuit, a short circuit, and DI water (@22.4° C.) standard. The result of the calibration is shown in the Table E1 below for DI H2O, and is consistent with DI water at 22.4° C. The accuracy of the probe is given as: Dielectric constant, er′=er′+/−0.05|er*| with er″=er″+/−0.05|er*| and (loss=er″/er′).

TABLE E1

| Compound | BP, ° C. | Calculated Dk at 20 KHz | GWP | ODP |
|---|---|---|---|---|
| trans-secBuFO | 55.5-55.9 | 2.11 | <50 | 0 |
| cis-secBuFO | 79.27 | 6.48 | <50 | 0 |
| trans-tBuFO | 57.9 | 2.15 | <50 | 0 |
| cis-tBuFO | 76.19 | 6.51 | <50 | 0 |

### Example 4—Battery Cooling in Electric Vehicles Using Each of Refrigerants 1-4

Batteries of electric vehicles develop heat during operation when charging and discharging. The typical design of vehicle batteries differs between three types: cylindrical cells, pouch cells and prismatic cells. All three types have different considerations in terms of heat transfer due to their shape. Prismatic and pouch cells are often used with cooling plates due to the straight outer faces. Cylindrical cells employ cooling ribbons that are in thermal contact with the outer shell of the cells. Extensive heat generation during charging and discharging of the cells can lead to an increase in temperature that can cause decreasing performance and reduced battery lifetime.

A battery cooling plate set up may be used to provide active cooling to a battery and remove the heat (e.g., to remove heat from the battery of an electric vehicle). In this Example, the performance of refrigerant fluids of the present invention, including each of Refrigerants 1-8, compared to 3M Novec 7200 are analyzed for the ability to provide cooling in single phase heat transfer.

It will be appreciated that the convective heat transfer can occur either by direct contact, i.e., when the battery is immersed in the fluid that may be pumped through the battery enclosure or indirectly, i.e., by using a cooling plate with a combination of convective and conductive heat transfer.

The present example uses a round tube with an internal diameter of 0.55 inches to provide a cooling load of 10246 BTU/h (3 kW). The tube length was 30 ft (9.14 m) with an assumed pressure drop of 2.9 PSI (20 kPa). The fluid temperature was 7.2 C (45 F). The internal heat transfer coefficient is determined for turbulent flow. The necessary mass flow rate to remove the cooling load is determined for both fluids. The results of the comparison are shown in the Table E2 below. It can be seen in the results that the necessary mass flow rate to remove the generated heat is about or less than for 3M Novec 7200 and that the useful output (i.e., the heat transfer coefficient) is about or higher than 3M Novec 7200.

TABLE E2

| | | | Internal heat |
|---|---|---|---|
| | Mass Flow | Prandtl | transfer coefficient |
| Fluid | Rate (lb./s) | Number [—] | (BTU/(h-ft2-F) |
| Refrigerants 1-4 | 0.9-1 | 9-11 | 300-350 |
| 3M Novec 7200 | 0.98 | 10.4 | 303.4 |

### Example 5A—Singe Phase (Sensible Heat) Immersion Cooling in Battery Applications Using Each of Refrigerants 1-4

Batteries of electric vehicles develop heat during operation when charging and discharging. The typical design of vehicle batteries differs between three types: cylindrical cells, pouch cells and prismatic cells. All three types have different considerations in terms of heat transfer due to their shape. Extensive heat generation during charging and discharging of the cells can lead to an increase in temperature that can cause decreasing performance and reduced battery lifetime.

Refrigerants 1-4 preferably have low dielectric constants and high dielectric strength, and are non-flammable fluids which allow for direct cooling of the battery cells that are immersed in each of Refrigerants 1-4.

The present example considers a battery module that consists of 1792 cylindrical battery cells of 18650 type. In one case the battery module is cooled by a 50/50 mixture of water/glycol in a flat tube heat exchanger that is on contact with the battery cells. In the other case the cells are immersed in each of Refrigerants 1-4, i.e., are in direct contact with the fluid. The waste heat for the battery module is 8750 W that is evenly distributed over the total number of cells. The assumptions and operating conditions are listed in Table E3A1 and Table E3A2.

TABLE E3A1

| | | | Refrigerants |
|---|---|---|---|
| Parameter | Unit | Water/Glycol | 1-4 |
| Battery diameter | [mm] | 18.5 | 18.5 |
| Battery gap | [mm] | 3.8 | 1.5 |
| Battery height | [mm] | 65 | |
| Number of batteries | [—] | 1792 | |
| Battery mass | [g] | 49 | |
| Battery specific heat | [J/kgK] | 830 | |
| Total battery module | [W] | 8750 | |
| waste heat | | | |
| Fluid flow rate | [kg/s] | 0.1 | |
| Initial module temperature | [° C.] | 30 | |
| Fluid inlet temperature | [° C.] | 10 | |
| Cooling channel height | [mm] | 30 | n/a |
| Cooling channel width | [mm] | 2.8 | n/a |
| Heat exchanger flat tube | [mm] | 0.5 | n/a |
| wall thickness | | | |
| Heat exchanger flat tube | [W/mK] | 3 | n/a |
| thermal conductivity | | | |
| Heat exchanger flat tube | [—] | 0.0003 | n/a |
| relative surface roughness | | | |

TABLE E3A2

| | Minimum cell temperature [° C.] | | Maximum cell temperatures [° C.] | |
|---|---|---|---|---|
| | Water/Glycol | Refrigerants | Water/Glycol | Refrigerants |
| Time | 50/50 | 1-4 | 50/50 | 1-4 |
| 0 | 30.0 | 30.0 | 30.0 | 30.0 |
| 100 | 35.8 | 10-40 | 36.8 | 30-40 |
| 200 | 40.3 | 10-40 | 42.0 | 30-40 |
| 300 | 43.6 | 10-45 | 46.0 | 30-45 |
| 400 | 46.1 | 10-45 | 49.2 | 30-50 |
| 500 | 48.0 | 10-45 | 51.7 | 30-50 |
| 600 | 49.5 | 10-50 | 53.6 | 30-55 |
| 700 | 50.5 | 10-50 | 55.1 | 30-55 |
| 800 | 51.4 | 10-50 | 56.3 | 30-55 |
| 900 | 52.0 | 10-50 | 57.2 | 30-55 |

### Example 5B—Single Phase (Sensible Heat) Data Center Cooling Using Each of Refrigerants 1-4

Data centers, also described herein as server banks or server hubs, are designed to maximize computing and storage capacity while minimizing space requirements. This results in densely packed arrays of servers and networking gear which can lead to concentrated heat generation. In addition, data centers operate around the clock, further contributing to heat build-up. With effective cooling, the efficiency and longevity of server hardware can be improved.

Refrigerants 1-4 preferably have low dielectric constants and high dielectric strength, and are non-flammable fluids, which allow for direct cooling of the data centers that are immersed in each of Refrigerants 1-4, including by causing sensible heat transfer to the refrigerant (i.e., refrigerant temperature change not associated with phase change).

A data center is thus cooled using separately each of Refrigerants 1-4, and the system operates effectively, efficiently, safely and reliably. The electronic components are kept in the most desired operating temperature range while the data center is performing its functions.

### Example 5C—Single Phase (Sensible Heat) Semiconductor Integrated Circuit Cooling Using Each of Refrigerants 1-4

Example 5B is repeated, except the cooling is applied to one or more semiconductor integrated circuit(s) using each of Refrigerants 1-4. Effective and advantageous cooling is achieved by direct contact comprising at least partial immersion of the semiconductor integrated circuit in each of Refrigerants 1-4. The semiconductor integrated circuit(s) are cooled with each of Refrigerants 1-4 and the semiconductor integrated circuit(s) operate effectively, efficiently, safely and reliably while at least partially immersed in Refrigerants 1-4, and the semiconductor integrated circuit(s) are kept in the most desired operating temperature range while performing its functions.

### Example 5D—Single Phase (Sensible Heat) Microprocessor Cooling Using Each of Refrigerants 1-4

Example 5B is repeated, except the cooling is applied to one or more microprocessor(s) using each of Refrigerants 1-4. Effective and advantageous cooling is achieved by direct contact comprising at least partial immersion of the microprocessor(s) in each of Refrigerants 1-4. The microprocessor(s) are cooled with each of Refrigerants 1-4 and the component/article/device operates effectively, efficiently, safely and reliably while at least partially immersed in Refrigerants 1-4, and the microprocessor(s) are kept in the most desired operating temperature range while performing its functions.

### Example 5E—Single Phase (Sensible Heat) Electrochemical Cell Cooling Using Each of Refrigerants 1-4

Example 5B is repeated, except the cooling is applied to one or more electrochemical cell(s) using each of Refrigerants 1-4. Effective and advantageous cooling is achieved by direct contact comprising at least partial immersion of the electrochemical cell(s) in each of Refrigerants 1-4. The electrochemical cell(s) are cooled with each of Refrigerants 1-4 absorbing heat via and the electrochemical cell(s) operate effectively, efficiently, safely and reliably while at least partially immersed in each of Refrigerants 1-4, and the electrochemical cell(s) are kept in the most desired operating temperature range while performing its functions.

### Example 5F—Single Phase (Sensible Heat) Fuel Cell Cooling Using Each of Refrigerants 1-4

Example 5B is repeated, except the cooling is applied to one or more fuel cell(s) using each of Refrigerants 1-4. Effective and advantageous cooling is achieved by direct contact comprising at least partial immersion of the fuel cell(s) in each of Refrigerants 1-4. The fuel cell(s) are cooled with each of Refrigerants 1-4 and the fuel cell(s) operate effectively, efficiently, safely and reliably while at least partially immersed in each of Refrigerants 1-4, and the fuel cell(s) are kept in the most desired operating temperature range while performing its functions.

### Example 5G—Single Phase (Sensible Heat) Resistor Cooling Using Each of Refrigerants 1-4

Example 5B is repeated, except the cooling is applied to one or more resistor(s) using each of Refrigerants 1-4. Effective and advantageous cooling is achieved by direct contact comprising at least partial immersion of the resistor(s) in each of Refrigerants 1-4. The resistor(s) are cooled with each of Refrigerants 1-4 and the resistor(s) operate effectively, efficiently, safely and reliably while at least partially immersed in each of Refrigerants 1-4, and the resistor(s) are kept in the most desired operating temperature range while performing its functions.

### Example 5H—Single Phase (Sensible Heat) Power Transistor Cooling Using Each of Refrigerants 1-4

Example 5B is repeated, except the cooling is applied to one or more power transistor(s) using each of Refrigerants 1-4. Effective and advantageous cooling is achieved by direct contact comprising at least partial immersion of the power transistor(s) in each of Refrigerants 1-4. The power transistor(s) are cooled with each of Refrigerants 1-4 and the power transistor(s) operate effectively, efficiently, safely and reliably while at least partially immersed in each of Refrigerants 1-4, and the power transistor(s) are kept in the most desired operating temperature range while performing its functions.

### Example 5I—Single Phase (Sensible Heat) Power Control Semiconductor Cooling Using Each of Refrigerants 1-4

Example 5B is repeated, except the cooling is applied to one or more power control semiconductor(s) using each of Refrigerants 1-4. Effective and advantageous cooling is achieved by direct contact comprising at least partial immersion of the power control semiconductor(s) in each of Refrigerants 1-4. The power control semiconductor(s) are cooled with each of Refrigerants 1-4 and the power control semiconductor(s) operate effectively, efficiently, safely and reliably while at least partially immersed in each of Refrigerants 1-4, and the power control semiconductor(s) are kept in the most desired operating temperature range while performing its functions.

### Example 5J—Single Phase (Sensible Heat) Power Transformer Cooling Using Each of Refrigerants 1-4

Example 5B is repeated, except the cooling is applied to one or more power transformer(s) using each of Refrigerants 1-4. Effective and advantageous cooling is achieved by direct contact comprising at least partial immersion of the power transformer(s) in each of Refrigerants 1-4. The power transformer(s) are cooled with each of Refrigerants 1-4 and the power transformer(s) operate effectively, efficiently, safely and reliably while at least partially immersed in each of Refrigerants 1-4, and the power transformer(s) are kept in the most desired operating temperature range while performing its functions.

### Example 5K—Single Phase (Sensible Heat) Printed Circuit Board Cooling Using Each of Refrigerants 1-4

Example 5B is repeated, except the cooling is applied to one or more printed circuit board(s) using each of Refrigerants 1-4. Effective and advantageous cooling is achieved by direct contact comprising at least partial immersion of the printed circuit board(s) in each of Refrigerants 1-4. The printed circuit board(s) are cooled with each of Refrigerants 1-4 and the printed circuit board(s) operate effectively, efficiently, safely and reliably while at least partially immersed in each of Refrigerants 1-4, and the printed circuit board(s) are kept in the most desired operating temperature range while performing its functions.

### Example 5L—Single Phase (Sensible Heat) Laser Cooling Using Each of Refrigerants 1-4

Example 5B is repeated, except the cooling is applied to one or more laser(s) using each of Refrigerants 1-4. Effective and advantageous cooling is achieved by direct contact comprising at least partial immersion of the laser(s) in each of Refrigerants 1-4. The printed laser(s) are cooled with each of Refrigerants 1-4 and the laser(s) operate effectively, efficiently, safely and reliably while at least partially immersed in each of Refrigerants 1-4, and the laser(s) are kept in the most desired operating temperature range while performing its functions.

### Example 5M—Single Phase (Sensible Heat) Multi-Chip Module Cooling Using Each of Refrigerants 1-4

Example 5B is repeated, except the cooling is applied to one or more multi-chip modules(s) using each of Refrigerants 1-4. Effective and advantageous cooling is achieved by direct contact comprising at least partial immersion of the multi-chip modules(s) in each of Refrigerants 1-4. The multi-chip modules(s) are cooled with each of Refrigerants 1-4 and the multi-chip modules(s) operate effectively, efficiently, safely and reliably while at least partially immersed in each of Refrigerants 1-4, and the multi-chip modules(s) are kept in the most desired operating temperature range while performing its functions.

### Example 5N—Single Phase (Sensible Heat) LED Cooling Using Each of Refrigerants 1-4

Example 5B is repeated, except the cooling is applied to one or more LED(s) using each of Refrigerants 1-4. Effective and advantageous cooling is achieved by direct contact comprising at least partial immersion of the LED(s) in each of Refrigerants 1-4. The LED(s) are cooled with each of Refrigerants 1-4 and the LED(s) operate effectively, efficiently, safely and reliably while at least partially immersed in each of Refrigerants 1-4, and the LED(s) are kept in the most desired operating temperature range while performing its functions.

### Example 5O—Single Phase (Sensible Heat) Electrical Distribution Switch Gear Cooling Using Each of Refrigerants 1-4

Example 5A is repeated, except the cooling is applied to one or more electrical vehicle batteries using each of Refrigerants 1-4. Effective and advantageous cooling is achieved by direct contact comprising at least partial immersion of the EV battery in each of Refrigerants 1-4. The EV Battery ais cooled with each of Refrigerants 1-4 and the EV battery operates effectively, efficiently, safely and reliably while at least partially immersed in each of Refrigerants 1-4, and the EV Battery is kept in the most desired operating temperature range while performing its functions.

### Example 5P—Single Phase (Sensible Heat) Electrical Distribution Switch Gear Cooling Using Each of Refrigerants 1-4

Example 5B is repeated, except the cooling is applied to one or more electrochemical cell(s) using each of Refrigerants 1-4. Effective and advantageous cooling is achieved by direct contact comprising at least partial immersion of the electrochemical cell(s) in each of Refrigerants 1-4. The electrochemical cell(s) are cooled with each of Refrigerants 1-4 absorbing heat via and the electrochemical cell(s) operate effectively, efficiently, safely and reliably while at least partially immersed in each of Refrigerants 1-4, and the electrochemical cell(s) are kept in the most desired operating temperature range while performing its functions.

### Example 6A—Two Phase (Latent Heat) Immersion Cooling in Battery Applications Using Each of Refrigerants 1-4

Example 3A is repeated except cooling comprising two phase (latent heat) cooling is provided to the batteries using each of Refrigerants 1-4. Effective and advantageous cooling is achieved by direct contact comprising at least partial immersion of the batteries in each of Refrigerants 1-4. The batteries are cooled with each of Refrigerants 1-4 and the batteries operate effectively, efficiently, safely and reliably while at least partially immersed in each of Refrigerants 1-4, and the batteries are kept in the most desired operating temperature range while performing its functions.

### Example 7A—Two Phase (Latent Heat) Immersion Cooling in Data Center Applications Using Each of Refrigerants 1-4

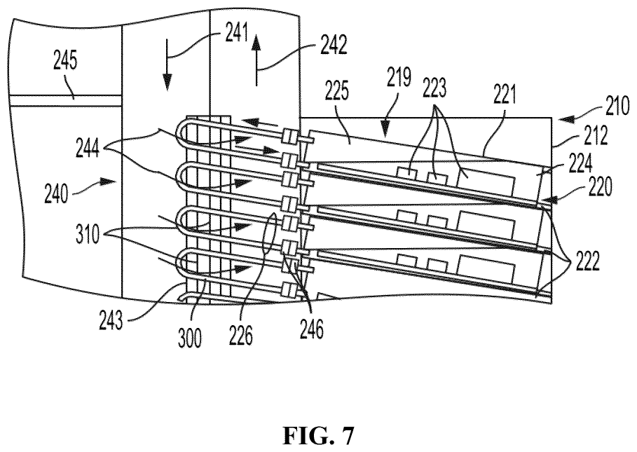

An example of data center cooling is provided, making reference to FIG. 7. A data center, generally denoted 200, includes a plurality of electronic subsystems 220 contained in one or more of electronics racks 210. At least one, and preferably a plurality, and preferably all, of the electronic subsystems 220 are associated with a cooling station 240 that includes (in one embodiment) a vertically extending, liquid-to-air heat exchanger 243 and supply and return ducting 241, 242 for directing a cooling airflow 244 across liquid-to-air heat exchanger 243. A cooling subsystem 219 is associated with at least one, and preferably a plurality, and preferably all, of the multiple electronic subsystems 220. In a preferred embodiment, as shown in FIG. 7, all of the subsystem 220 are associated with the cooling station 240 and a cooling subsystem 219. Each cooling subsystem 219 comprises (in this embodiment) a housing 221 (which preferably is a low-pressure housing) which encloses a respective electronic subsystem 220 comprising a plurality of electronic components 223. The electronic components are in operation as part of the data center and are generating heat as a result of performing their function in the data center. The components include, by way of example, printed circuit boards, microprocessor modules, and memory devices. Each electronic subsystem has, as it is operating, its heat generating components immersed in a thermal management fluid of the present invention 224, including each of Refrigerants 1-4. The fluid 224 boils in typical operation, generating dielectric vapor 225 according to the present invention. In the illustrated embodiment, electronic subsystems 220 are angled by providing upward-sloped support rails 222 within electronics rack 210 to accommodate the electronic subsystems 220 at an angle. Angling of the electronic subsystems as illustrated facilitates buoyancy-driven circulation of vapor 225 between the cooling subsystem 219 and the liquid-to-air heat exchanger 243 of the associated local cooling station 240. However, the excellent results according to the present invention and the present example are achieved equally well when such angling is not used. Multiple coolant loops 226 are coupled in fluid and thermal contact with the liquid-cooled electronic subsystems and a respective portion of liquid-to-air heat exchanger 243. In particular, multiple tubing sections 300 pass through liquid-to-air heat exchanger 243, which in this example includes a plurality of air-cooling fins 310. Vapor 225 is buoyancy-driven from housing 221 to the corresponding tubing section 300 of liquid-to-air heat exchanger 243, where the vapor condenses and is then returned as liquid to the associated liquid-cooled electronics subsystem. Cooling airflow 244 is provided in parallel to the supply ducting 241 of multiple local cooling stations 240 of data center 200, and the heated airflow is exhausted via return ducting 242. The equipment as described herein, but not the fluid of the present invention, is disclosed in US 2013/0019614, which is incorporated herein by reference.

The system as described above is operated with a thermal management fluid consisting of the present invention, including each of Refrigerants 1-4 and ambient air as the heat sink for the condenser, and this system operates effectively, efficiently, safely and reliably maintain the electronic components in the most desired operating temperature range while the system is performing its function in the operating data center.

### Example 7B—Two Phase (Latent Heat) Immersion Semiconductor Integrated Circuit Cooling Using Each of Refrigerants 1-4

Example 7A is repeated, except the cooling is applied to one or more semiconductor integrated circuit(s) using each of Refrigerants 1-4. Effective and advantageous cooling is achieved by direct contact comprising at least partial immersion of the semiconductor integrated circuit in each of Refrigerants 1-4. The semiconductor integrated circuit(s) are cooled with each of Refrigerants 1-4 and the semiconductor integrated circuit(s) operate effectively, efficiently, safely and reliably while at least partially immersed in Refrigerants 1-4, and the semiconductor integrated circuit(s) are kept in the most desired operating temperature range while performing its functions.

### Example 7C—Two Phase (Latent Heat) Immersion Microprocessor Cooling Using Each of Refrigerants 1-4

Example 7A is repeated, except the cooling is applied to one or more microprocessor(s) using each of Refrigerants 1-4. Effective and advantageous cooling is achieved by direct contact comprising at least partial immersion of the microprocessor(s) in each of Refrigerants 1-4. The microprocessor(s) are cooled with each of Refrigerants 1-4 and the component/article/device operates effectively, efficiently, safely and reliably while at least partially immersed in Refrigerants 1-4, and the microprocessor(s) are kept in the most desired operating temperature range while performing its functions.

### Example 7D—Two Phase (Latent Heat) Immersion Electrochemical Cell Cooling Using Each of Refrigerants 1-4

Example 7A is repeated, except the cooling is applied to one or more electrochemical cell(s) using each of Refrigerants 1-4. Effective and advantageous cooling is achieved by direct contact comprising at least partial immersion of the electrochemical cell(s) in each of Refrigerants 1-4. The electrochemical cell(s) are cooled with each of Refrigerants 1-4 absorbing heat via and the electrochemical cell(s) operate effectively, efficiently, safely and reliably while at least partially immersed in each of Refrigerants 1-4, and the electrochemical cell(s) are kept in the most desired operating temperature range while performing its functions.

### Example 7E—Two Phase (Latent Heat) Immersion Fuel Cell Cooling Using Each of Refrigerants 1-4

Example 7A is repeated, except the cooling is applied to one or more fuel cell(s) using each of Refrigerants 1-4. Effective and advantageous cooling is achieved by direct contact comprising at least partial immersion of the fuel cell(s) in each of Refrigerants 1-4. The fuel cell(s) are cooled with each of Refrigerants 1-4 and the fuel cell(s) operate effectively, efficiently, safely and reliably while at least partially immersed in each of Refrigerants 1-4, and the fuel cell(s) are kept in the most desired operating temperature range while performing its functions.

### Example 7F—Two Phase (Latent Heat) Immersion Resistor Cooling Using Each of Refrigerants 1-4

Example 7A is repeated, except the cooling is applied to one or more resistor(s) using each of Refrigerants 1-4. Effective and advantageous cooling is achieved by direct contact comprising at least partial immersion of the resistor(s) in each of Refrigerants 1-4. The resistor(s) are cooled with each of Refrigerants 1-4 and the resistor(s) operate effectively, efficiently, safely and reliably while at least partially immersed in each of Refrigerants 1-4, and the resistor(s) are kept in the most desired operating temperature range while performing its functions.

### Example 7G—Two Phase (Latent Heat) Immersion Power Transistor Cooling Using Each of Refrigerants 1-4

Example 7A is repeated, except the cooling is applied to one or more power transistor(s) using each of Refrigerants 1-4. Effective and advantageous cooling is achieved by direct contact comprising at least partial immersion of the power transistor(s) in each of Refrigerants 1-4. The power transistor(s) are cooled with each of Refrigerants 1-4 and the power transistor(s) operate effectively, efficiently, safely and reliably while at least partially immersed in each of Refrigerants 1-4, and the power transistor(s) are kept in the most desired operating temperature range while performing its functions.

### Example 7H—Two Phase (Latent Heat) Immersion Power Control Semiconductor Cooling Using Each of Refrigerants 1-4

Example 7A is repeated, except the cooling is applied to one or more power control semiconductor(s) using each of Refrigerants 1-4. Effective and advantageous cooling is achieved by direct contact comprising at least partial immersion of the power control semiconductor(s) in each of Refrigerants 1-4. The power control semiconductor(s) are cooled with each of Refrigerants 1-4 and the power control semiconductor(s) operate effectively, efficiently, safely and reliably while at least partially immersed in each of Refrigerants 1-4, and the power control semiconductor(s) are kept in the most desired operating temperature range while performing its functions.

### Example 7I—Two Phase (Latent Heat) Immersion Power Transformer Cooling Using Each of Refrigerants 1-4

Example 7A is repeated, except the cooling is applied to one or more power transformer(s) using each of Refrigerants 1-4. Effective and advantageous cooling is achieved by direct contact comprising at least partial immersion of the power transformer(s) in each of Refrigerants 1-4. The power transformer(s) are cooled with each of Refrigerants 1-4 and the power transformer(s) operate effectively, efficiently, safely and reliably while at least partially immersed in each of Refrigerants 1-4, and the power transformer(s) are kept in the most desired operating temperature range while performing its functions.

### Example 7J—Two Phase (Latent Heat) Immersion Printed Circuit Board Cooling Using Each of Refrigerants 1-4

Example 7A is repeated, except the cooling is applied to one or more printed circuit board(s) using each of Refrigerants 1-4. Effective and advantageous cooling is achieved by direct contact comprising at least partial immersion of the printed circuit board(s) in each of Refrigerants 1-4. The printed circuit board(s) are cooled with each of Refrigerants 1-4 and the printed circuit board(s) operate effectively, efficiently, safely and reliably while at least partially immersed in each of Refrigerants 1-4, and the printed circuit board(s) are kept in the most desired operating temperature range while performing its functions.

### Example 7K—Two Phase (Latent Heat) Immersion Laser Cooling Using Each of Refrigerants 1-4

Example 7A is repeated, except the cooling is applied to one or more laser(s) using each of Refrigerants 1-4. Effective and advantageous cooling is achieved by direct contact comprising at least partial immersion of the laser(s) in each of Refrigerants 1-4. The printed laser(s) are cooled with each of Refrigerants 1-4 and the laser(s) operate effectively, efficiently, safely and reliably while at least partially immersed in each of Refrigerants 1-4, and the laser(s) are kept in the most desired operating temperature range while performing its functions.

### Example 7L—Two Phase (Latent Heat) Immersion Multi-chip Module Cooling Using Each of Refrigerants 1-4

Example 7A is repeated, except the cooling is applied to one or more multi-chip modules(s) using each of Refrigerants 1-4. Effective and advantageous cooling is achieved by direct contact comprising at least partial immersion of the multi-chip modules(s) in each of Refrigerants 1-4. The multi-chip modules(s) are cooled with each of Refrigerants 1-4 and the multi-chip modules(s) operate effectively, efficiently, safely and reliably while at least partially immersed in each of Refrigerants 1-4, and the multi-chip modules(s) are kept in the most desired operating temperature range while performing its functions.

### Example 7M—Two Phase (Latent Heat) Immersion LED Cooling Using Each of Refrigerants 1-4

Example 7A is repeated, except the cooling is applied to one or more LED(s) using each of Refrigerants 1-4. Effective and advantageous cooling is achieved by direct contact comprising at least partial immersion of the LED(s) in each of Refrigerants 1-4. The LED(s) are cooled with each of Refrigerants 1-4 and the LED(s) operate effectively, efficiently, safely and reliably while at least partially immersed in each of Refrigerants 1-4, and the LED(s) are kept in the most desired operating temperature range while performing its functions.

### Example 8—Method and Use of Each of Refrigerants 1-4 in Fabrication of an Electronic Component

An electronic component which undergoes processing that includes thermal control of the component (such as for example, in the etching of a silicon wafer as illustrated for example in FIG. 11) is contacted, directly or indirectly, by each of Refrigerants 1-5 to transfer heat between (to and/or from said electronic component) as part of the manufacturing process. Effective temperature control is provided.

### Example 9: Heat Transfer Compositions A-D Used in Orgic Rankine Cycles (ORC)

Each of Heat Transfer Compositions A-D are used as the working fluid in an Organic Rankine Cycle. Each of Refrigerants 1-4 and Heat Transfer Compositions A-D each sufficiently recovered waste heat in an Organic Rankine cycle.

### Example 10: Heat Transfer Compositions A-D Used in High Temperature Heat Pumps

Each of Heat Transfer Compositions A-D are used as the working fluid in a high temperature heat pump system. Each of Heat Transfer Compositions A-D each sufficiently provided appropriate heating the system.

### Example 11: Heat Transfer Compositions A-D Used in Secondary Loop Systems

A secondary loop system is designed, and Heat transfer compositions A-D are circulated in the secondary loop. Each of Heat Transfer Composition A-D provide sufficient heat transfer to operate in the secondary loop system.

### Example 12: Toxicity Testing

A fluid including the trans isomer of the SecBuFO refrigerant was subjected experimentally a toxicological screening study to assess in vivo acute oral toxicity at a dosage of 2,000 mg/kg/day and 4-hour acute inhalation toxicity at a concentration of 18,375 ppm. The trans SecBuFO compound passed the toxicology screening study, demonstrating that the compound as acceptable toxicity.

### Aspects

Aspect 1 is the molecule trans-1,3,4,4,5,5,5-heptafluoro-3-(trifluoromethyl)pent-1-ene, having the structure according to the formula below:

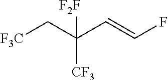

Aspect 2 is a composition comprising trans-1,3,4,4,5,5,5-heptafluoro-3-(trifluoromethyl)pent-1-ene according to Aspect 1, and at least one impurity, wherein the trans-1,3,4,4,5,5,5-heptafluoro-3-(trifluoromethyl)pent-1-ene is present in an amount greater than or equal to 98.5 wt. %, as based upon the total weight of the trans-1,3,4,4,5,5,5-heptafluoro-3-(trifluoromethyl)pent-1-ene and the one or more impurities.

Aspect 3 is the composition of Aspect 2, wherein the trans-1,3,4,4,5,5,5-heptafluoro-3-(trifluoromethyl)pent-1-ene is present in an amount greater than or equal to 99.50 wt. %, as based upon the total weight of the trans-1,3,4,4,5,5,5-heptafluoro-3-(trifluoromethyl)pent-1-ene and the one or more impurities.

Aspect 4 is the composition of Aspect 2, wherein the trans-1,3,4,4,5,5,5-heptafluoro-3-(trifluoromethyl)pent-1-ene is present in an amount greater than or equal to 99.90 wt. %, as based upon the total weight of the trans-1,3,4,4,5,5,5-heptafluoro-3-(trifluoromethyl)pent-1-ene and the one or more impurities.

Aspect 5 is the composition of Aspect 2, wherein the trans-1,3,4,4,5,5,5-heptafluoro-3-(trifluoromethyl)pent-1-ene is present in an amount greater than or equal to 99.95 wt. %, as based upon the total weight of the trans-1,3,4,4,5,5,5-heptafluoro-3-(trifluoromethyl)pent-1-ene and the one or more impurities.

Aspect 6 is the composition of any of Aspects 2-5, wherein the one or more impurities comprise at least one of cis-1,3,4,4,5,5,5-heptafluoro-3-(trifluoromethyl)pent-1-ene, partially fluorinated benzene compounds, and perfluorinated alkyl compounds.

Aspect 7: is the molecule cis-1,3,4,4,5,5,5-heptafluoro-3-(trifluoromethyl)pent-1-ene, having the structure according to the formula below:

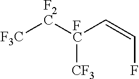

Aspect 8 is a composition comprising cis-1,3,4,4,5,5,5-heptafluoro-3-(trifluoromethyl)pent-1-ene according to Aspect 7, and at least one impurity, wherein the cis-1,3,4,4,5,5,5-heptafluoro-3-(trifluoromethyl)pent-1-ene is present in an amount greater than or equal to 98.5 wt. %, as based upon the total weight of the cis-1,3,4,4,5,5,5-heptafluoro-3-(trifluoromethyl)pent-1-ene and the one or more impurities.

Aspect 9 is the composition of Aspect 8, wherein the cis-1,3,4,4,5,5,5-heptafluoro-3-(trifluoromethyl)pent-1-ene is present in an amount greater than or equal to 99.50 wt. %, as based upon the total weight of the cis-1,3,4,4,5,5,5-heptafluoro-3-(trifluoromethyl)pent-1-ene and the one or more impurities.

Aspect 10 is the composition of Aspect 8, wherein the cis-1,3,4,4,5,5,5-heptafluoro-3-(trifluoromethyl)pent-1-ene is present in an amount greater than or equal to 99.90 wt. %, as based upon the total weight of the cis-1,3,4,4,5,5,5-heptafluoro-3-(trifluoromethyl)pent-1-ene and the one or more impurities.

Aspect 11 is the composition of Aspect 8, wherein the cis-1,3,4,4,5,5,5-heptafluoro-3-(trifluoromethyl)pent-1-ene is present in an amount greater than or equal to 99.95 wt. %, as based upon the total weight of the cis-1,3,4,4,5,5,5-heptafluoro-3-(trifluoromethyl)pent-1-ene and the one or more impurities.

Aspect 12: is the composition of any of Aspects 8-11, wherein the one or more impurities comprise at least trans-1,3,4,4,5,5,5-heptafluoro-3-(trifluoromethyl)pent-1-ene.

Aspect 13 is a method for synthesizing the molecule of Aspect 1 and/or Aspect 7, comprising: reacting 2-iodononafluorobutane with vinylidene fluoride to yield 1,1,1,2,2,3,5-heptafluoro-5-iodo-3-(trifluoromethyl) pentane; reacting 1,1,1,2,2,3,5-heptafluoro-5-iodo-3-(trifluoromethyl) pentane with a base in the presence of a catalyst; and collecting trans-1,3,4,4,5,5,5-heptafluoro-3-(trifluoromethyl)pent-1-ene and/or cis-1,3,4,4,5,5,5-heptafluoro-3-(trifluoromethyl)pent-1-ene.

Aspect 14 is the method of Aspect 13, further comprising at least one of the following: the base comprises potassium hydroxide (KOH); the catalyst comprises tetrabutylammonium chloride (TBAC); the second reacting step is carried out at a temperature of from 50° C. to 100° C.; and the trans-1,3,4,4,5,5,5-heptafluoro-3-(trifluoromethyl)pent-1-ene and/or cis-1,3,4,4,5,5,5-heptafluoro-3-(trifluoromethyl)pent-1-ene is collected in the gas phase and further condensed to a liquid.

Aspect 15: The molecule trans-1,4,4,4-tetrafluoro-2-2-bis(trifluoromethyl)butane, having the structure according to the formula below:

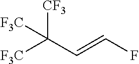 [SMILES: F/C=C/C(C(F)(F)F)(C(F)(F)F)C(F)(F)F]

Aspect 16 is a composition comprising trans-1,4,4,4-tetrafluoro-2-2-bis(trifluoromethyl)butane according to Aspect 15, and at least one impurity, wherein the trans-1,4,4,4-tetrafluoro-2-2-bis(trifluoromethyl)butane is present in an amount greater than or equal to 98.50 wt. %, as based upon the total weight of the trans-1,4,4,4-tetrafluoro-2-2-bis(trifluoromethyl)butane and the one or more impurities.

Aspect 17: is the composition of Aspect 16, wherein the trans-1,4,4,4-tetrafluoro-2-2-bis(trifluoromethyl)butane is present in an amount greater than or equal to 99.50 wt. %, as based upon the total weight of the trans-1,4,4,4-tetrafluoro-2-2-bis(trifluoromethyl)butane and the one or more impurities.

Aspect 18 is the composition of Aspect 16, wherein the trans-1,4,4,4-tetrafluoro-2-2-bis(trifluoromethyl)butane is present in an amount greater than or equal to 99.90 wt. %, as based upon the total weight of the trans-1,4,4,4-tetrafluoro-2-2-bis(trifluoromethyl)butane and the one or more impurities.

Aspect 19 is the composition of Aspect 16, wherein the trans-1,4,4,4-tetrafluoro-2-2-bis(trifluoromethyl)butane is present in an amount greater than or equal to 99.95 wt. %, as based upon the total weight of the trans-1,4,4,4-tetrafluoro-2-2-bis(trifluoromethyl)butane and the one or more impurities.

Aspect 20 is the composition of any of Aspects 16-19, wherein the one or more impurities comprise at least cis-1,4,4,4-tetrafluoro-2-2-bis(trifluoromethyl)butane.

Aspect 21 is the molecule cis-1,4,4,4-tetrafluoro-2-2-bis(trifluoromethyl)butane, having the structure according to the formula below:

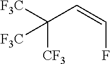 [SMILES: F/C=C\C(C(F)(F)F)(C(F)(F)F)C(F)(F)F]

Aspect 22 is a composition comprising cis-1,4,4,4-tetrafluoro-2-2-bis(trifluoromethyl)butane according to Aspect 21, and at least one impurity, wherein the cis-1,4,4,4-tetrafluoro-2-2-bis(trifluoromethyl)butane is present in an amount greater than or equal to 98.50 wt. %, as based upon the total weight of the cis-1,4,4,4-tetrafluoro-2-2-bis(trifluoromethyl)butane and the one or more impurities.

Aspect 23 is the composition of Aspect 22, wherein the cis-1,4,4,4-tetrafluoro-2-2-bis(trifluoromethyl)butane is present in an amount greater than or equal to 99.50 wt. %, as based upon the total weight of the cis-1,4,4,4-tetrafluoro-2-2-bis(trifluoromethyl)butane and the one or more impurities.

Aspect 24 is the composition of Aspect 22, wherein the cis-1,4,4,4-tetrafluoro-2-2-bis(trifluoromethyl)butane is present in an amount greater than or equal to 99.90 wt. %, as based upon the total weight of the cis-1,4,4,4-tetrafluoro-2-2-bis(trifluoromethyl)butane and the one or more impurities.

Aspect 25 is the composition of Aspect 22, wherein the cis-1,4,4,4-tetrafluoro-2-2-bis(trifluoromethyl)butane is present in an amount greater than or equal to 99.95 wt. %, as based upon the total weight of the cis-1,4,4,4-tetrafluoro-2-2-bis(trifluoromethyl)butane and the one or more impurities.

Aspect 26 is the composition of any of Aspects 22-25, wherein the one or more impurities comprise at least cis-1,4,4,4-tetrafluoro-2-2-bis(trifluoromethyl)butane.

Aspect 27: is a method for synthesizing the molecule of Aspect 15 and/or Aspect 21, comprising: reacting hexafluoroacetone with carbon tetrabromide (CBr4) and triphenyl phosphine to yield 1,1-dibromo-3,3,3-trifluoro-2-(trifluoromethyl)prop-1-ene; reacting 1,1-dibromo-3,3,3-trifluoro-2-(trifluoromethyl)prop-1-ene with cesium fluoride to yield 4-halo-1,1,1,4-tetrafluoro-2,2-bis(trifluoromethyl)butane; reacting 4-halo-1,1,1,4-tetrafluoro-2,2-bis(trifluoromethyl)butane with a base in the presence of a catalyst; and collecting trans-1,4,4,4-tetrafluoro-3,3-bis(trifluoromethyl)but-1-ene and/or cis-1,4,4,4-tetrafluoro-3,3-bis(trifluoromethyl)but-1-ene.

Aspect 28 is the method of Aspect 27, further comprising at least one of the following: the base is potassium hydroxide (KOH); the catalyst is tetrabutylammonium chloride (TBAC); the reacting step is carried out at a temperature of from 50° C. to 100° C.; and the trans-1,4,4,4-tetrafluoro-3,3-bis(trifluoromethyl)but-1-ene and/or cis-1,4,4,4-tetrafluoro-3,3-bis(trifluoromethyl)but-1-ene is collected in the gas phase and further condensed to a liquid.

Aspect 29: is a method of heating and/or cooling of electronic components, articles and/or devices during the manufacture and/or operation thereof comprising: providing an electronic component, article or device which is being manufactured and/or being operated for its intended purpose; and transferring heat to and/or from said electronic component, article and/or device during at least a portion of said manufacturing and/or operating process by directly or indirectly transferring heat between said electronic component, article and/or device and a refrigerant fluid comprising the molecule of any of Aspects 1, 7, 15, or 21 and combinations thereof.

Aspect 30 is the method of Aspect 29, wherein said step of transferring heat comprises direct heat transfer.

Aspect 31 is the method of Aspect 29, wherein said step of transferring heat comprises indirect heat transfer.

Aspect 32 is the method of any of aspects 29-31, wherein said refrigerant fluid comprises from about 0.01 percent by weight to less than about 30 percent by weight of the molecule of any of claims 1, 7, 15, or 21 and combinations thereof, as based upon to the total weight of the refrigerant composition.

Aspect 33 is the method of any of claims 29-32, wherein said refrigerant fluid comprises from about 30% by weight to less than about 70 percent by weight of the molecule of any of claims 1, 7, 15, or 21 and combinations thereof, as based upon to the total weight of the refrigerant composition.

Aspect 34 is the method of any of claims 29-33, wherein said refrigerant fluid comprises from about 70 percent by weight to less than 100 percent by weight of the molecule of any of claims 1, 7, 15, or 21 and combinations thereof, as based upon to the total weight of the refrigerant composition.

Aspect 35 is the method of any of Aspects 29-34, wherein said refrigerant fluid consists essentially of the molecule of any of Aspects 1, 7, 15, or 21 and combinations thereof.

Aspect 36 is the method of any of Aspects 29-35, wherein said refrigerant fluid consists of the molecule of any of Aspects 1, 7, 15, or 21 and combinations thereof.

Aspect 37 is the method of any of Aspects 29-36, wherein said step of transferring heat comprises cooling said electronic component, article and/or device by transferring heat from said electronic component, article and/or device to said refrigerant during at least a portion of said manufacturing and/or operating process.

Aspect 38 is the method of any of Aspects 29-37, wherein said providing step comprises providing an electronic component comprising an integrated circuit.

Aspect 39 is the method of any of Aspects 29-38, wherein said electronic component is in a server.

Aspect 40 is the method of any of Aspects 29-39, wherein the server is a data center server.

Aspect 41 is the method of any of Aspects 29-40, wherein said providing step comprises providing a battery.

Aspect 42 is the method of any of Aspects 29-41, wherein said battery is in an electric or hybrid vehicle.

Aspect 43 is the method of any of Aspects 29-42, wherein said step of transferring heat comprises at least partially immersing said electronic component, article and/or device in said refrigerant fluid.

Aspect 44 is the method of any of Aspects 29-43, wherein said step of transferring heat comprises fully immersing said electronic component, article and/or device in said refrigerant fluid.

Aspect 45: is the method of any of Aspects 29-44, wherein said providing step comprises providing an electronic article during the process of manufacturing said electronic article.

Aspect 46 is the method of Aspect 45, wherein said electronic article is a wafer.

Aspect 47 is the method of Aspect 46, wherein said process of manufacturing said wafer comprises etching a portion of said wafer.

Aspect 48: A heat transfer composition comprising a refrigerant and at least one lubricant, wherein the refrigerant comprises the molecule of any of Aspects 1, 7, 15, or 21, and combinations thereof.

Aspect 49 is the heat transfer composition of Aspect 48, wherein the one or more lubricants comprise a polyalphaolefin (PAO), a polyol ester (POE), Polyvinyl Ether (PVE) and/or a mineral oil.

Aspect 50 is a method of operating a high temperature heat pump including a fluid circuit including a compressor, condenser, expansion device and evaporator, comprising: circulating the heat transfer composition of Aspect 48 or 49 through the fluid circuit.

Aspect 51 is a method for converting thermal energy to mechanical energy in an organic Rankine cycle system including a fluid circuit including a pump, evaporator, expansion device and condenser, comprising circulating the heat transfer composition of Aspects 48 or 49 through the fluid circuit.

Aspect 52 is a heat transfer composition comprising the molecule and/or composition of any one of Aspects 1-28

Aspect 53 is a method of heating and/or cooling of electronic components, articles and/or devices during the manufacture and/or operation thereof comprising the molecule and/or composition of any one of aspects any one of Aspects 1-28.

Aspect 54 is the molecule trans-1,3,4,4,5,5,5-heptafluoro-3-(trifluoromethyl)pent-1-ene, having the structure according to the formula below:

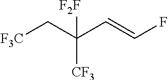

Aspect 55 is the molecule of Aspect 54, wherein the molecule is included in a composition comprising the molecule and at least one impurity, wherein the molecule is present in an amount greater than or equal to 99.85 wt. %, as based upon the total weight of the molecule and the one or more impurities.

Aspect 56 is the molecule of Aspect 54, wherein the molecule is included in a composition comprising the molecule and at least one impurity, wherein the molecule is present in an amount greater than or equal to 99.90 wt. %, as based upon the total weight of the molecule and the one or more impurities.

Aspect 57 is the molecule of Aspect 54, wherein the molecule is included in a composition comprising the molecule and at least one impurity, wherein the molecule is present in an amount greater than or equal to 99.95 wt. %, as based upon the total weight of the molecule and the one or more impurities.

Aspect 58 is the molecule of Aspect 54, wherein the molecule is included in a composition comprising the molecule and one or more impurities, the one or more impurities comprising at least one of cis-1,3,4,4,5,5,5-heptafluoro-3-(trifluoromethyl)pent-1-ene, partially fluorinated benzene compounds, and perfluorinated alkyl compounds.

Aspect 59 is a method of heating and/or cooling of electronic components, articles and/or devices during the manufacture and/or operation thereof comprising: providing an electronic component, article or device which is being manufactured and/or being operated for its intended purpose; and transferring heat to and/or from said electronic component, article and/or device during at least a portion of said manufacturing and/or operating process by directly or indirectly transferring heat between said electronic component, article and/or device and a refrigerant fluid comprising at least one refrigerant molecule selected from:
trans-1,3,4,4,5,5,5-heptafluoro-3-(trifluoromethyl)pent-1-ene, having the structure according to the formula below:

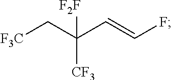

cis-1,3,4,4,5,5,5-heptafluoro-3-(trifluoromethyl)pent-1-ene having the structure according to the formula below:

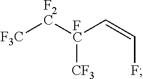

trans-1,4,4,4-tetrafluoro-2-2-bis(trifluoromethyl)butane having the structure according to the formula below:

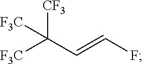 [SMILES: F/C=C/C(C(F)(F)F)(C(F)(F)F)C(F)(F)F]

cis-1,4,4,4-tetrafluoro-2-2-bis(trifluoromethyl)butane having the structure according to the formula below:

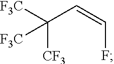 [SMILES: F/C=C\C(C(F)(F)F)(C(F)(F)F)C(F)(F)F]

and combinations of the foregoing.

Aspect 60 is the method of Aspect 59, wherein said refrigerant fluid comprises from about 0.01 percent by weight to less than about 30 percent by weight of the at least one refrigerant molecule, as based on the total weight of the refrigerant fluid.

Aspect 61 is the method of Aspect 59, wherein the refrigerant fluid comprises from about 30 percent by weight to less than about 70 percent by weight of the at least one refrigerant molecule, as based on the total weight of the refrigerant fluid.

Aspect 62 is the method of Aspect 59, wherein said refrigerant fluid comprises from about 70 percent by weight to less than 100 percent by weight of the at least one refrigerant molecule, based on the total weight of the refrigerant fluid.

Aspect 63 is the method of any one of aspects Aspect 59-62, wherein said refrigerant fluid consists essentially of the at least one refrigerant molecule.

Aspect 64 is the method of any one of aspects Aspect 59-63, wherein said refrigerant fluid consists of the at least one refrigerant molecules.

Aspect 65 is a heat transfer composition comprising a refrigerant and at least one lubricant, wherein the refrigerant comprises at least one refrigerant molecule selectee from:
trans-1,3,4,4,5,5,5-heptafluoro-3-(trifluoromethyl)pent-1-ene, having the structure according to the formula below:

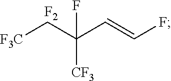

cis-1,3,4,4,5,5,5-heptafluoro-3-(trifluoromethyl)pent-1-ene having the structure according to the formula below:

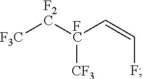

trans-1,4,4,4-tetrafluoro-2-2-bis(trifluoromethyl)butane having the structure according to the formula below:

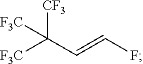 [SMILES: F/C=C/C(C(F)(F)F)(C(F)(F)F)C(F)(F)F]

cis-1,4,4,4-tetrafluoro-2-2-bis(trifluoromethyl)butane having the structure according to the formula below:

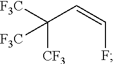 [SMILES: F/C=C\C(C(F)(F)F)(C(F)(F)F)C(F)(F)F]

and combinations of the foregoing.

Aspect 66 is the heat transfer composition of Aspect 65, wherein the one or more lubricants comprise a polyalphaolefin (PAO), a polyol ester (POE), Polyvinyl Ether (PVE) and/or a mineral oil.

LENGTHY TABLES
The patent application contains a lengthy table section. A copy of the table is available in electronic form from the USPTO web site (https://seqdata.uspto.gov/?pageRequest=docDetail&DocID=US20250297152A1). An electronic copy of the table will also be available from the USPTO upon request and payment of the fee set forth in 37 CFR 1.19(b)(3).

1. The molecule trans-1,3,4,4,5,5,5-heptafluoro-3-(trifluoromethyl)pent-1-ene, having the structure according to the formula below:

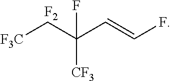

2. The molecule of claim 1, wherein the molecule is included in a composition comprising the molecule and at least one impurity, wherein the molecule is present in an amount greater than or equal to 99.85 wt. %, based on the total weight of the molecule and the at least one impurity.

3. The molecule of claim 1, wherein the molecule is included in a composition comprising the molecule and at least one impurity, wherein the molecule is present in an amount greater than or equal to 99.90 wt. %, as based upon the total weight of the molecule and the at least one impurities.

4. The molecule of claim 1, wherein the molecule is included in a composition comprising the molecule and at least one impurity, wherein the molecule is present in an amount greater than or equal to 99.95 wt. %, as based upon the total weight of the molecule and the at least one impurities.

5. The molecule of claim 1, wherein the molecule is included in a composition comprising the molecule and one or more impurities, the one or more impurities comprising at least one of cis-1,3,4,4,5,5,5-heptafluoro-3-(trifluoromethyl)pent-1-ene, partially fluorinated benzene compounds, and perfluorinated alkyl compounds.

6. A method of heating and/or cooling of electronic components, articles and/or devices during the manufacture and/or operation thereof comprising:
providing an electronic component, article or device which is being manufactured and/or being operated for its intended purpose; and
transferring heat to and/or from said electronic component, article and/or device during at least a portion of said manufacturing and/or operating process by directly or indirectly transferring heat between said electronic component, article and/or device and a refrigerant fluid comprising at least one refrigerant molecule selected from:
trans-1,3,4,4,5,5,5-heptafluoro-3-(trifluoromethyl)pent-1-ene, having the structure according to the formula below: 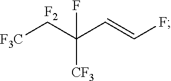
cis-1,3,4,4,5,5,5-heptafluoro-3-(trifluoromethyl)pent-1-ene having the structure according to the formula below: 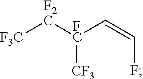
trans-1,4,4,4-tetrafluoro-2-2-bis(trifluoromethyl)butane having the structure according to the formula below: 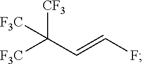 [SMILES: F/C=C/C(C(F)(F)F)(C(F)(F)F)C(F)(F)F]
cis-1,4,4,4-tetrafluoro-2-2-bis(trifluoromethyl)butane having the structure according to the formula below: 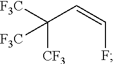 [SMILES: F/C=C\C(C(F)(F)F)(C(F)(F)F)C(F)(F)F]
and
combinations of the foregoing.

7. The method of claim 6, wherein said refrigerant fluid comprises from about 0.01 percent by weight to less than about 30 percent by weight of the at least one refrigerant molecule, based on the total weight of the refrigerant fluid.

8. The method of claim 6, wherein the refrigerant fluid comprises from about 30 percent by weight to less than about 70 percent by weight of the at least one refrigerant molecule, based on the total weight of the refrigerant fluid.

9. The method of claim 6, wherein said refrigerant fluid comprises from about 70 percent by weight to less than 100 percent by weight of the at least one refrigerant molecule, based on to the total weight of the refrigerant fluid.

10. The method of claim 6, wherein said refrigerant fluid consists essentially of the at least one refrigerant molecule.

11. The method of claim 6, wherein said refrigerant fluid consists of the at least one refrigerant molecule.

12. A heat transfer composition comprising a refrigerant and at least one lubricant, wherein the refrigerant comprises at least one refrigerant molecule selected from:
trans-1,3,4,4,5,5,5-heptafluoro-3-(trifluoromethyl)pent-1-ene, having the structure according to the formula below: 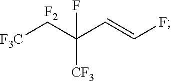
cis-1,3,4,4,5,5,5-heptafluoro-3-(trifluoromethyl)pent-1-ene having the structure according to the formula below: 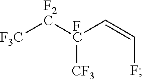
trans-1,4,4,4-tetrafluoro-2-2-bis(trifluoromethyl)butane having the structure according to the formula below: 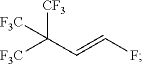 [SMILES: F/C=C/C(C(F)(F)F)(C(F)(F)F)C(F)(F)F]
cis-1,4,4,4-tetrafluoro-2-2-bis(trifluoromethyl)butane having the structure according to the formula below: 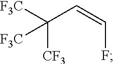 [SMILES: F/C=C\C(C(F)(F)F)(C(F)(F)F)C(F)(F)F]
and
combinations of the foregoing.

13. The heat transfer composition of claim 12, wherein the one or more lubricants comprise a polyalphaolefin (PAO), a polyol ester (POE), Polyvinyl Ether (PVE) and/or a mineral oil.
